SEQUENCE LISTING (378 sequences)

SEQ ID NO: 1 - protein, 109 aa, synthetic construct
  REGION 1..109: note = Synthetic Construct
  VARIANT 14: note = MISC_FEATURE - Xaa is Phe or Tyr
  VARIANT 15: note = MISC_FEATURE - Xaa is Phe or Tyr
  VARIANT 20: note = MISC_FEATURE - Xaa is Glu or Ala
  VARIANT 21: note = MISC_FEATURE - Xaa is Lys or Leu
  VARIANT 22: note = MISC_FEATURE - Xaa is Asp or Glu
  VARIANT 23: note = MISC_FEATURE - Xaa is Arg or Ala
  VARIANT 31: note = MISC_FEATURE - Xaa is Pro or Arg
  VARIANT 33: note = MISC_FEATURE - Xaa is Tyr or Glu
  VARIANT 35: note = MISC_FEATURE - Xaa is Asp or Glu
  VARIANT 36: note = MISC_FEATURE - Xaa is Lys or Gln
  VARIANT 37: note = MISC_FEATURE - Xaa is Asp or Ala
  VARIANT 38: note = MISC_FEATURE - Xaa is Lys or Ala
  VARIANT 39: note = MISC_FEATURE - Xaa is Arg or Ala
  VARIANT 40: note = MISC_FEATURE - Xaa is Arg or Leu
  VARIANT 43: note = MISC_FEATURE - Xaa is Phe or Tyr
  VARIANT 47: note = MISC_FEATURE - Xaa is Lys, Arg, or Ala
  VARIANT 57: note = MISC_FEATURE - Xaa is Lys, Ala, Tyr, Phe, or Ile
  VARIANT 58: note = MISC_FEATURE - Xaa is Gln or Lys
  VARIANT 61: note = MISC_FEATURE - Xaa is Trp or Ala
  VARIANT 62: note = MISC_FEATURE - Xaa is Leu or Ala
  VARIANT 63: note = MISC_FEATURE - Xaa is Asp, Lys, Arg, Ala, Phe, Gly, Met, Asn, or Ile
  VARIANT 65: note = MISC_FEATURE - Xaa is Ile, Phe, or Ala
  VARIANT 76: note = MISC_FEATURE - Xaa is Lys or Thr
  VARIANT 77: note = MISC_FEATURE - Xaa is Lys or Glu
  VARIANT 78: note = MISC_FEATURE - Xaa is Asp or Glu
  VARIANT 79: note = MISC_FEATURE - Xaa is Ser or Asn
  VARIANT 81: note = MISC_FEATURE - Xaa is Glu or Gln
GAILGRSETQECLXXNANWXXXXTNQTGVEXCXGXXXXXXHCXATWXNISGSIEIVXXGCXXXDXNCYDRTDCVEXXXXPXVYFCCCEGNMCNEKFSYFPEMEVTQPTS

SEQ ID NO: 2 - protein, 109 aa, synthetic construct
  REGION 1..109: note = Synthetic Construct
  VARIANT 15: note = MISC_FEATURE - Xaa is Phe or Tyr
  VARIANT 20: note = MISC_FEATURE - Xaa is Glu or Ala
  VARIANT 21: note = MISC_FEATURE - Xaa is Lys or Leu
  VARIANT 22: note = MISC_FEATURE - Xaa is Asp or Glu
  VARIANT 23: note = MISC_FEATURE - Xaa is Arg or Ala
  VARIANT 31: note = MISC_FEATURE - Xaa is Pro or Arg
  VARIANT 33: note = MISC_FEATURE - Xaa is Tyr or Glu
  VARIANT 35: note = MISC_FEATURE - Xaa is Asp or Glu
  VARIANT 37: note = MISC_FEATURE - Xaa is Asp or Ala
  VARIANT 38: note = MISC_FEATURE - Xaa is Lys or Ala
  VARIANT 39: note = MISC_FEATURE - Xaa is Arg or Ala
  VARIANT 40: note = MISC_FEATURE - Xaa is Arg or Leu
  VARIANT 43: note = MISC_FEATURE - Xaa is Phe or Tyr
  VARIANT 47: note = MISC_FEATURE - Xaa is Lys, Arg, or Ala
  VARIANT 57: note = MISC_FEATURE - Xaa is Lys, Ala, Tyr, Phe, or Ile
  VARIANT 58: note = MISC_FEATURE - Xaa is Gln or Lys
  VARIANT 61: note = MISC_FEATURE - Xaa is Trp or Ala
  VARIANT 62: note = MISC_FEATURE - Xaa is Leu or Ala
  VARIANT 63: note = MISC_FEATURE - Xaa is Asp, Lys, Arg, Ala, Phe, Gly, Met, Asn, or Ile
  VARIANT 65: note = MISC_FEATURE - Xaa is Ile, Phe, or Ala
  VARIANT 76: note = MISC_FEATURE - Xaa is Lys or Thr
  VARIANT 77: note = MISC_FEATURE - Xaa is Lys or Glu
  VARIANT 78: note = MISC_FEATURE - Xaa is Asp or Glu
  VARIANT 79: note = MISC_FEATURE - Xaa is Ser or Asn
  VARIANT 81: note = MISC_FEATURE - Xaa is Glu or Gln
GAILGRSETQECLFXNANWXXXXTNQTGVEXCXGXKXXXXHCXATWXNISGSIEIVXXGCXXXDXNCYDRTDCVEXXXXPXVYFCCCEGNMCNEKFSYFPEMEVTQPTS

SEQ ID NO: 3 - protein, 109 aa, synthetic construct
  REGION 1..109: note = Synthetic Construct
  VARIANT 15: note = MISC_FEATURE - Xaa is Phe or Tyr
  VARIANT 21: note = MISC_FEATURE - Xaa is Lys or Leu
  VARIANT 22: note = MISC_FEATURE - Xaa is Asp or Glu
  VARIANT 31: note = MISC_FEATURE - Xaa is Pro or Arg
  VARIANT 33: note = MISC_FEATURE - Xaa is Tyr or Glu
  VARIANT 35: note = MISC_FEATURE - Xaa is Asp or Glu
  VARIANT 40: note = MISC_FEATURE - Xaa is Arg or Leu
  VARIANT 43: note = MISC_FEATURE - Xaa is Phe or Tyr
  VARIANT 47: note = MISC_FEATURE - Xaa is Lys, Arg, or Ala
  VARIANT 58: note = MISC_FEATURE - Xaa is Gln or Lys
  VARIANT 65: note = MISC_FEATURE - Xaa is Ile, Phe, or Ala
  VARIANT 76: note = MISC_FEATURE - Xaa is Lys or Thr
  VARIANT 77: note = MISC_FEATURE - Xaa is Lys or Glu
  VARIANT 78: note = MISC_FEATURE - Xaa is Asp or Glu
  VARIANT 79: note = MISC_FEATURE - Xaa is Ser or Asn
  VARIANT 81: note = MISC_FEATURE - Xaa is Glu or Gln
GAILGRSETQECLFXNANWEXXRTNQTGVEXCXGXKDKRXHCXATWXNISGSIEIVKXGCWLDDXNCYDRTDCVEXXXXPXVYFCCCEGNMCNEKFSYFPEMEVTQPTS

SEQ ID NO: 4 - protein, 109 aa, synthetic construct
  REGION 1..109: note = Synthetic Construct
  VARIANT 15: note = MISC_FEATURE - Xaa is Phe or Tyr
  VARIANT 21: note = MISC_FEATURE - Xaa is Lys or Leu
  VARIANT 31: note = MISC_FEATURE - Xaa is Pro or Arg
  VARIANT 33: note = MISC_FEATURE - Xaa is Tyr or Glu
  VARIANT 35: note = MISC_FEATURE - Xaa is Asp or Glu
  VARIANT 40: note = MISC_FEATURE - Xaa is Arg or Leu
  VARIANT 43: note = MISC_FEATURE - Xaa is Phe or Tyr
  VARIANT 47: note = MISC_FEATURE - Xaa is Lys, Arg, or Ala
  VARIANT 58: note = MISC_FEATURE - Xaa is Gln or Lys
  VARIANT 65: note = MISC_FEATURE - Xaa is Ile, Phe, or Ala
  VARIANT 76: note = MISC_FEATURE - Xaa is Lys or Thr
  VARIANT 78: note = MISC_FEATURE - Xaa is Asp or Glu
  VARIANT 79: note = MISC_FEATURE - Xaa is Ser or Asn
  VARIANT 81: note = MISC_FEATURE - Xaa is Glu or Gln
GAILGRSETQECLFXNANWEXDRTNQTGVEXCXGXKDKRXHCXATWXNISGSIEIVKXGCWLDDXNCYDRTDCVEXKXXPXVYFCCCEGNMCNEKFSYFPEMEVTQPTS

SEQ ID NO: 5 - protein, 109 aa, synthetic construct
  REGION 1..109: note = Synthetic Construct
  VARIANT 15: note = MISC_FEATURE - Xaa is Phe or Tyr
  VARIANT 21: note = MISC_FEATURE - Xaa is Lys or Leu
  VARIANT 33: note = MISC_FEATURE - Xaa is Tyr or Glu
  VARIANT 35: note = MISC_FEATURE - Xaa is Asp or Glu
  VARIANT 40: note = MISC_FEATURE - Xaa is Arg or Leu
  VARIANT 58: note = MISC_FEATURE - Xaa is Gln or Lys
  VARIANT 76: note = MISC_FEATURE - Xaa is Lys or Thr
  VARIANT 78: note = MISC_FEATURE - Xaa is Asp or Glu
  VARIANT 79: note = MISC_FEATURE - Xaa is Ser or Asn
  VARIANT 81: note = MISC_FEATURE - Xaa is Glu or Gln
GAILGRSETQECLFXNANWEXDRTNQTGVEPCXGXKDKRXHCFATWKNISGSIEIVKXGCWLDDINCYDRTDCVEXKXXPXVYFCCCEGNMCNEKFSYFPEMEVTQPTS

SEQ ID NO: 6 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 7 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 8 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 9 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 10 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 11 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 12 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 13 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 14 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 15 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 16 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 17 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 18 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 19 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 20 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 21 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 22 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 23 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 24 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 25 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 26 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 27 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 28 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 29 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 30 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 31 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 32 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 33 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 34 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 35 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 36 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 37 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 38 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 39 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 40 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 41 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 42 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 43 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 44 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 45 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 46 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 47 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 48 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 49 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 50 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 51 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 52 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 53 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 54 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 55 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 56 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 57 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 58 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 59 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 60 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 61 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 62 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 63 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 64 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 65 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 66 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 67 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 68 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 69 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 70 - protein, 109 aa, synthetic construct
  REGION 1..109: note = Synthetic Construct
GAILGRSETQECLYYNANWELERTNQTGVERCEGEQDKRLHCYATWRNISGSIEIVKKGCWLDDFNCYDRTDCVETEENPQVYFCCCEGNMCNEKFSYFPEMEVTQPTS

SEQ ID NO: 71 - protein, 111 aa, synthetic construct
  REGION 1..111: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNP

SEQ ID NO: 72 - protein, 115 aa, synthetic construct
  REGION 1..115: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNPVTPK

SEQ ID NO: 73 - protein, 109 aa, Homo sapiens
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 74 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 75 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 76 - protein, 109 aa, synthetic construct
  REGION 1..109: note = Synthetic Construct
GAILGRSETQECLFFNANWEKDRTNQTGVEPCYGDKDKRRHCFATWANISGSIEIVKQGCWLDDINCYDRTDCVEKKDSPEVYFCCCEGNMCNEKFSYFPEMEVTQPTS

SEQ ID NO: 77 - protein, 109 aa, synthetic construct
  REGION 1..109: note = Synthetic Construct
GAILGRSETQECLFFNANWAKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 78 - protein, 109 aa, synthetic construct
  REGION 1..109: note = Synthetic Construct
GAILGRSETQECLFFNANWEKDATNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 79 - protein, 109 aa, synthetic construct
  REGION 1..109: note = Synthetic Construct
GAILGRSETQECLFFNANWEKDRTNQTGVEPCYGDKAKRRHCFATWKNISGSIEIVKQGCWLDDINCYDRTDCVEKKDSPEVYFCCCEGNMCNEKFSYFPEMEVTQPTS

SEQ ID NO: 80 - protein, 109 aa, synthetic construct
  REGION 1..109: note = Synthetic Construct
GAILGRSETQECLFFNANWEKDRTNQTGVEPCYGDKDARRHCFATWKNISGSIEIVKQGCWLDDINCYDRTDCVEKKDSPEVYFCCCEGNMCNEKFSYFPEMEVTQPTS

SEQ ID NO: 81 - protein, 109 aa, synthetic construct
  REGION 1..109: note = Synthetic Construct
GAILGRSETQECLFFNANWEKDRTNQTGVEPCYGDKDKARHCFATWKNISGSIEIVKQGCWLDDINCYDRTDCVEKKDSPEVYFCCCEGNMCNEKFSYFPEMEVTQPTS

SEQ ID NO: 82 - protein, 109 aa, synthetic construct
  REGION 1..109: note = Synthetic Construct
GAILGRSETQECLFFNANWEKDRTNQTGVEPCYGDKDKRRHCFATWKNISGSIEIVAQGCWLDDINCYDRTDCVEKKDSPEVYFCCCEGNMCNEKFSYFPEMEVTQPTS

SEQ ID NO: 83 - protein, 109 aa, synthetic construct
  REGION 1..109: note = Synthetic Construct
GAILGRSETQECLFFNANWEKDRTNQTGVEPCYGDKDKRRHCFATWKNISGSIEIVYQGCWLDDINCYDRTDCVEKKDSPEVYFCCCEGNMCNEKFSYFPEMEVTQPTS

SEQ ID NO: 84 - protein, 109 aa, synthetic construct
  REGION 1..109: note = Synthetic Construct
GAILGRSETQECLFFNANWEKDRTNQTGVEPCYGDKDKRRHCFATWKNISGSIEIVFQGCWLDDINCYDRTDCVEKKDSPEVYFCCCEGNMCNEKFSYFPEMEVTQPTS

SEQ ID NO: 85 - protein, 109 aa, synthetic construct
  REGION 1..109: note = Synthetic Construct
GAILGRSETQECLFFNANWEKDRTNQTGVEPCYGDKDKRRHCFATWKNISGSIEIVIQGCWLDDINCYDRTDCVEKKDSPEVYFCCCEGNMCNEKFSYFPEMEVTQPTS

SEQ ID NO: 86 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCALDDINCYDRTDCVEKKDSPEVYFCCCEGNMCNEKFSYFPEMEVTQPTS

SEQ ID NO: 87 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWADDINCYDRTDCVEKKDSPEVYFCCCEGNMCNEKFSYFPEMEVTQPTS

SEQ ID NO: 88 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLKDINCYDRTDCVEKKDSPEVYFCCCEGNMCNEKFSYFPEMEVTQPTS

SEQ ID NO: 89 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLRDINCYDRTDCVEKKDSPEVYFCCCEGNMCNEKFSYFPEMEVTQPTS

SEQ ID NO: 90 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLADINCYDRTDCVEKKDSPEVYFCCCEGNMCNEKFSYFPEMEVTQPTS

SEQ ID NO: 91 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLFDINCYDRTDCVEKKDSPEVYFCCCEGNMCNEKFSYFPEMEVTQPTS

SEQ ID NO: 92 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLGDINCYDRTDCVEKKDSPEVYFCCCEGNMCNEKFSYFPEMEVTQPTS

SEQ ID NO: 93 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLMDINCYDRTDCVEKKDSPEVYFCCCEGNMCNEKFSYFPEMEVTQPTS

SEQ ID NO: 94 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLNDINCYDRTDCVEKKDSPEVYFCCCEGNMCNEKFSYFPEMEVTQPTS

SEQ ID NO: 95 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLIDINCYDRTDCVEKKDSPEVYFCCCEGNMCNEKFSYFPEMEVTQPTS

SEQ ID NO: 96 - protein, 109 aa, synthetic construct
  REGION 1..109: note = Synthetic Construct
GAILGRSETQECLFFNANWEKDRTNQTGVEPCYGDKDKRRHCFATWKNISGSIEIVKQGCWLDDANCYDRTDCVEKKDSPEVYFCCCEGNMCNEKFSYFPEMEVTQPTS

SEQ ID NO: 97 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 98 - protein, 4 aa, synthetic construct
  REGION 1..4: note = Synthetic Construct
GGGA

SEQ ID NO: 99 - protein, 4 aa, synthetic construct
  REGION 1..4: note = Synthetic Construct
GGGS

SEQ ID NO: 100 - protein, 4 aa, synthetic construct
  REGION 1..4: note = Synthetic Construct
GGGG

SEQ ID NO: 101 - protein, 5 aa, synthetic construct
  REGION 1..5: note = Synthetic Construct
GGGGA

SEQ ID NO: 102 - protein, 5 aa, synthetic construct
  REGION 1..5: note = Synthetic Construct
GGGGS

SEQ ID NO: 103 - protein, 5 aa, synthetic construct
  REGION 1..5: note = Synthetic Construct
GGGGG

SEQ ID NO: 104 - protein, 4 aa, synthetic construct
  REGION 1..4: note = Synthetic Construct
GGAG

SEQ ID NO: 105 - protein, 4 aa, synthetic construct
  REGION 1..4: note = Synthetic Construct
GGSG

SEQ ID NO: 106 - protein, 4 aa, synthetic construct
  REGION 1..4: note = Synthetic Construct
AGGG

SEQ ID NO: 107 - protein, 4 aa, synthetic construct
  REGION 1..4: note = Synthetic Construct
SGGG

SEQ ID NO: 108 - protein, 4 aa, synthetic construct
  REGION 1..4: note = Synthetic Construct
GAGA

SEQ ID NO: 109 - protein, 4 aa, synthetic construct
  REGION 1..4: note = Synthetic Construct
GSGS

SEQ ID NO: 110 - protein, 6 aa, synthetic construct
  REGION 1..6: note = Synthetic Construct
GAGAGA

SEQ ID NO: 111 - protein, 6 aa, synthetic construct
  REGION 1..6: note = Synthetic Construct
GSGSGS

SEQ ID NO: 112 - protein, 8 aa, synthetic construct
  REGION 1..8: note = Synthetic Construct
GAGAGAGA

SEQ ID NO: 113 - protein, 8 aa, synthetic construct
  REGION 1..8: note = Synthetic Construct
GSGSGSGS

SEQ ID NO: 114 - protein, 10 aa, synthetic construct
  REGION 1..10: note = Synthetic Construct
GAGAGAGAGA

SEQ ID NO: 115 - protein, 10 aa, synthetic construct
  REGION 1..10: note = Synthetic Construct
GSGSGSGSGS

SEQ ID NO: 116 - protein, 12 aa, synthetic construct
  REGION 1..12: note = Synthetic Construct
GAGAGAGAGAGA

SEQ ID NO: 117 - protein, 12 aa, synthetic construct
  REGION 1..12: note = Synthetic Construct
GSGSGSGSGSGS

SEQ ID NO: 118 - protein, 6 aa, synthetic construct
  REGION 1..6: note = Synthetic Construct
GGAGGA

SEQ ID NO: 119 - protein, 6 aa, synthetic construct
  REGION 1..6: note = Synthetic Construct
GGSGGS

SEQ ID NO: 120 - protein, 9 aa, synthetic construct
  REGION 1..9: note = Synthetic Construct
GGAGGAGGA

SEQ ID NO: 121 - protein, 9 aa, synthetic construct
  REGION 1..9: note = Synthetic Construct
GGSGGSGGS

SEQ ID NO: 122 - protein, 12 aa, synthetic construct
  REGION 1..12: note = Synthetic Construct
GGAGGAGGAGGA

SEQ ID NO: 123 - protein, 12 aa, synthetic construct
  REGION 1..12: note = Synthetic Construct
GGSGGSGGSGGS

SEQ ID NO: 124 - protein, 8 aa, synthetic construct
  REGION 1..8: note = Synthetic Construct
GGAGGGAG

SEQ ID NO: 125 - protein, 8 aa, synthetic construct
  REGION 1..8: note = Synthetic Construct
GGSGGGSG

SEQ ID NO: 126 - protein, 12 aa, synthetic construct
  REGION 1..12: note = Synthetic Construct
GGAGGGAGGGAG

SEQ ID NO: 127 - protein, 12 aa, synthetic construct
  REGION 1..12: note = Synthetic Construct
GGSGGGSGGGSG

SEQ ID NO: 128 - protein, 15 aa, synthetic construct
  REGION 1..15: note = Synthetic Construct
GGGGAGGGGAGGGGA

SEQ ID NO: 129 - protein, 15 aa, synthetic construct
  REGION 1..15: note = Synthetic Construct
GGGGSGGGGSGGGGS

SEQ ID NO: 130 - protein, 5 aa, synthetic construct
  REGION 1..5: note = Synthetic Construct
GGGAG

SEQ ID NO: 131 - protein, 6 aa, synthetic construct
  REGION 1..6: note = Synthetic Construct
GGGAGG

SEQ ID NO: 132 - protein, 7 aa, synthetic construct
  REGION 1..7: note = Synthetic Construct
GGGAGGG

SEQ ID NO: 133 - protein, 4 aa, synthetic construct
  REGION 1..4: note = Synthetic Construct
AAAL

SEQ ID NO: 134 - protein, 4 aa, synthetic construct
  REGION 1..4: note = Synthetic Construct
AAAK

SEQ ID NO: 135 - protein, 4 aa, synthetic construct
  REGION 1..4: note = Synthetic Construct
AAAR

SEQ ID NO: 136 - protein, 14 aa, synthetic construct
  REGION 1..14: note = Synthetic Construct
EGKSSGSGSESKST

SEQ ID NO: 137 - protein, 12 aa, synthetic construct
  REGION 1..12: note = Synthetic Construct
GSAGSAAGSGEF

SEQ ID NO: 138 - protein, 12 aa, synthetic construct
  REGION 1..12: note = Synthetic Construct
AEAAAKEAAAKA

SEQ ID NO: 139 - protein, 18 aa, synthetic construct
  REGION 1..18: note = Synthetic Construct
KESGSVSSEQLAQFRSLD

SEQ ID NO: 140 - protein, 10 aa, synthetic construct
  REGION 1..10: note = Synthetic Construct
GENLYFQSGG

SEQ ID NO: 141 - protein, 8 aa, synthetic construct
  REGION 1..8: note = Synthetic Construct
SACYCELS

SEQ ID NO: 142 - protein, 5 aa, synthetic construct
  REGION 1..5: note = Synthetic Construct
RSIAT

SEQ ID NO: 143 - protein, 17 aa, synthetic construct
  REGION 1..17: note = Synthetic Construct
RPACKIPNDLKQKVMNH

SEQ ID NO: 144 - protein, 36 aa, synthetic construct
  REGION 1..36: note = Synthetic Construct
GGSAGGSGSGSSGGSSGASGTGTAGGTGSGSGTGSG

SEQ ID NO: 145 - protein, 17 aa, synthetic construct
  REGION 1..17: note = Synthetic Construct
AAANSSIDLISVPVDSR

SEQ ID NO: 146 - protein, 36 aa, synthetic construct
  REGION 1..36: note = Synthetic Construct
GGSGGGSEGGGSEGGGSEGGGSEGGGSEGGGSGGGS

SEQ ID NO: 147 - protein, 5 aa, synthetic construct
  REGION 1..5: note = Synthetic Construct
EAAAK

SEQ ID NO: 148 - protein, 5 aa, synthetic construct
  REGION 1..5: note = Synthetic Construct
PAPAP

SEQ ID NO: 149 - protein, 109 aa, synthetic construct
  REGION 1..109: note = Synthetic Construct
GAILGRSETQECLFYNANWELERTNQTGVERCEGEKDKRLHCYATWRNISGSIEIVAKGCWLDDFNCYDRTDCVETEENPQVYFCCCEGNMCNEKFSYFPEMEVTQPTS

SEQ ID NO: 150 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151-153 - skipped (3 entries)

SEQ ID NO: 154 - protein, 6 aa, synthetic construct
  REGION 1..6: note = Synthetic Construct
NPVTPK

SEQ ID NO: 155 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 338 aa, synthetic construct
  REGION 1..338: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 157 - protein, 115 aa, synthetic construct
  REGION 1..115: note = Synthetic Construct
  VARIANT 11: note = MISC_FEATURE - Xaa is Ile or Leu
  VARIANT 12: note = MISC_FEATURE - Xaa is Phe or Tyr
  VARIANT 19: note = MISC_FEATURE - Xaa is Leu or Lys
  VARIANT 20: note = MISC_FEATURE - Xaa is Asp or Glu
  VARIANT 25: note = MISC_FEATURE - Xaa is Thr or Ser
  VARIANT 27: note = MISC_FEATURE - Xaa is Leu or Val
  VARIANT 29: note = MISC_FEATURE - Xaa is Pro or Arg
  VARIANT 31: note = MISC_FEATURE - Xaa is Tyr or Glu
  VARIANT 33: note = MISC_FEATURE - Xaa is Asp or Glu
  VARIANT 34: note = MISC_FEATURE - Xaa is Lys or Gln
  VARIANT 38: note = MISC_FEATURE - Xaa is Arg or Leu
  VARIANT 41: note = MISC_FEATURE - Xaa is Tyr or Phe
  VARIANT 45: note = MISC_FEATURE - Xaa is Arg or Lys
  VARIANT 47: note = MISC_FEATURE - Xaa is Ser or Ile
  VARIANT 48: note = MISC_FEATURE - Xaa is Ser or Thr
  VARIANT 50: note = MISC_FEATURE - Xaa is Ser or Thr
  VARIANT 51: note = MISC_FEATURE - Xaa is Ile or Leu
  VARIANT 53: note = MISC_FEATURE - Xaa is Ile or Leu
  VARIANT 56: note = MISC_FEATURE - Xaa is Lys or Gln
  VARIANT 63: note = MISC_FEATURE - Xaa is Phe or Ile
  VARIANT 69: note = MISC_FEATURE - Xaa is Gln, Thr, or Asp
  VARIANT 70: note = MISC_FEATURE - Xaa is Glu, Asp, or Thr
  VARIANT 74: note = MISC_FEATURE - Xaa is Lys or Thr
  VARIANT 75: note = MISC_FEATURE - Xaa is Lys or Glu
  VARIANT 76: note = MISC_FEATURE - Xaa is Asp or Glu
  VARIANT 77: note = MISC_FEATURE - Xaa is Ser or Asn
  VARIANT 79: note = MISC_FEATURE - Xaa is Glu or Gln
  VARIANT 89: note = MISC_FEATURE - Xaa is Phe or Met
GRGEAETRECXXYNANWEXXRTNQXGXEXCXGXXDKRXHCXASWXNXXGXXEXVKXGCWLDDXNCYDRXXCVAXXXXPXVYFCCCEGNXCNERFTHLPEAGGPEVTYEPPPTAPT

SEQ ID NO: 158 - protein, 115 aa, synthetic construct
  REGION 1..115: note = Synthetic Construct
GRGEAETRECIFYNANWEKDRTNQSGLEPCYGDQDKRRHCFASWKNSSGTIELVKQGCWLDDINCYDRQECVAKKDSPEVYFCCCEGNFCNERFTHLPEAGGPEVTYEPPPTAPT

SEQ ID NO: 159 - protein, 115 aa, synthetic construct
  REGION 1..115: note = Synthetic Construct
GRGEAETRECIYYNANWELDRTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 160 - protein, 115 aa, synthetic construct
  REGION 1..115: note = Synthetic Construct
GRGEAETRECIYYNANWELERTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 161 - protein, 115 aa, synthetic construct
  REGION 1..115: note = Synthetic Construct
GRGEAETRECIYYNANWELERTNQTGLERCEGEQDKRLHCYASWRNISGTIELVKKGCWLDDFNCYDRQECVAKKDSPEVYFCCCEGNFCNERFTHLPEAGGPEVTYEPPPTAPT

SEQ ID NO: 162 - protein, 115 aa, synthetic construct
  REGION 1..115: note = Synthetic Construct
GRGEAETRECIYYNANWELERTNQTGLERCEGEQDKRLHCYASWRNITGTIELVKKGCWLDDFNCYDRQECVAKKDSPEVYFCCCEGNFCNERFTHLPEAGGPEVTYEPPPTAPT

SEQ ID NO: 163 - protein, 115 aa, synthetic construct
  REGION 1..115: note = Synthetic Construct
GRGEAETRECIYYNANWELERTNQSGLEPCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 164 - protein, 115 aa, synthetic construct
  REGION 1..115: note = Synthetic Construct
GRGEAETRECIYYNANWELERTNQSGLERCYGDKDKRLHCYASWRNSSGTIELVKKGCWLDDFNCYDRQECVAKKDSPEVYFCCCEGNFCNERFTHLPEAGGPEVTYEPPPTAPT

SEQ ID NO: 165 - protein, 115 aa, synthetic construct
  REGION 1..115: note = Synthetic Construct
GRGEAETRECIYYNANWELERTNQSGLERCEGEQDKRLHCFASWKNSSGTIELVKQGCWLDDFNCYDRQECVAKKDSPEVYFCCCEGNFCNERFTHLPEAGGPEVTYEPPPTAPT

SEQ ID NO: 166 - protein, 115 aa, synthetic construct
  REGION 1..115: note = Synthetic Construct
GRGEAETRECIFYNANWEKDRTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 167 - protein, 115 aa, synthetic construct
  REGION 1..115: note = Synthetic Construct
GRGEAETRECIYYNANWELERTNQSGLERCYGDQDKRRHCYASWRNSSGTIELVKKGCWLDDFNCYDRQECVAKKDSPEVYFCCCEGNFCNERFTHLPEAGGPEVTYEPPPTAPT

SEQ ID NO: 168 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRTDCVATEENPQVYFCCCEGNMCNERFTHLPEAGGPEVTYEPPPTAPT

SEQ ID NO: 169 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRDTCVATEENPQVYFCCCEGNMCNERFTHLPEAGGPEVTYEPPPTAPT

SEQ ID NO: 170 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRTDCVATKENPQVYFCCCEGNMCNERFTHLPEAGGPEVTYEPPPTAPT

SEQ ID NO: 171 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPT

SEQ ID NO: 172 - protein, 345 aa, synthetic construct
  REGION 1..345: note = Synthetic Construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 174 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 175 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 176 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 177 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 178 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 179 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 180 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 181 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 182 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 183 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 184 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 185 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 186 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 187 - protein, 107 aa, synthetic construct
  REGION 1..107: note = Synthetic Construct
  VARIANT 35: note = MISC_FEATURE - Xaa is Asp or Arg
  VARIANT 63: note = MISC_FEATURE - Xaa is Ile, Phe, Glu, Asp, Tyr, Ser, Asn, Gln, or Thr
  VARIANT 64: note = MISC_FEATURE - Xaa is Asn or Thr
  VARIANT 73: note = MISC_FEATURE - Xaa is Ala or Glu
  VARIANT 74: note = MISC_FEATURE - Xaa is Thr or Lys
  VARIANT 75: note = MISC_FEATURE - Xaa is Glu or Lys
  VARIANT 76: note = MISC_FEATURE - Xaa is Glu or Asp
  VARIANT 77: note = MISC_FEATURE - Xaa is Asn or Ser
  VARIANT 79: note = MISC_FEATURE - Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 188 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 189 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 190 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 191 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 192 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 193 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 194 - protein, 109 aa, synthetic construct
  REGION 1..109: note = Synthetic Construct
  VARIANT 37: note = MISC_FEATURE - Xaa is Asp or Arg
  VARIANT 65: note = MISC_FEATURE - Xaa is Ile, Phe, Glu, Asp, Tyr, Ser, Asn, Gln, or Thr
  VARIANT 66: note = MISC_FEATURE - Xaa is Asn or Thr
  VARIANT 75: note = MISC_FEATURE - Xaa is Ala or Glu
  VARIANT 76: note = MISC_FEATURE - Xaa is Thr or Lys
  VARIANT 77: note = MISC_FEATURE - Xaa is Glu or Lys
  VARIANT 78: note = MISC_FEATURE - Xaa is Glu or Asp
  VARIANT 79: note = MISC_FEATURE - Xaa is Asn or Ser
  VARIANT 81: note = MISC_FEATURE - Xaa is Gln, Glu, Lys, Arg, Asp, or Asn
GAILGRSETQECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 195 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 196 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 197 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 198 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 199 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDFNCYDRTDCVATEENPQVYFCCCEGNMCNEKFSYFPEMEVTQPTS

SEQ ID NO: 200 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVEKKDSPEVYFCCCEGNMCNEKFSYFPEMEVTQPTS

SEQ ID NO: 201 - protein, 107 aa, synthetic construct
  REGION 1..107: note = Synthetic Construct
GRGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 202 - protein, 107 aa, synthetic construct
  REGION 1..107: note = Synthetic Construct
GRGEAETRECIYYNANWELERTNQSGLERCEGEQDKRLHCFATWKNISGSIEIVKQGCWLDDFNCYDRTDCVEKKDSPQVYFCCCEGNMCNEKFSYFPEMEVTQPTS

SEQ ID NO: 203 - protein, 107 aa, synthetic construct
  REGION 1..107: note = Synthetic Construct
GRGEAETRECIYYNANWELERTNQSGLERCEGEQDKRLHCYASWKNISGSIEIVKQGCWLDDFNCYDRTDCVEKKDSPQVYFCCCEGNMCNEKFSYFPEMEVTQPTS

SEQ ID NO: 204 - protein, 107 aa, synthetic construct
  REGION 1..107: note = Synthetic Construct
GRGEAETRECIYYNANWELERTNQSGLERCEGEQDKRLHCYASWRNSSGSIEIVKQGCWLDDFNCYDRTDCVEKKDSPQVYFCCCEGNMCNEKFSYFPEMEVTQPTS

SEQ ID NO: 205 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRTDCVEKKDSPQVYFCCCEGNMCNEKFSYFPEMEVTQPTS

SEQ ID NO: 206 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKDSPQVYFCCCEGNMCNEKFSYFPEMEVTQPTS

SEQ ID NO: 207 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATEENPQVYFCCCEGNMCNEKFSYFPEMEVTQPTS

SEQ ID NO: 208 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDNNCYDRTDCVEKKDSPQVYFCCCEGNMCNEKFSYFPEMEVTQPTS

SEQ ID NO: 209 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDTNCYDRTDCVEKKDSPQVYFCCCEGNMCNEKFSYFPEMEVTQPTS

SEQ ID NO: 210 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 211 - protein, 107 aa, synthetic construct
  REGION 1..107: note = Synthetic Construct
GRGEAETRECIYYNANWELERTNQSGLERCEGEQRKRLHCYASWRNSSGTIEIVKQGCWLDDFNCYDRTDCVEKKDSPQVYFCCCEGNMCNEKFSYFPEMEVTQPTS

SEQ ID NO: 212 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVETKDSPQVYFCCCEGNMCNEKFSYFPEMEVTQPTS

SEQ ID NO: 213 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 214 - protein, 109 aa, synthetic construct
  REGION 1..109: note = Synthetic Construct
GAILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 215 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 216 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 217 - protein, 339 aa, synthetic construct
  REGION 1..339: note = Synthetic Construct
GAILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 337 aa, synthetic construct
  REGION 1..337: note = Synthetic Construct
GRGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 337 aa, synthetic construct
  REGION 1..337: note = Synthetic Construct
GRGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 337 aa, synthetic construct
  REGION 1..337: note = Synthetic Construct
GRGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 107 aa, synthetic construct
  REGION 1..107: note = Synthetic Construct
ILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 222 - protein, 106 aa, synthetic construct
  REGION 1..106: note = Synthetic Construct
RGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 223 - protein, 106 aa, synthetic construct
  REGION 1..106: note = Synthetic Construct
RGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 224 - protein, 106 aa, synthetic construct
  REGION 1..106: note = Synthetic Construct
RGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 225 - protein, 106 aa, synthetic construct
  REGION 1..106: note = Synthetic Construct
LGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 226 - protein, 105 aa, synthetic construct
  REGION 1..105: note = Synthetic Construct
GEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 227 - protein, 105 aa, synthetic construct
  REGION 1..105: note = Synthetic Construct
GEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 228 - protein, 105 aa, synthetic construct
  REGION 1..105: note = Synthetic Construct
GEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 229 - protein, 105 aa, synthetic construct
  REGION 1..105: note = Synthetic Construct
GRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 230 - protein, 104 aa, synthetic construct
  REGION 1..104: note = Synthetic Construct
EAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 231 - protein, 104 aa, synthetic construct
  REGION 1..104: note = Synthetic Construct
EAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 232 - protein, 104 aa, synthetic construct
  REGION 1..104: note = Synthetic Construct
EAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 233 - protein, 104 aa, synthetic construct
  REGION 1..104: note = Synthetic Construct
RAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 234 - protein, 103 aa, synthetic construct
  REGION 1..103: note = Synthetic Construct
AETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 235 - protein, 103 aa, synthetic construct
  REGION 1..103: note = Synthetic Construct
AETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 236 - protein, 103 aa, synthetic construct
  REGION 1..103: note = Synthetic Construct
AETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 237 - protein, 103 aa, synthetic construct
  REGION 1..103: note = Synthetic Construct
AETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 238 - protein, 102 aa, synthetic construct
  REGION 1..102: note = Synthetic Construct
ETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 239 - protein, 102 aa, synthetic construct
  REGION 1..102: note = Synthetic Construct
ETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 240 - protein, 102 aa, synthetic construct
  REGION 1..102: note = Synthetic Construct
ETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 241 - protein, 102 aa, synthetic construct
  REGION 1..102: note = Synthetic Construct
ETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 242 - protein, 101 aa, synthetic construct
  REGION 1..101: note = Synthetic Construct
TRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 243 - protein, 101 aa, synthetic construct
  REGION 1..101: note = Synthetic Construct
TRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 244 - protein, 101 aa, synthetic construct
  REGION 1..101: note = Synthetic Construct
TRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 245 - protein, 101 aa, synthetic construct
  REGION 1..101: note = Synthetic Construct
TRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 246 - protein, 100 aa, synthetic construct
  REGION 1..100: note = Synthetic Construct
RECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 247 - protein, 100 aa, synthetic construct
  REGION 1..100: note = Synthetic Construct
RECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 248 - protein, 100 aa, synthetic construct
  REGION 1..100: note = Synthetic Construct
RECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 249 - protein, 100 aa, synthetic construct
  REGION 1..100: note = Synthetic Construct
RECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 250 - protein, 106 aa, synthetic construct
  REGION 1..106: note = Synthetic Construct
RGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 251 - protein, 106 aa, synthetic construct
  REGION 1..106: note = Synthetic Construct
RGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 252 - protein, 107 aa, synthetic construct
  REGION 1..107: note = Synthetic Construct
ILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 253 - protein, 107 aa, synthetic construct
  REGION 1..107: note = Synthetic Construct
ILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 254 - protein, 106 aa, synthetic construct
  REGION 1..106: note = Synthetic Construct
RGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 255 - protein, 106 aa, synthetic construct
  REGION 1..106: note = Synthetic Construct
RGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 256 - protein, 105 aa, synthetic construct
  REGION 1..105: note = Synthetic Construct
GEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 257 - protein, 105 aa, synthetic construct
  REGION 1..105: note = Synthetic Construct
GEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 258 - protein, 106 aa, synthetic construct
  REGION 1..106: note = Synthetic Construct
LGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 259 - protein, 106 aa, synthetic construct
  REGION 1..106: note = Synthetic Construct
LGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 260 - protein, 105 aa, synthetic construct
  REGION 1..105: note = Synthetic Construct
GEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 261 - protein, 105 aa, synthetic construct
  REGION 1..105: note = Synthetic Construct
GEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 262 - protein, 104 aa, synthetic construct
  REGION 1..104: note = Synthetic Construct
EAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 263 - protein, 104 aa, synthetic construct
  REGION 1..104: note = Synthetic Construct
EAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 264 - protein, 105 aa, synthetic construct
  REGION 1..105: note = Synthetic Construct
GRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 265 - protein, 105 aa, synthetic construct
  REGION 1..105: note = Synthetic Construct
GRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 266 - protein, 104 aa, synthetic construct
  REGION 1..104: note = Synthetic Construct
EAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 267 - protein, 104 aa, synthetic construct
  REGION 1..104: note = Synthetic Construct
EAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 268 - protein, 103 aa, synthetic construct
  REGION 1..103: note = Synthetic Construct
AETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 269 - protein, 103 aa, synthetic construct
  REGION 1..103: note = Synthetic Construct
AETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 270 - protein, 104 aa, synthetic construct
  REGION 1..104: note = Synthetic Construct
RAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 271 - protein, 104 aa, synthetic construct
  REGION 1..104: note = Synthetic Construct
RSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 272 - protein, 103 aa, synthetic construct
  REGION 1..103: note = Synthetic Construct
AETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 273 - protein, 103 aa, synthetic construct
  REGION 1..103: note = Synthetic Construct
AETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 274 - protein, 102 aa, synthetic construct
  REGION 1..102: note = Synthetic Construct
ETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 275 - protein, 102 aa, synthetic construct
  REGION 1..102: note = Synthetic Construct
ETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 276 - protein, 103 aa, synthetic construct; identical to SEQ ID NO: 236
  REGION 1..103: note = Synthetic Construct

SEQ ID NO: 277 - protein, 103 aa, synthetic construct
  REGION 1..103: note = Synthetic Construct
SETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 278 - protein, 102 aa, synthetic construct
  REGION 1..102: note = Synthetic Construct
ETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 279 - protein, 102 aa, synthetic construct
  REGION 1..102: note = Synthetic Construct
ETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 280 - protein, 101 aa, synthetic construct
  REGION 1..101: note = Synthetic Construct
TRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 281 - protein, 101 aa, synthetic construct
  REGION 1..101: note = Synthetic Construct
TRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 282 - protein, 102 aa, synthetic construct; identical to SEQ ID NO: 240
  REGION 1..102: note = Synthetic Construct

SEQ ID NO: 283 - protein, 102 aa, synthetic construct
  REGION 1..102: note = Synthetic Construct
ETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 284 - protein, 101 aa, synthetic construct
  REGION 1..101: note = Synthetic Construct
TRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 285 - protein, 101 aa, synthetic construct
  REGION 1..101: note = Synthetic Construct
TRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 286 - protein, 100 aa, synthetic construct
  REGION 1..100: note = Synthetic Construct
RECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 287 - protein, 100 aa, synthetic construct
  REGION 1..100: note = Synthetic Construct
RECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 288 - protein, 101 aa, synthetic construct; identical to SEQ ID NO: 244
  REGION 1..101: note = Synthetic Construct

SEQ ID NO: 289 - protein, 101 aa, synthetic construct
  REGION 1..101: note = Synthetic Construct
TQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 290 - protein, 100 aa, synthetic construct
  REGION 1..100: note = Synthetic Construct
RECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 291 - protein, 100 aa, synthetic construct
  REGION 1..100: note = Synthetic Construct
RECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 292 - protein, 100 aa, synthetic construct; identical to SEQ ID NO: 248
  REGION 1..100: note = Synthetic Construct

SEQ ID NO: 293 - protein, 100 aa, synthetic construct
  REGION 1..100: note = Synthetic Construct
QECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 294 - protein, 337 aa, synthetic construct
  REGION 1..337: note = Synthetic Construct
ILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - protein, 336 aa, synthetic construct
  REGION 1..336: note = Synthetic Construct
RGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 296 - protein, 336 aa, synthetic construct
  REGION 1..336: note = Synthetic Construct
RGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - protein, 336 aa, synthetic construct
  REGION 1..336: note = Synthetic Construct
RGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 336 aa, synthetic construct
  REGION 1..336: note = Synthetic Construct
LGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 335 aa, synthetic construct
  REGION 1..335: note = Synthetic Construct
GEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 335 aa, synthetic construct
  REGION 1..335: note = Synthetic Construct
GEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 335 aa, synthetic construct
  REGION 1..335: note = Synthetic Construct
GEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 335 aa, synthetic construct
  REGION 1..335: note = Synthetic Construct
GRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 334 aa, synthetic construct
  REGION 1..334: note = Synthetic Construct
EAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 334 aa, synthetic construct
  REGION 1..334: note = Synthetic Construct
EAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 334 aa, synthetic construct
  REGION 1..334: note = Synthetic Construct
EAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 334 aa, synthetic construct
  REGION 1..334: note = Synthetic Construct
RAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 333 aa, synthetic construct
  REGION 1..333: note = Synthetic Construct
AETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 333 aa, synthetic construct
  REGION 1..333: note = Synthetic Construct
AETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 333 aa, synthetic construct
  REGION 1..333: note = Synthetic Construct
AETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 333 aa, synthetic construct
  REGION 1..333: note = Synthetic Construct
AETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 332 aa, synthetic construct
  REGION 1..332: note = Synthetic Construct
ETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 332 aa, synthetic construct
  REGION 1..332: note = Synthetic Construct
ETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 332 aa, synthetic construct
  REGION 1..332: note = Synthetic Construct
ETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 332 aa, synthetic construct
  REGION 1..332: note = Synthetic Construct
ETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 331 aa, synthetic construct
  REGION 1..331: note = Synthetic Construct
TRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 331 aa, synthetic construct
  REGION 1..331: note = Synthetic Construct
TRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 331 aa, synthetic construct
  REGION 1..331: note = Synthetic Construct
TRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 331 aa, synthetic construct
  REGION 1..331: note = Synthetic Construct
TRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 330 aa, synthetic construct
  REGION 1..330: note = Synthetic Construct
RECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 330 aa, synthetic construct
  REGION 1..330: note = Synthetic Construct
RECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 330 aa, synthetic construct
  REGION 1..330: note = Synthetic Construct
RECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 330 aa, synthetic construct
  REGION 1..330: note = Synthetic Construct
RECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 337 aa, synthetic construct
  REGION 1..337: note = Synthetic Construct
GRGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 337 aa, synthetic construct
  REGION 1..337: note = Synthetic Construct
GRGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 339 aa, synthetic construct
  REGION 1..339: note = Synthetic Construct
GAILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 339 aa, synthetic construct
  REGION 1..339: note = Synthetic Construct
GAILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 337 aa, synthetic construct
  REGION 1..337: note = Synthetic Construct
GRGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 337 aa, synthetic construct
  REGION 1..337: note = Synthetic Construct
GRGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 336 aa, synthetic construct
  REGION 1..336: note = Synthetic Construct
RGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 336 aa, synthetic construct
  REGION 1..336: note = Synthetic Construct
RGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 337 aa, synthetic construct
  REGION 1..337: note = Synthetic Construct
ILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 337 aa, synthetic construct
  REGION 1..337: note = Synthetic Construct
ILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 336 aa, synthetic construct
  REGION 1..336: note = Synthetic Construct
RGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 336 aa, synthetic construct
  REGION 1..336: note = Synthetic Construct
RGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 335 aa, synthetic construct
  REGION 1..335: note = Synthetic Construct
GEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 335 aa, synthetic construct
  REGION 1..335: note = Synthetic Construct
GEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 336 aa, synthetic construct
  REGION 1..336: note = Synthetic Construct
LGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 336 aa, synthetic construct
  REGION 1..336: note = Synthetic Construct
LGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 335 aa, synthetic construct
  REGION 1..335: note = Synthetic Construct
GEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - protein, 335 aa, synthetic construct
  REGION 1..335: note = Synthetic Construct
GEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 334 aa, synthetic construct
  REGION 1..334: note = Synthetic Construct
EAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 334 aa, synthetic construct
  REGION 1..334: note = Synthetic Construct
EAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 335 aa, synthetic construct
  REGION 1..335: note = Synthetic Construct
GRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 335 aa, synthetic construct
  REGION 1..335: note = Synthetic Construct
GRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 334 aa, synthetic construct
  REGION 1..334: note = Synthetic Construct
EAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 334 aa, synthetic construct
  REGION 1..334: note = Synthetic Construct
EAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 333 aa, synthetic construct
  REGION 1..333: note = Synthetic Construct
AETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 333 aa, synthetic construct
  REGION 1..333: note = Synthetic Construct
AETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 334 aa, synthetic construct
  REGION 1..334: note = Synthetic Construct
RAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 334 aa, synthetic construct
  REGION 1..334: note = Synthetic Construct
RSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 333 aa, synthetic construct
  REGION 1..333: note = Synthetic Construct
AETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 333 aa, synthetic construct
  REGION 1..333: note = Synthetic Construct
AETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 332 aa, synthetic construct
  REGION 1..332: note = Synthetic Construct
ETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 332 aa, synthetic construct
  REGION 1..332: note = Synthetic Construct
ETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 333 aa, synthetic construct; identical to SEQ ID NO: 309
  REGION 1..333: note = Synthetic Construct

SEQ ID NO: 356 - protein, 333 aa, synthetic construct
  REGION 1..333: note = Synthetic Construct
SETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 332 aa, synthetic construct
  REGION 1..332: note = Synthetic Construct
ETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 332 aa, synthetic construct
  REGION 1..332: note = Synthetic Construct
ETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 331 aa, synthetic construct
  REGION 1..331: note = Synthetic Construct
TRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 331 aa, synthetic construct
  REGION 1..331: note = Synthetic Construct
TRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 330 aa, synthetic construct
  REGION 1..330: note = Synthetic Construct
QECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 332 aa, synthetic construct; identical to SEQ ID NO: 313
  REGION 1..332: note = Synthetic Construct

SEQ ID NO: 363 - protein, 332 aa, synthetic construct
  REGION 1..332: note = Synthetic Construct
ETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 331 aa, synthetic construct
  REGION 1..331: note = Synthetic Construct
TRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 331 aa, synthetic construct
  REGION 1..331: note = Synthetic Construct
TRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 330 aa, synthetic construct
  REGION 1..330: note = Synthetic Construct
RECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 330 aa, synthetic construct
  REGION 1..330: note = Synthetic Construct
RECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 331 aa, synthetic construct; identical to SEQ ID NO: 317
  REGION 1..331: note = Synthetic Construct

SEQ ID NO: 369 - protein, 331 aa, synthetic construct
  REGION 1..331: note = Synthetic Construct
TQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 330 aa, synthetic construct
  REGION 1..330: note = Synthetic Construct
RECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 330 aa, synthetic construct
  REGION 1..330: note = Synthetic Construct
RECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 330 aa, synthetic construct; identical to SEQ ID NO: 321
  REGION 1..330: note = Synthetic Construct

SEQ ID NO: 373 - protein, 337 aa, synthetic construct
  REGION 1..337: note = Synthetic Construct
GRGEAETRECIYYNANWELERTNQSGLERCEGEQRKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 374 - protein, 4 aa, synthetic construct
  REGION 1..4: note = Synthetic Construct
TEEN

SEQ ID NO: 375 - protein, 4 aa, synthetic construct
  REGION 1..4: note = Synthetic Construct
TKEN

SEQ ID NO: 376 - protein, 4 aa, synthetic construct
KKDS

SEQ ID NO: 377 - protein, 4 aa, synthetic construct
TKDS

SEQ ID NO: 378 - protein, 10 aa, synthetic construct
  VARIANT 1: note = Any amino acid
  VARIANT 3: note = Any amino acid
  VARIANT 5: note = Any amino acid
  VARIANT 7: note = Any amino acid
  VARIANT 9: note = Any amino acid
  VARIANT 1..10: note = This sequence may encompass 1-5 XP repeating units
XPXPXPXPXP

(19) United States
(12) Patent Application Publication
(10) Pub. No.: US 2026/0125452 A1
(43) Pub. Date: May 7, 2026

(54) METHODS OF USING ACTIVIN RECEPTOR TYPE II VARIANTS

(71) Applicant: Keros Therapeutics, Inc., Lexington, MA (US)

(72) Inventors: Jasbir S. SEEHRA, Lexington, MA (US); Jennifer LACHEY, Lincoln, MA (US)

(21) Appl. No.: 19/390,938

(22) Filed: Nov. 17, 2025

Related U.S. Application Data
(63) Continuation of application No. PCT/US2024/029937, filed on May 17, 2024.
(60) Provisional application No. 63/467,474, filed on May 18, 2023.
(60) Provisional application No. 63/530,575, filed on Aug. 3, 2023.
(60) Provisional application No. 63/538,905, filed on Sep. 18, 2023.
(60) Provisional application No. 63/547,741, filed on Nov. 8, 2023.
(60) Provisional application No. 63/608,017, filed on Dec. 8, 2023.

(51) Int. Cl. C07K 14/71; A61K 38/00; A61K 45/06; A61P 9/04

(52) U.S. Cl. CPC C07K 14/71; A61K 45/06; A61P 9/04; A61K 38/00

(57) ABSTRACT

The invention features methods for the treatment of a cardiovascular disease using a polypeptide containing an extracellular ActRII variant, such as an ActRIIA variant, ActRIIB variant, or ActRII chimera. The polypeptide may include an extracellular ActRII variant fused to an Fc domain monomer. Treatment of a cardiovascular disease with a polypeptide described herein may reduce fibrosis, reduce inflammation, reduce cardiac remodeling, improve cardiac function, improve ventricular function, or slow or inhibit disease progression, which may reduce or delay the need for coronary revascularization, heart valve repair or replacement, implantation of a cardioverter-defibrillator, cardiac resynchronization therapy, a ventricular assist device, or a heart transplant.

### SEQUENCE LISTING

The instant application contains a Sequence Listing which has been submitted electronically in XML file format and is hereby incorporated by reference in its entirety. Said XML copy, created on Nov. 12, 2025, is named 51184-052007_Sequence_Listing_11_12_25.xml and is 641,657 bytes in size.

### BACKGROUND OF THE INVENTION

Cardiovascular diseases (CVDs) are a group of disorders of the heart and blood vessels and are the number one cause of death globally, taking an estimated 17.9 million lives each year. Persistent inflammation and fibrosis are observed in many cardiovascular diseases, such as heart failure, and are also upregulated as part of normal aging. Failure to resolve inflammation and altered immune responses underlie the pathology of CVDs. Accordingly, therapies that reduce inflammation and fibrosis may be effective in treating or preventing the development of cardiovascular diseases. Given that cardiovascular diseases are a leading cause of death throughout the world, there remains a need for therapies that can be used to prevent or treat CVDs.

### SUMMARY OF THE INVENTION

The present invention features polypeptides that include an extracellular activin receptor type II (ActRII) variant, such as an extracellular ActRIIA variant, an extracellular ActRIIB variant, or an extracellular ActRII chimera. In some embodiments, a polypeptide of the invention includes an extracellular ActRII variant fused to the N- or C-terminus of an Fc domain monomer (e.g., by fusion of the C-terminus of the ActRII variant to the N-terminus of an Fc domain monomer by way of a linker), which may be attached by amino acid or other covalent bonds and increase stability of the polypeptide. A polypeptide including an extracellular ActRII variant fused to an Fc domain monomer may also form a dimer (e.g., a homodimer or heterodimer) through the interaction between two Fc domain monomers. The polypeptides of the invention may be used to treat a subject having or at risk of developing a cardiovascular disease, such as cardiomyopathy, a calcification disease, heart valve disease, vasculitis, deep vein thrombosis, left-sided heart failure (e.g., heart failure with reduced ejection fraction (HFrEF) or heart failure with preserved ejection fraction (HFpEF), such as chronic HFrEF or chronic HFpEF), right-sided heart failure, backward heart failure, forward heart failure, high-output heart failure, low-output heart failure, compensated heart failure, decompensated heart failure, ventricular dysfunction (e.g., left ventricular dysfunction), an arrythmia, or an aneurysm. The polypeptides of the invention may prevent the cardiovascular disease, delay the development of the cardiovascular disease, slow the progression of the cardiovascular disease, or reduce cardiac remodeling, fibrosis, or inflammation associated with the cardiovascular disease. In some embodiments, the polypeptides of the invention reduce cardiovascular risk (e.g., in a subject being treated for another disease or condition, such as a disease or condition associated with increased risk of developing a cardiovascular disease or death due to a cardiovascular disease, such as a myelodysplastic syndrome (MDS)).

Exemplary embodiments of the invention are described in the enumerated paragraphs below.
E1. A method of treating a subject having a or at risk of developing a calcification disease, the method comprising administering to the subject a therapeutically effective amount of a composition of Table 10, a composition of Table 11, or a composition of Table 12.
E2. A method of treating a subject having or at risk of developing cardiomyopathy, the method comprising administering to the subject a therapeutically effective amount of a composition of Table 10, a composition of Table 11, or a composition of Table 12.
E3. A method of treating a subject having or at risk of developing right-sided heart failure, left-sided heart failure, backward heart failure, forward heart failure, high-output heart failure, low-output heart failure, compensated heart failure, or decompensated heart failure, the method comprising administering to the subject a therapeutically effective amount of a composition of Table 10, a composition of Table 11, or a composition of Table 12.
E4. A method of treating a subject having or at risk of developing a heart valve disease, the method comprising administering to the subject a therapeutically effective amount of a composition of Table 10, a composition of Table 11, or a composition of Table 12.
E5. A method of treating a subject having or at risk of developing an arrhythmia, the method comprising administering to the subject a therapeutically effective amount of a composition of Table 10, a composition of Table 11, or a composition of Table 12.
E6. A method of treating a subject having or at risk of developing hyperlipidemia or hyperlipoproteinemia, the method comprising administering to the subject a therapeutically effective amount of a composition of Table 10, a composition of Table 11, or a composition of Table 12.
E7. A method of treating a subject having or at risk of developing vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia, the method comprising administering to the subject a therapeutically effective amount of a composition of Table 10, a composition of Table 11, or a composition of Table 12.
E8. A method of treating a subject having or at risk of developing ventricular dysfunction, the method comprising administering to the subject a therapeutically effective amount of a composition of Table 10, a composition of Table 11, or a composition of Table 12.
E9. A method of reducing inflammation in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia, the method comprising administering to the subject a therapeutically effective amount of a composition of Table 10, a composition of Table 11, or a composition of Table 12.
E10. A method of reducing cardiac remodeling in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia, the method comprising administering to the subject a therapeutically effective amount of a composition of Table 10, a composition of Table 11, or a composition of Table 12.
E11. A method of reducing fibrosis in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia, the method comprising administering to the subject a therapeutically effective amount of a composition of Table 10, a composition of Table 11, or a composition of Table 12.
E12. A method of improving cardiac or ventricular function in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia, the method comprising administering to the subject a therapeutically effective amount of a composition of Table 10, a composition of Table 11, or a composition of Table 12.
E13. A method of slowing or inhibiting progression of a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof, the method comprising administering to the subject a therapeutically effective amount of a composition of Table 10, a composition of Table 11, or a composition of Table 12.
E14. A method of delaying development of or preventing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof, the method comprising administering to the subject a therapeutically effective amount of a composition of Table 10, a composition of Table 11, or a composition of Table 12.
E15. A method of reversing progression of a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof, the method comprising administering to the subject a therapeutically effective amount of a composition of Table 10, a composition of Table 11, or a composition of Table 12.
E16. A method of decreasing NT-proBNP (e.g., from Baseline measurements) in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof, the method comprising administering to the subject a therapeutically effective amount of a composition of Table 10, a composition of Table 11, or a composition of Table 12.
E17. A method of improving cardiopulmonary exercise testing (CPET) parameters (e.g., respiratory gas exchange, heart rate, blood pressure, or other physiological variables assessed by cardiopulmonary exercise test, such as a test using a cycle ergometer or treadmill, e.g., from Baseline measurements) in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof, the method comprising administering to the subject a therapeutically effective amount of a composition of Table 10, a composition of Table 11, or a composition of Table 12.
E18. A method of improving echocardiographic (ECHO) parameters (e.g., left ventricular ejection fraction (LVEF) or other cardiac function variables measured by echocardiogram, e.g., from Baseline measurements) in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof, the method comprising administering to the subject a therapeutically effective amount of a composition of Table 10, a composition of Table 11, or a composition of Table 12.
E19. A method of preventing or reducing aneurysm formation in a subject in need thereof, the method comprising administering to the subject a therapeutically effective amount of a composition of Table 10, a composition of Table 11, or a composition of Table 12.
E20. The method of any one of E1 and E9-E18, wherein the calcification disease is Monckeberg's vascular calcification disease, vascular calcification, or valvular calcification.
E21. The method of any one of E2 and E9-E18, wherein the cardiomyopathy is dilated cardiomyopathy, hypertrophic cardiomyopathy, restrictive cardiomyopathy, arrhythmogenic right ventricular dysplasia, unclassified cardiomyopathy, pediatric cardiomyopathy, or peripartum cardiomyopathy.
E22. The method of any one of E3 and E9-E18, wherein the left-sided heart failure is heart failure with reduced ejection fraction (HFrEF) or heart failure with preserved ejection fraction (HFpEF).
E23. The method of E22, wherein the left-sided heart failure is HFrEF.
E24. The method of E22, wherein the left-sided heart failure is HFpEF.
E25. The method of E23, wherein the HFpEF is HFpEF with defined ≥Grade 2 diastolic dysfunction.
E26. The method of any one of E3, E9-E18, and E22-E25, wherein the heart failure is chronic.
E27. The method of any one of E3, E9-E18, and E22-E25, wherein the heart failure is acute.
E28. The method of any one of E3, E9-E18, and E22-E27, wherein the subject has New York Heart Association (NYHA) Functional Class II to IV symptoms.
E29. The method of E28, wherein the subject has NHYA Functional Class II symptoms.
E30. The method of E28, wherein the subject has NHYA Functional Class III symptoms.
E31. The method of E28, wherein the subject has NHYA Functional Class IV symptoms.
E32. The method of any one of E4 and E9-E18, wherein the heart valve disease is aortic stenosis, mitral valve insufficiency, mitral valve prolapse, or rheumatic heart disease.
E33. The method of any one of E5 and E9-E18, wherein the arrythmia is atrial flutter or atrial fibrillation, a ventricular arrythmia, cardiac arrhythmia associated with myocardial ischemia or myocardial infarction, or arrhythmia associated with cerebral hemorrhage.
E34. The method of any one of E6 and E9-E18, wherein the hyperlipidemia or hyperlipoproteinemia is congenital hyperlipidemia or hyperlipoproteinemia.
E35. The method of E34, wherein the congenital hyperlipidemia or hyperlipoproteinemia is familial combined hyperlipidemia (FCHL), primary hyperlipoproteinemia, hyperapobetalipoproteinemia, Frederickson type Ill hyperlipidemia (familial dysbetalipoprotenemia), Frederickson type IV hyperlipidemia (familial hypertriglyceridemia), Frederickson type V hyperlipidemia (endogenous hypertriglyceridemia), or small dense LDL syndrome (LDL phenotype B).
E36. The method of any one of E6 and E9-E18, wherein the hyperlipidemia or hyperlipoproteinemia is acquired hyperlipidemia or hyperlipoproteinemia.
E37. The method of any one of E8 and E9-E18, wherein the ventricular dysfunction is left ventricular dysfunction.
E38. The method of any one of E1-E37, wherein the subject has elevated N-terminal pro b-type natriuretic peptide (NT-proBNP) (>500 pg/mL).
E39. The method of any one of E1-E38, wherein the method further comprises administering a Heart Failure (HF) background therapy.
E40. The method of any one of E1-E38, wherein the subject is on a HF background therapy (e.g., taking a HF background therapy prior to treatment initiation with a composition of Table 10, Table 11, or Table 12).
E41. The method of E39 or E40, wherein the HF background therapy is a sodium-glucose cotransporter-2 inhibitor, a vasodilator, a diuretic, a neurohormonal antagonist (e.g., a beta blocker, an angiotensin receptor blocker, such as an angiotensin II receptor blocker, an angiotensin converting enzyme inhibitor, or a mineralocorticoid receptor antagonist), an aldosterone antagonist, a positive inotrope, a soluble guanylate cyclase stimulator, a nitrate derivative, or an angiotensin receptor neprilysin inhibitor (e.g., sacubitril-valsartan).
E42. The method of any one of E39-E41, wherein the subject is administered or is on a single HF background therapy.
E43. The method of any one of E39-E41, wherein the subject is administered or is on two or more HF background therapies.
E44. The method of any one of E39-E43, wherein the HF background therapy is administered or is taken for at least 30 days prior to treatment with a composition of Table 10, Table 11, or Table 12 (e.g., the subject is administered or the subject is on stable HF background therapy for at least 30 days prior to administration of a composition of Table 10, Table 11, or Table 12, stable HF background therapy is defined as no change in dose/regimen of HF background therapy).
E45. The method of any one of E1-E44, wherein the method reduces fibrosis, reduces inflammation, reduces cardiac remodeling, improves cardiac function, or improves ventricular function.
E46. The method of any one of E1-E45, wherein the method slows or inhibits progression of the disease or condition.
E47. The method of any one of E1-E46, wherein the method delays development of or prevents the disease or condition.
E48. The method of any one of E1-E47, wherein the method reduces, delays, or eliminates the need for coronary revascularization, heart valve repair or replacement, implantation of a cardioverter-defibrillator, cardiac resynchronization therapy, a ventricular assist device, or a heart transplant.
E49. The method of any one of E1-E48, wherein the method reduces dyspnea (shortness of breath), orthopnea, paroxysmal nocturnal dyspnea, fatigue, or fluid retention, or increases exercise tolerance.
E50. The method of any one of E1-E49, wherein the method reduces the need for vascular surgery, or reduces secondary cardiovascular events.
E51. The method of any one of E1-E50, wherein the method improves life expectancy.
E52. The method of any one of E1-E51, wherein the method reduces the risk of cardiovascular death.
E53. The method of any one of E1-E52, wherein the method reverses progression of the disease or condition.
E54. The method of any one of E1-E53, wherein the method decreases NT-proBNP levels (e.g., compared to Baseline measurements, for example, by 24 weeks or 32 weeks of treatment with a composition of Table 10, Table 11, or Table 12).
E55. The method of any one of E1-E54, wherein the method improves CPET parameters (e.g., compared to Baseline measurements, for example, by 24 weeks of treatment with a composition of Table 10, Table 11, or Table 12).
E56. The method of any one of E1-E55, wherein the method improves ECHO parameters (e.g., compared to Baseline measurements, for example, by 24 weeks of treatment with a composition of Table 10, Table 11, or Table 12).
E57. The method of any one of E1-E56, wherein the method comprises administering to the subject a therapeutically effective amount of a composition of Table 10.
E58. The method of any one of E1-E57, wherein the composition of Table 10 is a polypeptide comprising an extracellular activin receptor type Ila (ActRIIa) variant, the variant having a sequence of

(SEQ ID NO: 1)
GAILGRSETQECLX₁X₂NANWX₃X₄X₅X₆TNQTGVEX₇CX₈GX₉X₁₀X₁₁X₁₂X₁₃
X₁₄HCX₁₅ATWX₁₆NISGSIEIVX₁₇X₁₈GCX₁₉X₂₀X₂₁DX₂₂NCYDRTDCVEX₂₃
X₂₄X₂₅X₂₆PX₂₇VYFCCCEGNMCNEKFSYFPEMEVTQPTS,

wherein X₁ is F or Y; X₂ is F or Y; X₃ is E or A; X₄ is K or L; X₅ is D or E; X₆ is R or A; X₇ is P or R; X₈ is Y or E; X₉ is D or E; X₁₀ is K or Q; X₁₁ is D or A; X₁₂ is K or A; X₁₃ is R or A; X₁₄ is R or L; X₁₅ is F or Y; X₁₆ is K, R, or A; X₁₇ is K, A, Y, F, or I; X₁₈ is Q or K; X₁₉ is W or A; X₂₀ is L or A; X₂₁ is D, K, R, A, F, G, M, N, or I; X₂₂ is I, F, or A; X₂₃ is K or T; X₂₄ is K or E; X₂₅ is D or E; X₂₆ is S or N; and X₂₇ is E or Q, and
wherein the variant does not have the sequence of any of SEQ ID NOs: 73 and 76-96.
E59. The method of E58, wherein X₁ is F or Y; X₂ is Y; X₄ is L; X₅ is D or E; X₇ is P or R; X₈ is E; X₉ is E; X₁₀ is K or Q; X₁₄ is L; X₁₅ is F or Y; X₁₆ is K or R; X₁₈ is K; X₂₂ is I or F; X₂₃ is T; X₂₄ is K or E; X₂₅ is E; X₂₆ is N; and X₂₇ is Q.
E60. The method of E59, wherein X₁ is F or Y; X₂ is Y; X₃ is E; X₄ is L; X₅ is D or E; X₆ is R; X₇ is P or R; X₈ is E; X₉ is E; X₁₀ is K or Q; X₁₁ is D; X₁₂ is K; X₁₃ is R; X₁₄ is L; X₁₅ is F or Y; X₁₆ is K or R; X₁₇ is K; X₁₈ is K; X₁₉ is W; X₂₀ is L; X₂₁ is D; X₂₂ is I or F; X₂₃ is T; X₂₄ is K or E; X₂₅ is E; X₂₆ is N; and X₂₇ is Q.
E61. The method of E59 or E60, wherein X₁ is F and X₁₀ is K.
E62. The method of E58, wherein the wherein the variant has the sequence of any one of SEQ ID NOs: 6-72.
E63. The method of E63, wherein the variant has the sequence of SEQ ID NO: 69.
E64. The method of any one of E58-E63, wherein the polypeptide further includes an Fc domain monomer fused to the C-terminus of the polypeptide (e.g., the C-terminus of the ActRIIa variant) by way of a linker.
E65. The method of E64, wherein the polypeptide is in the form of a dimer (e.g., a homodimer).
E66. The method of E64, wherein the polypeptide has the sequence of SEQ ID NO: 156.
E67. The method of E66, wherein the polypeptide is in the form of a homodimer.
E68. The method of any one of E1-E56, wherein the method comprises administering to the subject a therapeutically effective amount of a composition of Table 11.
E69. The method of any one of E1-E56 and E68, wherein the composition of Table 11 is a polypeptide comprising an extracellular activin receptor type IIB (ActRIIB) variant, the variant having one or more amino acid substitutions relative to the sequence of GRGEAETRECIYYNANWELERTNQSGLERCEGEQDKRLHCYASWRNSSGTIELVKKGCWLDDF NCYDRQECVATEENPQVYFCCCEGNFCNERFTHLPEAGGPEVTYEPPPTAPT (SEQ ID NO: 45), wherein the variant comprises one or more amino acid substitutions that impart reduced BMP9 binding relative to wild type extracellular ActRIIB and one or more additional amino acid substitutions, wherein the substitutions that reduce BMP9 binding comprise one or more of:
   a) amino acid substitution E75K;
   b) amino acid substitutions Q69T and E70D; or
   c) amino acid substitutions Q69D and E70T,
optionally wherein the variant is truncated from the N-terminus by deletion of 1, 2, 3, 4, 5, 6, or 7 amino acids.
E70. The method of E69, wherein the variant comprises amino acid substitutions I11 L, L27V, Q34K, T50S, I51 L, L53I, Q69D, E70T, E75K, and F89M or amino acid substitutions I11 L, L27V, Q34K, T50S, I51 L, L53I, Q69T, E70D, E75K, and F89M.
E71. The method of E69, wherein the variant has the sequence of any one of SEQ ID NOs: 158-171.
E72. The method of E71, wherein the variant has the sequence of SEQ ID NO: 171.
E73. The method of any one of E69-E72, wherein the polypeptide further includes an Fc domain monomer fused to the C-terminus of the polypeptide (e.g., the C-terminus of the ActRIIB variant) by way of a linker.
E74. The method of E73, wherein the polypeptide is in the form of a dimer (e.g., a homodimer).
E75. The method of E73, wherein the polypeptide has the sequence of SEQ ID NO: 172.
E76. The method of E75, wherein the polypeptide is in the form of a homodimer.
E77. The method of any one of E1-E56, wherein the method comprises administering to the subject a therapeutically effective amount of a composition of Table 12.
E78. The method of any one of E1-E57 and E77, wherein the composition of Table 12 is polypeptide comprising an extracellular ActRII chimera having a sequence of any one of SEQ ID NOs: 174-194, wherein X₁ is D or R, X₂ is I, F, E, D, Y, S, N, Q, or T, X₃ is N or T, X₄ is A or E, X₅ is T or K, X₆ is E or K, X₇ is E or D, X₈ is N or S, and X₉ is Q, E, K, R, D, or N, optionally wherein the chimera is truncated from the N-terminus by deletion of 1, 2, 3, 4, 5, 6, 7, 8, or 9 amino acids, wherein the chimera retains the two amino acids before the first cysteine.
E79. The method of E78, wherein the chimera has the sequence of SEQ ID NO: 192.
E80. The method of E78, wherein the chimera has the sequence of SEQ ID NO: 185.
E81. The method of E78, wherein the chimera has the sequence of SEQ ID NO: 178.
E82. The method of any one of E78-E81, wherein X₁ is D, X₂ is I, F, or E, X₃ is N or T, X₄ is A or E, X₅ is T or K, X₆ is E or K, X₇ is E or D, X₈ is N or S, and X₉ is E or Q.
E83. The method of E82, wherein X₁ is D, X₂ is I or F, X₃ is N, X₄ is A or E, X₅ is T or K, X₆ is E or K, X₇ is E or D, X₈ is N or S, and X₉ is E or Q.
E84. The method of E83, wherein X₁ is D, X₂ is F, X₃ is N, X₄ is E, X₅ is K, X₆ is K, X₇ is D, X₈ is S, and X₉ is Q.
E85. The method of E78, wherein the chimera has the sequence of any one of SEQ ID NOs: 195-216.
E86. The method of E85, wherein the chimera has the sequence of SEQ ID NO: 214.
E87. The method of E85, wherein the chimera has the sequence of SEQ ID NO: 198.
E88. The method of E85, wherein the chimera has the sequence of SEQ ID NO: 213.
E89. The method of any one of E78-E88, wherein the polypeptide further includes an Fc domain monomer fused to the C-terminus of the polypeptide (e.g., the C-terminus of the chimera) by way of a linker.
E90. The method of E89, wherein the polypeptide is in the form of a dimer (e.g., a homodimer).
E91. The method of E89, wherein the polypeptide has the sequence of any one of SEQ ID NOs: 217-220 and SEQ ID NOs: 294-373.
E92. The method of E91, wherein the polypeptide has the sequence of SEQ ID NO: 326.
E93. The method of E91, wherein the polypeptide has the sequence of SEQ ID NO: 220.
E94. The method of E91, wherein the polypeptide has the sequence of SEQ ID NO: 325.
E95. The method of any one of E91-E94, wherein the polypeptide is in the form of a homodimer.
E96. The method of any one of E1-E95, wherein the composition is administered in an amount sufficient to reduce fibrosis, reduce inflammation, reduce cardiac remodeling, improve cardiac function, improve ventricular function, slows or inhibit progression of the disease or condition, delay development of the disease or condition, prevent the disease or condition, reverse progression of the disease or condition, reduce, delay, or eliminate the need for coronary revascularization, heart valve repair or replacement, implantation of a cardioverter-defibrillator, cardiac resynchronization therapy, a ventricular assist device, or a heart transplant, reduce dyspnea, reduce orthopnea, reduce paroxysmal nocturnal dyspnea, reduce fatigue, reduce fluid retention, increase exercise tolerance, improve life expectancy, reduce the risk of cardiovascular death, decrease NT-proBNP levels, improve CPET parameters, or improve ECHO parameters.
E97. The method of any one of E1-E96, wherein the method does not cause a vascular complication in the subject.
E98. The method of E97, wherein the method does not increase vascular permeability or leakage.
E99. The method of any one of E1-E98, wherein the subject is a human.

### Definitions

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the terms “extracellular activin receptor type IIA (ActRIIA) variant” and “ActRIIA variant” refer to a peptide including a soluble, extracellular portion of the single transmembrane receptor, ActRIIA, that has at least one amino acid substitution relative to a wild-type extracellular ActRIIA (e.g., bold portion of the sequence of SEQ ID NO: 75 shown below) or an extracellular ActRIIA having any one of the sequences of SEQ ID NOs: 76-96. The sequence of the wild-type, human ActRIIA precursor protein is shown below (SEQ ID NO: 75), in which the signal peptide is italicized and the extracellular portion is bold.

Wild-type, human ActRIIA precursor protein (SEQ ID NO: 75):
MGAAAKLAFAVFLISCSSGAILGRSETQECLFFNANWEKDRTNQTGVEPCYGDKDKRRHCFAT
WKNISGSIEIVKQGCWLDDINCYDRTDCVEKKDSPEVYFCCCEGNMCNEKFSYFPEMEVTQPT
SNPVTPKPPYYNILLYSLVPLMLIAGIVICAFWVYRHHKMAYPPVLVPTQDPGPPPPSPLLGLKPL
QLLEVKARGRFGCVWKAQLLNEYVAVKIFPIQDKQSWQNEYEVYSLPGMKHENILQFIGAEKRG
TSVDVDLWLITAFHEKGSLSDFLKANVVSWNELCHIAETMARGLAYLHEDIPGLKDGHKPAISHR
DIKSKNVLLKNNLTACIADFGLALKFEAGKSAGDTHGQVGTRRYMAPEVLEGAINFQRDAFLRID
MYAMGLVLWELASRCTAADGPVDEYMLPFEEEIGQHPSLEDMQEVVVHKKKRPVLRDYWQKH
AGMAMLCETIEECWDHDAEARLSAGCVGERITQMQRLTNIITTEDIVTVVTMVTNVDFPPKESSL

An extracellular ActRIIA variant may have a sequence of any one of SEQ ID NOs: 1-72. In particular embodiments, an extracellular ActRIIA variant has a sequence of any one of SEQ ID NOs: 6-72 (Table 2). In some embodiments, an extracellular ActRIIA variant may have at least 85% (e.g., at least 85%, 87%, 90%, 92%, 95%, 97%, or greater) amino acid sequence identity to the sequence of a wild-type extracellular ActRIIA (SEQ ID NO: 73).

As used herein, the terms “extracellular activin receptor type IIB (ActRIIB) variant” and “ActRIIB variant” refer to a peptide including a soluble, extracellular portion of the single transmembrane receptor, ActRIIB, that has at least one amino acid substitution relative to a wild-type extracellular ActRIIB (e.g., bold portion of the sequence of SEQ ID NO: 173 shown below). The sequence of the wild-type, human ActRIIB is shown below (SEQ ID NO: 173), in which the signal peptide is italicized and the extracellular portion is bold.

Wild-type human ActRIIB (SEQ ID NO: 173):
MTAPWVALALLWGSLCAGSGRGEAETRECIYYNANWELERTNQSGLERCEGEQDKRLHCY
ASWRNSSGTIELVKKGCWLDDFNCYDRQECVATEENPQVYFCCCEGNFCNERFTHLPEAG
GPEVTYEPPPTAPTLLTVLAYSLLPIGGLSLIVLLAFWMYRHRKPPYGHVDIHEDPGPPP
PSPLVGLKPLQLLEIKARGRFGCVWKAQLMNDFVAVKIFPLQDKQSWQSEREIFSTPGMK
HENLLQFIAAEKRGSNLEVELWLITAFHDKGSLTDYLKGNIITWNELCHVAETMSRGLSY
LHEDVPWCRGEGHKPSIAHRDFKSKNVLLKSDLTAVLADFGLAVRFEPGKPPGDTHGQVG
TRRYMAPEVLEGAINFQRDAFLRIDMYAMGLVLWELVSRCKAADGPVDEYMLPFEEEIGQ
HPSLEELQEVVVHKKMRPTIKDHWLKHPGLAQLCVTIEECWDHDAEARLSAGCVEERVSL
IRRSVNGTTSDCLVSLVTSVTNVDLPPKESSI

An extracellular ActRIIB variant may have a sequence of any one of SEQ ID NOs: 157-171. In particular embodiments, an extracellular ActRIIB variant has a sequence of any one of SEQ ID NOs: 158-171 (Table 5). In some embodiments, an extracellular ActRIIB variant may have at least 85% (e.g., at least 85%, 87%, 90%, 92%, 95%, 96%, 97%, 98%, 99%, or greater) amino acid sequence identity to the sequence of a wild-type extracellular ActRIIB (SEQ ID NO: 74). The extracellular ActRIIB variant may also have an N-terminal truncation of 1-7 amino acids relative to the extracellular portion of ActRIIB.

As used herein, the terms “extracellular activin receptor type II (ActRII) chimera,” “extracellular ActRII chimera,” and “ActRII chimera” refer to a peptide including a soluble, extracellular portion of the single transmembrane receptor ActRIIB and a soluble, extracellular portion of the single transmembrane receptor ActRIIA. The ActRII chimeras described herein result from joining an N-terminal portion of extracellular ActRIIB to a C-terminal portion of extracellular ActRIIA such that the sequences are contiguous (e.g., the ActRIIA sequence continues where the ActRIIB sequence left off, starting with the next the amino acid located in the corresponding position of ActRIIA). The extracellular ActRII chimera may also include one or more amino acid substitutions in the portion of the chimera that corresponds to the sequence of ActRIIB compared to a wild-type extracellular ActRIIB (e.g., bold portion of the sequence of SEQ ID NO: 173 shown above) and/or one or more amino acid substitutions in the portion of the chimera that corresponds to the sequence of ActRIIA compared to a wild-type extracellular ActRIIA (e.g., bold portion of the sequence of SEQ ID NO: 75 shown above). The extracellular ActRII chimera may also have an N-terminal truncation of 1-9 amino acids relative to the extracellular portion of ActRIIB or ActRIIA. The sequences of wild-type, human ActRIIB (SEQ ID NO: 173) and wild-type, human ActRIIA (SEQ ID NO: 75) are shown in the definitions above, in which the signal peptide is italicized and the extracellular portion is bold. An extracellular ActRII chimera may have the sequence of any one of SEQ ID NOs: 174-216. In particular embodiments, an extracellular ActRII chimera has the sequence of any one of SEQ ID NOs: 195-216 (Table 7).

As used herein, the term “extracellular activin receptor type II (ActRII) variant” refers to an extracellular ActRIIA variant, an extracellular ActRIIB variant, or an extracellular ActRII chimera described herein.

As used herein, the term “N-terminal truncation” refers to a deletion of 1-7 amino acids (e.g., 1, 2, 3, 4, 5, 6, or 7 amino acids) from the N-terminus of an extracellular ActRIIB variant (e.g., an extracellular ActRIIB variant having the sequence of any one of SEQ ID NOs: 157-171 (e.g., SEQ ID NOs: 158-171)) or a deletion of 1-9 amino acids (e.g., 1, 2, 3, 4, 5, 6, 7, 8, or 9 amino acids) from the N-terminus of an extracellular ActRII chimera (e.g., an extracellular ActRII chimera having the sequence of any one of SEQ ID NOs: 174-216 (e.g., SEQ ID NOs: 195-216)). The N-terminal truncation can remove amino acids up to two amino acids before the first cysteine (e.g., the two amino acids before the first cysteine (RE) are retained in an N-terminally truncated extracellular ActRIIB variant and the two amino acids before the first cystine (RE or QE) are retained in an N-terminally truncated extracellular ActRII chimera).

As used herein, the term “linker” refers to a linkage between two elements, e.g., peptides or protein domains. A polypeptide described herein may include an extracellular ActRII variant (e.g., an extracellular ActRIIA variant (e.g., an extracellular ActRIIA variant having a sequence of any one of SEQ ID NOs: 1-72 (e.g., SEQ ID NOs: 6-72)), an extracellular ActRIIB variant (e.g., an extracellular ActRIIB variant having the sequence of any one of SEQ ID NOs: 157-171 (e.g., SEQ ID NOs: 158-171)), or an extracellular ActRII chimera (e.g., an extracellular ActRII chimera having a sequence of any one of SEQ ID NOs: 174-216 (e.g., SEQ ID NOs: 195-216)) fused to an Fc domain monomer. The Fc domain monomer increases stability or improves pharmacokinetic properties of the polypeptide and can be fused to the polypeptide by way of a linker. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 1-200 amino acid sequence) occurring between two elements, e.g., peptides or protein domains, to provide space and/or flexibility between the two elements. An amino acid spacer is part of the primary sequence of a polypeptide (e.g., fused to the spaced peptides via the polypeptide backbone). The formation of disulfide bonds, e.g., between two hinge regions that form an Fc domain, is not considered a linker.

As used herein, the term “Fc domain” refers to a dimer of two Fc domain monomers. An Fc domain has at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the wild-type Fc domain, the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 antibody constant domain to reduce the interaction or binding between the Fc domain and an Fcγ receptor. In some embodiments, the Fc domain contains one or more amino acid substitutions that reduce or inhibit Fc domain dimerization. An Fc domain can be any immunoglobulin antibody isotype, including IgG, IgE, IgM, IgA, or IgD. Additionally, an Fc domain can be an IgG subtype (e.g., IgG1, IgG2a, IgG2b, IgG3, or IgG4). The Fc domain can also be a non-naturally occurring Fc domain, e.g., a recombinant Fc domain.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human red blood cell, platelet, neutrophil, or muscle cell).

As used herein, the term “fused” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage. In some embodiments of a polypeptide described herein, an extracellular ActRII variant (e.g., an extracellular ActRIIA variant (e.g., an extracellular ActRIIA variant having a sequence of any one of SEQ ID NOs: 1-72 (e.g., SEQ ID NOs: 6-72)), an extracellular ActRIIB variant (e.g., an extracellular ActRIIB variant having the sequence of any one of SEQ ID NOs: 157-171 (e.g., SEQ ID NOs: 158-171)), or an extracellular ActRII chimera (e.g., an extracellular ActRII chimera having a sequence of any one of SEQ ID NOs: 174-216 (e.g., SEQ ID NOs: 195-216))) may be fused in tandem series to the N- or C-terminus of an Fc domain monomer (e.g., the sequence of SEQ ID NO: 97, SEQ ID NO: 150, or SEQ ID NO: 155) by way of a linker. For example, an extracellular ActRII variant is fused to an Fc domain monomer by way of a peptide linker, in which the N-terminus of the peptide linker is fused to the C-terminus of the extracellular ActRII variant through a chemical bond, e.g., a peptide bond, and the C-terminus of the peptide linker is fused to the N-terminus of the Fc domain monomer through a chemical bond, e.g., a peptide bond.

As used herein, the term “C-terminal extension” refers to the addition of one or more amino acids to the C-terminus of an extracellular ActRIIA variant (e.g., an extracellular ActRIIA variant having the sequence of any one of SEQ ID NOs: 1-70 (e.g., SEQ ID NOs: 6-70)) or at the C-terminus of a an extracellular ActRII chimera (e.g., an extracellular ActRII chimera having the sequence of any one of SEQ ID NOs: 174-216 (e.g., SEQ ID NOs: 195-216)). The C-terminal extension can be one or more amino acids, such as 1-6 amino acids (e.g., 1, 2, 3, 4, 5, 6 or more amino acids). The C-terminal extension may include amino acids from the corresponding position of wild-type ActRIIA (for an ActRIIA variant) or from the corresponding position of wild-type ActRIIA or ActRIIB (for an ActRII chimera). Exemplary C-terminal extensions are the amino acid sequence NP (a two amino acid C-terminal extension) and the amino acid sequence NPVTPK (SEQ ID NO: 154) (a six amino acid C-terminal extension). Any amino acid sequence that does not disrupt the activity of the polypeptide can be used. SEQ ID NO: 71, which is the sequence of SEQ ID NO: 69 with a C-terminal extension of NP, and SEQ ID NO: 72, which is the sequence of SEQ ID NO: 69 with a C-terminal extension of NPVTPK, represent two of the possible ways that a polypeptide described herein can be modified to include a C-terminal extension.

As used herein, the term “percent (%) identity” refers to the percentage of amino acid (or nucleic acid) residues of a candidate sequence, e.g., an extracellular ActRIIA variant, an extracellular ActRIIB variant, or an extracellular ActRII chimera, that are identical to the amino acid (or nucleic acid) residues of a reference sequence, e.g., a wild-type extracellular ActRIIA (e.g., SEQ ID NO: 73) or wild-type extracellular ActRIIB (e.g., SEQ ID NO: 74), after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis. Serum half-life comparisons can be made by comparing the serum half-life of Fc fusion proteins.

As used herein, the terms “affinity” and “binding affinity” refer to the strength of the binding interaction between two molecules. Generally, binding affinity refers to the strength of the sum total of non-covalent interactions between a molecule and its binding partner, such as an extracellular ActRIIA variant, an extracellular ActRIIB variant, or an extracellular ActRII chimera and BMP9 or activin A. Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (KA). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. The K_{D} of two interacting molecules may be determined using methods and techniques well known in the art, e.g., surface plasmon resonance. K_{D} is calculated as the ratio of k_{off}/kₒₙ.

As used herein, the phrase “affecting myostatin, activin A, activin B, and/or BMP9 signaling” means changing the binding of myostatin, activin A, activin B, and/or BMP9 to their receptors, e.g., ActRIIA, ActRIIB, and/or BMPRII (e.g., ActRIIA or ActRIIB, e.g., endogenous ActRIIA or ActRIIB). In some embodiments, a polypeptide including an extracellular ActRIIA variant, an extracellular ActRIIB variant, or an extracellular ActRII chimera described herein reduces or inhibits the binding of myostatin, activin A, activin B, and/or BMP9 to their receptors, e.g., ActRIIA, ActRIIB, and/or BMPRII (e.g., ActRIIA or ActRIIB, e.g., endogenous ActRIIA or ActRIIB).

As used herein, the terms “increasing” and “decreasing” refer to modulating resulting in, respectively, greater or lesser amounts, of function, expression, or activity of a metric relative to a reference. For example, subsequent to administration of a polypeptide described herein including an extracellular ActRIIA variant, an extracellular ActRIIB variant, or an extracellular ActRII chimera in a method described herein, the amount of a marker of a metric (e.g., inflammation or cardiac remodeling) as described herein may be increased in a subject relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “cardiovascular disease” refers to any disease or disorder of the heart or blood vessels (e.g., arteries and veins) or any symptom thereof, or any disease or condition that causes or contributes to a cardiovascular disease. Non-limiting examples of cardiovascular diseases include arrhythmia, atrial fibrillation, deep vein thrombosis, heart failure, ventricular dysfunction, homozygous familial sitosterolemia, hyperlipidemia, hypertriglyceridemia, mixed dyslipidemia, moderate to mild heart failure, cardiomyopathy, calcification disease, heart valve disease, paroxysmal atrial fibrillation/flutter, primary hyperlipidemia, recurrent hemodynamically unstable ventricular tachycardia (VT), recurrent ventricular arrhythmias, recurrent ventricular fibrillation (VF), aneurysm formation, sitosterolemia, symptomatic atrial fibrillation/flutter, tachycardia, vascular disease, ventricular arrhythmias, and other cardiovascular events.

“Heart failure” refers to a pathological state that is also referred to as myocardial insufficiency or weakness of the heart muscle. Heart failure is characterized by inadequate functioning of the heart, the heart no longer being capable of efficient delivery to comply with the requirements. Heart failure can be categorized according to various aspects. For example, according to the affected segment of the heart it is classified as right heart failure, left heart failure and failure on both sides (global failure). According to the stability of an equilibrium influenced by physiological and therapeutic mechanisms, a distinction is made between compensated and decompensated heart failure. Classification takes place into acute and chronic heart failure according to the time course. Causes of heart failure are, inter alia, myocardial infarction, cardiomyopathy, inborn or acquired cardiac defects, essential or pulmonary hypertension, cardiac arrhythmias, coronary heart disease, or myocarditis.

As used herein, the term “vascular complication” refers to a vascular disorder or any damage to the blood vessels, such as damage to the blood vessel walls. Damage to the blood vessel walls may cause an increase in vascular permeability or leakage. The term “vascular permeability or leakage” refers to the capacity of the blood vessel walls to allow the flow of small molecules, proteins, and cells in and out of blood vessels. An increase in vascular permeability or leakage may be caused by an increase in the gaps (e.g., an increase in the size and/or number of the gaps) between endothelial cells that line the blood vessel walls and/or thinning of the blood vessel walls.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including an extracellular ActRIIA variant, an extracellular ActRIIB variant, or an extracellular ActRII chimera fused to an Fc domain monomer may form a homodimer through the interaction of two Fc domain monomers, which form an Fc domain in the homodimer.

As used herein, the term “heterodimer” refers to a molecular construct formed by two different macromolecules, such as proteins or nucleic acids. The two monomers may form a heterodimer by covalent bonds or non-covalent bonds. For example, a polypeptide described herein including an extracellular ActRIIA variant, an extracellular ActRIIB variant, or an extracellular ActRII chimera fused to an Fc domain monomer may form a heterodimer through the interaction of two Fc domain monomers, each fused to a different extracellular ActRIIA variant, extracellular ActRIIB variant, or extracellular ActRII chimera, respectively, which form an Fc domain in the heterodimer (e.g., a heterodimer can contain two different extracellular ActRIIA variants, two different extracellular ActRIIB variants, or two different extracellular ActRII chimeras).

As used herein, the term “low transfusion burden” refers to a condition of a subject that has received less than four units of red blood cells (RBCs) within eight weeks (e.g., 3, 2, 1, or 0 units of RBCs within eight weeks) prior to treatment with an ActRII variant described herein. A subject with a low transfusion burden can be identified as having anemia based on measurements of mean hemoglobin concentration. A subject with a low transfusion burden and a mean hemoglobin concentration of less than 10.0 g/dL of two measurements performed at least one week apart prior to treatment with an ActRII variant described herein (e.g., one measurement performed within one day prior to treatment and the other performed 7-28 days prior, not influenced by RBC transfusion within seven days of measurement) is defined as having anemia. In some embodiments, a subject with a low transfusion burden receives 1-3 units of RBCs (1-3 RBC transfusions) within eight weeks prior to treatment with an ActRII variant described herein. In some embodiments, a subject with a low transfusion burden does not receive any units of RBCs (0 RBC transfusions) within eight weeks prior to treatment with an ActRII variant described herein. A subject with a low transfusion burden who does not receive any units of RBCs (0 RBC transfusions) within eight weeks prior to treatment with an ActRII variant described herein can also be referred to as a “non-transfused” subject.

As used herein, the term “high transfusion burden” refers to a condition of a subject requiring greater than or equal to four units of RBCs (e.g., 4, 5, 6, 7, 8, or more units) within eight weeks prior to treatment with an ActRII variant described herein. A subject with a high transfusion burden can be identified as having anemia based on measurements of mean hemoglobin concentration. A subject with a high transfusion burden and a mean hemoglobin concentration of less than or equal to 9.0 g/dL is defined as having anemia.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector of the invention or a pharmaceutical composition containing a polypeptide, nucleic acid, or vector of the invention effective in achieving the desired therapeutic effect in treating a patient having or at risk of developing a disease or condition, such as a cardiovascular disease (e.g., cardiomyopathy, a calcification disease, heart valve disease, vasculitis, deep vein thrombosis, left-sided heart failure (e.g., heart failure with reduced ejection fraction (HFrEF) or heart failure with preserved ejection fraction (HFpEF), such as chronic HFrEF or chronic HFpEF), right-sided heart failure, an arrhythmia, ventricular dysfunction, an aneurysm, backward heart failure, forward heart failure, high-output heart failure, low-output heart failure, compensated heart failure, or decompensated heart failure). In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition of the present invention includes pharmaceutically acceptable components that are compatible with the polypeptide, nucleic acid, or vector. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to the polypeptide including an extracellular ActRIIA variant, an extracellular ActRIIB variant, or an extracellular ActRII chimera, the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, the term “treating and/or preventing” refers to the treatment and/or prevention of a disease or condition, such as a cardiovascular disease (e.g., cardiomyopathy, a calcification disease, heart valve disease, vasculitis, deep vein thrombosis, left-sided heart failure (e.g., heart failure with reduced ejection fraction (HFrEF) or heart failure with preserved ejection fraction (HFpEF), such as chronic HFrEF or chronic HFpEF), right-sided heart failure, backward heart failure, forward heart failure, high-output heart failure, low-output heart failure, compensated heart failure, decompensated heart failure, an arrhythmia, ventricular dysfunction, or an aneurysm) using methods and compositions of the invention. Generally, treating a cardiovascular disease occurs after a subject has developed the disease. Preventing a cardiovascular disease refers to steps or procedures taken when a subject is at risk of developing the disease. The subject may show signs or mild symptoms that are judged by a physician to be indications or risk factors for developing a cardiovascular disease, have another disease or condition associated with the development of a cardiovascular disease, be undergoing treatment that may cause a cardiovascular disease, or have a family history or genetic predisposition to developing a cardiovascular disease, but has not yet developed the disease.

As used herein, the term “subject” refers to a mammal, e.g., preferably a human. Mammals include, but are not limited to, humans and domestic and farm animals, such as monkeys (e.g., a cynomolgus monkey), mice, dogs, cats, horses, and cows, etc.

### DESCRIPTION OF THE DRAWINGS

FIG. 1 is a heat map showing changes in serum proteins in healthy postmenopausal women administered a single dose of ActRIIB 2.12-Fc at 4.5 mg/kg. Administration of ActRIIB 2.12-Fc altered serum proteins associated with inflammation and extracellular matrix remodeling pathways.

FIGS. 2A-2J are a series of graphs showing representative examples of serum proteins altered by ActRIIB 2.12-Fc administration in post-menopausal women. ActRIIB 2.12-Fc decreased serum collagen fragments (FIGS. 2A-2B), fibrosis markers (FIGS. 2C-2D), and pro-inflammatory cytokines (FIGS. 2E-2F) and increased anti-inflammatory cytokines (FIGS. 2G-2H) and markers of macrophage repolarization (FIGS. 2I-2J). Data are represented as mean fold change from baseline ±SEM, N=5-6 per group. COL2A1=Collagen type II; COL3A1=Collagen Type III; IL=Interleukin; MMP-7=matrix metalloproteinase-7; MMP10=matrix metalloproteinase-10; MARCO=Macrophage Receptor with Collagenous Structure; sCD163=soluble cluster of differentiation 163.

FIGS. 3A-3B are a series of graphs showing that ActRIIB 2.12-Fc reduced serum NT pro-BNP as assessed by ELISA and SOMASCAN® in healthy post-menopausal women, a population known to have an elevated risk of cardiovascular disease. ELISA data are shown in FIG. 3A and SOMASCAN® data are shown in FIG. 3B.

FIGS. 4A-4C are a series of graphs showing that ActRIIB 2.12-mFc (a homodimer of SEQ ID NO: 171 linked to a mouse Fc domain monomer by a GGG linker) preventative and therapeutic treatment reduced cardiac remodeling. Vehicle-treated mice had increased heart weight (FIG. 4A), left ventricular posterior wall thickness (FIG. 4B), and interventricular septal end diastole (FIG. 4C) compared to Sham animals. ActRIIB 2.12-mFc Preventative mice had significantly reduced heart weight (FIG. 4A), left ventricular posterior wall thickness end diastole (FIG. 4B), and interventricular septal end diastole (FIG. 4C) compared to TAC-vehicle mice. ActRIIB 2.12-mFc Treatment significantly reduced left ventricular posterior wall thickness (FIG. 4B) and interventricular septal end diastole (FIG. 4C) compared to TAC-Vehicle mice. Heart weight in ActRIIB 2.12-mFc Treatment trended towards a decrease compared to TAC-vehicle mice (FIG. 4A). Data represented as mean±SEM. Data analyzed using one-way ANOVA followed by Dunnett post hoc test. *p<0.05; **p<0.01; ***p<0.001; ****p<0.0001; ns—not significant.

FIGS. 5A-5C are a series of graphs and images showing that ActRIIB 2.12-mFc improved left ventricular function. Vehicle treated TAC mice had significantly reduced E/A ratio (FIG. 5A) and increased left ventricular filling pressure compared to Sham mice (FIG. 5B). ActRIIB 2.12-mFc Preventative mice had trends toward increase and decrease in E/A and E/E′ ratio, respectively (FIGS. 5A-5B) versus TAC-vehicle mice. ActRIIB 2.12-mFc Treatment mice had significantly decreased left ventricular filling pressure (FIG. 5B) compared to TAC-vehicle. No significant difference was observed in E/A ratio (FIG. 5A). Representative parasternal long axis view of left ventricle (FIG. 5C). Data represented as mean±SEM. Data analyzed using one-way ANOVA followed by Dunnett post hoc test. *p≤0.05; **p≤0.01; ***p≤0.001; ****p≤0.0001; ns—not significant.

FIGS. 6A-6B are a graph and series of images showing that ActRIIB 2.12-mFc reduced cardiac fibrosis in the left ventricle. Vehicle treated mice had significantly increased left ventricular fibrosis (FIG. 6A) compared to Sham mice. ActRIIB 2.12-mFc Preventative mice had significantly reduced left ventricular fibrosis compared to TAC-Vehicle. Fibrosis in ActRIIB 2.12-mFc Treatment mice trended toward a reduction compared to TAC-Vehicle (FIG. 6A). Representative lung histological sections stained with Picrosirius Red (FIG. 6B). Data represented as mean±SEM. Data analyzed using one-way ANOVA followed by Dunnett post hoc test. *p≤0.05; **p≤0.01; ***p≤0.001; ****p≤0.0001; ns—not significant.

FIG. 7 is a graph showing that ActRIIB 2.12-mFc reduced markers of tissue remodeling in the left ventricle. Vehicle treated TAC mice had significantly increased left ventricle (LV) expression of secreted phosphoprotein 1 (Spp1), TIMP metallopeptidase inhibitor 1 (Timp1), and collagen type I alpha chain 1 (Col1a1) compared to Sham mice. No significant change in expression was observed in transforming growth factor-β (TGF-β). ActRIIB 2.12-mFc Preventative mice had significantly decreased expression of Spp1, Timp1, and Col1a1 versus TAC-vehicle mice. No significant changes were observed in TGF-β. ActRIIB 2.12-mFc Treatment mice had significantly reduced expression of Timp1 and Col1a1 relative to TAC-vehicle. No significant changes were observed in Spp1 or TGF-β. Data represented as mean±SEM. Data analyzed using one-way ANOVA followed by Dunnett post hoc test. *p≤0.05; **p≤0.01; ***p≤0.001; ****p≤0.0001; ns—not significant.

FIGS. 8A-8E are a series of graphs showing that ActRIIa/b-mFc (a homodimer of SEQ ID NO: 69 linked to a mouse Fc domain monomer by a GGG linker) attenuated cardiac remodeling in a transverse aortic constriction model of heart failure. Ejection fraction (FIG. 8A), fractional shortening (FIG. 8B), left ventricle mass (FIG. 8C), left ventricle volume during diastole (FIG. 8D), and left ventricle volume during systole (FIG. 8E) were evaluated. All data represented as mean±SEM. Data analyzed using a student's T Test. *p≤0.05; **p≤0.01; ***p≤0.001; ****p≤0.0001; ns—not significant. Percent change compared to TAC-Vehicle.

FIGS. 9A-9C are a series of graphs showing that ActRIIa/b-hFc (a homodimer of SEQ ID NO: 156) decreased NT-proBNP levels in participants with a lower-risk myelodysplastic syndrome (LR-MDS) and elevated levels of NT-proBNP at baseline. Six MDS participants were identified to have elevated (2450 pg/mL) levels of NT-proBNP at baseline (FIG. 9A). Most had a high transfusion burden (HTB), but the highest baseline levels were observed among two participants with low transfusion burden (LTB). Among the six participants with elevated NT-proBNP levels at baseline, decreases were observed with treatment with ActRIIa/b-hFc in both hematological responders and non-responders (FIG. 9B, each line represents data from a single participant). A transfusion-dependent (LTB) participant (FIG. 9C, 81-year-old female with MDS-RS-MLD) was observed to experience improvements in several biomarkers relevant to MDS, including hemoglobin (Hgb), soluble transferrin receptor (sTfR), ferritin, platelet counts, and NT-proBNP.

FIGS. 10A-10B are a series of graphs showing that ActRIIa/b-hFc decreased NT-proBNP levels in participants with a LR-MDS and elevated levels of NT-proBNP at baseline. Levels of NT-proBNP at baseline are shown in FIG. 10A. FIG. 10B shows changes in NT-proBNP levels in eight trial participants with LR-MDS who had a baseline NT-proBNP level of >450 pg/mL.

### DETAILED DESCRIPTION OF THE INVENTION

The invention features methods of treating cardiovascular disease or reducing cardiovascular risk by administering to a subject a polypeptide including an extracellular activin receptor type II (ActRII) variant, such as an extracellular ActRIIA variant, an extracellular ActRIIB variant, or an extracellular ActRII chimera. In some embodiments, a polypeptide described herein includes an extracellular ActRII variant fused to an Fc domain monomer. A polypeptide including an extracellular ActRII variant fused to an Fc domain monomer may also form a dimer (e.g., homodimer or heterodimer) through the interaction between two Fc domain monomers. The ActRII variants described herein may bind to activins (e.g., activin A and/or activin B), myostatin, and GDF-11, and may have weak binding affinity or no binding affinity to bone morphogenetic protein 9 (BMP9). Treatment of a cardiovascular disease with a polypeptide including an extracellular ActRII variant described herein may reduce fibrosis, reduce inflammation (e.g., vascular inflammation or cardiac inflammation), reduce cardiac remodeling, improve cardiac function, improve ventricular function, slow or inhibit progression of the cardiovascular disease, delay the development of the cardiovascular disease, or prevent the cardiovascular disease. A polypeptide including an ActRII variant described herein may also reduce cardiovascular risk (e.g., reduce the risk of developing a cardiovascular disease, reduce the risk or occurrence of cardiovascular events, reduce cardiac strain, improve cardiac function, or reduce the risk of death associated with a cardiovascular disease) in a subject, such as a subject being treated with a polypeptide including an ActRII variant for a disease or condition other than a cardiovascular disease (e.g., MDS).

Extracellular activin receptor type II variants Activin type II receptors are single transmembrane domain receptors that modulate signals for ligands in the transforming growth factor β (TGF-β) superfamily. Ligands in the TGF-β superfamily are involved in a host of physiological processes, such as muscle growth, vascular growth, cell differentiation, homeostasis, hematopoiesis, and osteogenesis. Examples of ligands in the TGF-β superfamily include, e.g., activin A, activin B, inhibin, growth differentiation factors (GDFs) (e.g., GDF8, also known as myostatin, and GDF11), and bone morphogenetic proteins (BMPs) (e.g., BMP9).

There exist two types of activin type II receptors: ActRIIA and ActRIIB. Studies have shown that BMP9 binds ActRIIB with about 300-fold higher binding affinity than ActRIIA (see, e.g., Townson et al., J. Biol. Chem. 287:27313, 2012). ActRIIA-Fc is known to have a longer half-life compared to ActRIIB-Fc. Described herein are three types of ActRII variants: 1) extracellular ActRIIA variants that are constructed by introducing amino acid residues of ActRIIB into ActRIIA, with the goal of imparting physiological properties conferred by ActRIIB, while also maintaining beneficial physiological and pharmacokinetic properties of ActRIIA; 2) extracellular ActRIIB variants that are constructed by introducing amino acid residues of ActRIIA into ActRIIB, or by introducing novel amino acid substitutions, with the goal of reducing BMP9 binding to prevent or reduce disruption of endogenous BMP9 signaling; and 3) extracellular ActRII chimeras that are constructed by combining portions of extracellular ActRIIA and ActRIIB with the goal of generating proteins that bind to ActRII ligands (e.g., activin A, activin B, myostatin, and GDF11) and retain the function of wild-type extracellular ActRII proteins.

The present invention is based, in part, on the discovery that administration of a polypeptide including an ActRIIB variant described herein (an ActRIIB variant-Fc polypeptide) to healthy human subjects decreased serum proteins involved in inflammation and fibrosis and increased anti-inflammatory cytokines. Human subjects administered the ActRIIB variant also showed decreases in serum NT pro-BNP, a biomarker of cardiac dysfunction. In addition, administration of an ActRIIB variant-Fc polypeptide to a mouse model of transverse aortic constriction (TAC) was found to lead to attenuation of cardiac remodeling and fibrosis when administered either preventatively or as a treatment two weeks after TAC surgery. Administration of an ActRIIA variant-Fc polypeptide to a mouse model of TAC was also found to attenuate cardiac remodeling when administered four weeks after TAC surgery, and administration of an ActRIIA variant-Fc polypeptide to human subjects with lower-risk MDS, anemia, and elevated NT-proBNP levels (≥2450 pg/mL) was found to decrease NT-proBNP levels in both hematological responders and non-responders. Failure to resolve inflammation and altered immune responses underlie the pathology of cardiovascular disease. Accordingly, these data suggest that polypeptides including an ActRII variant, such as an ActRIIB variant or an ActRIIA variant described herein, could be used be used to treat cardiovascular disease or to prevent, delay, or reduce the likelihood of developing cardiovascular disease.

### ActRIIA Variants

In some embodiments, the ActRII variant for use according to the methods described herein is an extracellular ActRIIA variant constructed by introducing amino acid residues of ActRIIB to ActRIIA, with the goal of imparting physiological properties conferred by ActRIIB, while also maintaining beneficial physiological and pharmacokinetic properties of ActRIIA. The preferred ActRIIA variants also exhibit similar or improved binding to activins and/or myostatin compared to wild-type ActRIIA, which allows them to compete with endogenous activin receptors for ligand binding and reduce or inhibit endogenous activin receptor signaling. In some embodiments, amino acid substitutions may be introduced to an extracellular ActRIIA variant to reduce or remove the binding affinity of the variant to BMP9. The wild-type amino acid sequences of the extracellular portions of human ActRIIA and ActRIIB are shown below.

Human ActRIIA, extracellular portion (SEQ ID NO: 73):
GAILGRSETQECLFFNANWEKDRTNQTGVEPCYGDKDKRRHCFATWKNISGSIEIVKQGC
WLDDINCYDRTDCVEKKDSPEVYFCCCEGNMCNEKFSYFPEMEVTQPTS
Human ActRIIB, extracellular portion (SEQ ID NO: 74):
GRGEAETRECIYYNANWELERTNQSGLERCEGEQDKRLHCYASWRNSSGTIELVKKGCWL
DDFNCYDRQECVATEENPQVYFCCCEGNFCNERFTHLPEAGGPEVTYEPPPTAPT

Polypeptides described herein include an extracellular ActRIIA variant having at least one amino acid substitution relative to the wild-type extracellular ActRIIA having the sequence of SEQ ID NO: 73 or the extracellular ActRIIA having any one of the sequences of SEQ ID NOs: 76-96. Possible amino acid substitutions at 27 different positions may be introduced to an extracellular ActRIIA variant (Table 1). In some embodiments, an extracellular ActRIIA variant may have at least 85% (e.g., at least 85%, 87%, 90%, 92%, 95%, 97%, or greater) amino acid sequence identity to the sequence of a wild-type extracellular ActRIIA (SEQ ID NO: 73). An extracellular ActRIIA variant may have one or more (e.g., 1-27, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a wild-type extracellular ActRIIA (SEQ ID NO: 73). In some embodiments, an extracellular ActRIIA variant (e.g., an extracellular ActRIIA variant having a sequence of SEQ ID NO: 1) may include amino acid substitutions at all of the 27 positions as listed in Table 1. In some embodiments, an extracellular ActRIIA variant may include amino acid substitutions at a number of positions, e.g., at 2, 4, 6, 8, 10, 12, 14, 16, 18, 20, 22, 24, or 26 out of the 27 positions, as listed in Table 1.

Amino acid substitutions can worsen or improve the activity and/or binding affinity of the ActRIIA variants described herein. To maintain polypeptide function, it is important that the lysine (K) at position X₁₇ in the sequences shown in Tables 1 and 2 (SEQ ID NOs: 1-72 (e.g., SEQ ID NOs: 6-72)) be retained. Substitutions at that position can lead to a loss of activity. For example, an ActRIIA variant having the sequence GAILGRSETQECLFYNANWELERTNQTGVERCEGEKDKRLHCYATWRNISGSIEIVAKGCWLDDFNCYD RTDCVETEENPQVYFCCCEGNMCNEKFSYFPEMEVTQPTS (SEQ ID NO: 149) has reduced activity in vivo, indicating that the substitution of alanine (A) for lysine (K) at X₁₇ is not tolerated. ActRIIA variants described herein, including variants in Tables 1 and 2 (e.g., SEQ ID NOs: 1-72 (e.g., SEQ ID NOs: 6-72), therefore, retain amino acid K at position X₁₇.

The ActRIIA variants described herein preferably have reduced, weak, or no substantial binding to BMP9. BMP9 binding is reduced in ActRIIA variants (e.g., reduced compared to wild-type ActRIIA) containing the amino acid sequence TEEN (SEQ ID NO: 374) at positions X₂₃, X₂₄, X₂₅, and X₂₆, as well as in variants that maintain the amino acid K at position X₂₄ and have the amino acid sequence TKEN (SEQ ID NO: 375) at positions X₂₃, X₂₄, X₂₅, and X₂₆. The sequences TEEN (SEQ ID NO: 374) and TKEN (SEQ ID NO: 375) can be employed interchangeably in the ActRIIA variants (e.g., the variants in Tables 1 and 2, e.g., SEQ ID NOs: 1-72 (e.g., SEQ ID NOs: 6-72)) described herein to provide reduced BMP9 binding.

The ActRIIA variants described herein may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the variants shown in Tables 1 and 2 (e.g., SEQ ID NOs: 1-70 (e.g., SEQ ID NOs: 6-70)). The C-terminal extension may correspond to sequence from the same position in wild-type ActRIIA. One potential C-terminal extension that can be included in the ActRIIA variants described herein is amino acid sequence NP. For example, a sequence including the C-terminal extension NP is SEQ ID NO: 71 (e.g., SEQ ID NO: 69 with a C-terminal extension of NP). Another exemplary C-terminal extension that can be included in the ActRIIA variants described herein is amino acid sequence NPVTPK (SEQ ID NO: 154). For example, a sequence including the C-terminal extension NPVTPK is SEQ ID NO: 72 (e.g., SEQ ID NO: 69 with a C-terminal extension of NPVTPK).

TABLE 1
Amino acid substitutions in an extracellular ActRIIA variant having
a sequence of any one of SEQ ID NOs: 1-5

GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NISGSIEIV
X₁₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS
(SEQ ID NO: 1)
GAILGRSETQECLFX₂NANWX₃X₄X₅X₆TNQTGVEX₇CX₈GX₉KX₁₁X₁₂X₁₃X₁₄HCX₁₅ATWX₁₆NISGSIEIV
X₁₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS
(SEQ ID NO: 2)
GAILGRSETQECLFX₂NANWEX₄X₅RTNQTGVEX₇CX₈GX₉KDKRX₁₄HCX₁₅ATWX₁₆NISGSIEIVKX₁₈GC
WLDDX₂₂NCYDRTDCVEX₂₃X₂₄X₂₅X₂₆PX₂₇VYFCCCEGNMCNEKFSYFPEMEVTQPTS (SEQ ID NO: 3)
GAILGRSETQECLFX₂NANWEX₄DRTNQTGVEX₇CX₈GX₉KDKRX₁₄HCX₁₅ATWX₁₆NISGSIEIVKX₁₈GC
WLDDX₂₂NCYDRTDCVEX₂₃KX₂₅X₂₆PX₂₇VYFCCCEGNMCNEKFSYFPEMEVTQPTS (SEQ ID NO: 4)
GAILGRSETQECLFX₂NANWEX₄DRTNQTGVEPCX₈GX₉KDKRX₁₄HCFATWKNISGSIEIVKX₁₈GCWLD
DINCYDRTDCVEX₂₃KX₂₅X₂₆PX₂₇VYFCCCEGNMCNEKFSYFPEMEVTQPTS (SEQ ID NO: 5)

| | | | |
|---|---|---|---|
| X₁ | F or Y | X₁₅ | For Y |
| X₂ | F or Y | X₁₆ | K, R, or A |
| X₃ | E or A | X₁₇ | K, A, Y, F, or l |
| X₄ | K or L | X₁₈ | Q or K |
| X₅ | D or E | X₁₉ | W or A |
| X₆ | R or A | X₂₀ | L or A |
| X₇ | P or R | X₂₁ | D, K, R, A, F, G, M, N, |
| | | | or I |
| X₈ | Y or E | X₂₂ | I, F, or A |
| X₉ | D or E | X₂₃ | Kor T |
| X₁₀ | K or Q | X₂₄ | Kor E |
| X₁₁ | D or A | X₂₅ | D or E |
| X₁₂ | K or A | X₂₆ | S or N |
| X₁₃ | R or A | X₂₇ | E or Q |

GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NISGSIEIV
X₁₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS
(SEQ ID NO: 1)
GAILGRSETQECLFX₂NANWX₃X₄X₅X₆TNQTGVEX₇CX₈GX₉KX₁₁X₁₂X₁₃X₁₄HCX₁₅ATWX₁₆NISGSIEIVX₁₇
X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS
(SEQ ID NO: 2)
GAILGRSETQECLFX₂NANWEX₄X₅RTNQTGVEX₇CX₈GX₉KDKRX₁₄HCX₁₅ATWX₁₆NISGSIEIVKX₁₈GC
WLDDX₂₂NCYDRTDCVEX₂₃X₂₄X₂₅X₂₆PX₂₇VYFCCCEGNMCNEKFSYFPEMEVTQPTS (SEQ ID NO: 3)
GAILGRSETQECLFX₂NANWEX₄DRTNQTGVEX₇CX₈GX₉KDKRX₁₄HCX₁₅ATWX₁₆NISGSIEIVKX₁₈GC
WLDDX₂₂NCYDRTDCVEX₂₃KX₂₅X₂₆PX₂₇VYFCCCEGNMCNEKFSYFPEMEVTQPTS (SEQ ID NO: 4)
GAILGRSETQECLFX₂NANWEX₄DRTNQTGVEPCX₈GX₉KDKRX₁₄HCFATWKNISGSIEIVKX₁₈GCWLD
DINCYDRTDCVEX₂₃KX₂₅X₂₆PX₂₇VYFCCCEGNMCNEKFSYFPEMEVTQPTS (SEQ ID NO: 5)

| | |
|---|---|
| X₁₄ | R or L |

In some embodiments of the extracellular ActRIIA variant having the sequence of SEQ ID NO: 1 or 2, X₃ is E, X₆ is R, X₁₁ is D, X₁₂ is K, X₁₃ is R, X₁₆ is K or R, X₁₇ is K, X₁₉ is W, X₂₀ is L, X₂₁ is D, and X₂₂ is I or F. In some embodiments of the extracellular ActRIIA variant having the sequence of SEQ ID NO: 1, X₂ is Y; X₄ is L; X₈ is E; X₉ is E; X₁₄ is L; X₁₈ is K; X₂₃ is T; X₂₅ is E; X₂₆ is N; and X₂₇ is Q. These substitutions in SEQ ID NO: 1 can also be made in SEQ ID NOs: 2-5. In some embodiments of the extracellular ActRIIA variant having the sequence of SEQ ID NO: 1, X₁ is F or Y; X₂ is Y; X₄ is L; X₅ is D or E; X₇ is P or R; X₈ is E; X₉ is E; X₁₀ is K or Q; K₁₄ is L; X₁₅ is F or Y; X₁₆ is K or R; X₁₈ is K; X₂₂ is I or F; X₂₃ is T; X₂₄ is K or E; X₂₅ is E; X₂₆ is N; and X₂₇ is Q. In some embodiments of the extracellular ActRIIA variant having the sequence of SEQ ID NO: 1, X₁ is F or Y; X₂ is Y; X₃ is E; X₄ is L; X₅ is D or E; X₆ is R; X₇ is P or R; X₈ is E; X₉ is E; X₁₀ is K or Q; X₁₁ is D; X₁₂ is K; X₁₃ is R; X₁₄ is L; X₁₅ is F or Y; X₁₆ is K or R; X₁₇ is K; X₁₈ is K; X₁₉ is W; X₂₀ is L; X₂₁ is D; X₂₂ is I or F; X₂₃ is T; X₂₄ is K or E; X₂₅ is E; X₂₆ is N; and X₂₇ is Q. In some embodiments of the extracellular ActRIIA variant having the sequence of SEQ ID NO: 1 or 2, X₁₇ is K. In some embodiments of the extracellular ActRIIA variant having the sequence of SEQ ID NOs: 1-3, X₁₇ is K, X₂₃ is T, X₂₄ is E, X₂₅ is E, and X₂₆ is N. In some embodiments of the extracellular ActRIIA variant having the sequence of any one of SEQ ID NOs: 1-5, X₁₇ is K, X₂₃ is T, X₂₄ is K, X₂₅ is E, and X₂₆ is N.

In some embodiments, a polypeptide described herein includes an extracellular ActRIIA variant having a sequence of any one of SEQ ID NOs: 6-72 (Table 2).

TABLE 2
Extracellular ActRIIA variants having the sequences
of SEQ ID NOs: 6-72

| SEQ | |
|---|---|
| ID NO: | Amino Acid Sequence |
| 6 | GAILGRSETQECLFYNANWELDRTNQTGVEPCEGEKDKRLHCFATWKNISGSIEIV |
| | KKGCWLDDINCYDRTDCVETKENPQVYFCCCEGNMCNEKFSYFPEMEVTQPTS |
| 7 | GAILGRSETQECLFYNANWELERTNQTGVEPCEGEKDKRLHCFATWKNISGSIEIV |
| | KKGCWLDDINCYDRTDCVETKENPQVYFCCCEGNMCNEKFSYFPEMEVTQPTS |
| 8 | GAILGRSETQECLFYNANWELDRTNQTGVERCEGEKDKRLHCFATWKNISGSIEIV |
| | KKGCWLDDINCYDRTDCVETKENPQVYFCCCEGNMCNEKFSYFPEMEVTQPTS |
| 9 | GAILGRSETQECLFYNANWELDRTNQTGVEPCEGEKDKRLHCYATWKNISGSIEIV |
| | KKGCWLDDINCYDRTDCVETKENPQVYFCCCEGNMCNEKFSYFPEMEVTQPTS |
| 10 | GAILGRSETQECLFYNANWELDRTNQTGVEPCEGEKDKRLHCFATWRNISGSIEIV |
| | KKGCWLDDINCYDRTDCVETKENPQVYFCCCEGNMCNEKFSYFPEMEVTQPTS |
| 11 | GAILGRSETQECLFYNANWELDRTNQTGVEPCEGEKDKRLHCFATWKNISGSIEIV |
| | KKGCWLDDFNCYDRTDCVETKENPQVYFCCCEGNMCNEKFSYFPEMEVTQPTS |
| 12 | GAILGRSETQECLFYNANWELDRTNQTGVEPCEGEKDKRLHCFATWKNISGSIEIV |
| | KKGCWLDDINCYDRTDCVETEENPQVYFCCCEGNMCNEKFSYFPEMEVTQPTS |
| 13 | GAILGRSETQECLFYNANWELERTNQTGVERCEGEKDKRLHCFATWKNISGSIEIV |
| | KKGCWLDDINCYDRTDCVETKENPQVYFCCCEGNMCNEKFSYFPEMEVTQPTS |
| 14 | GAILGRSETQECLFYNANWELERTNQTGVEPCEGEKDKRLHCYATWKNISGSIEIV |
| | KKGCWLDDINCYDRTDCVETKENPQVYFCCCEGNMCNEKFSYFPEMEVTQPTS |
| 15 | GAILGRSETQECLFYNANWELERTNQTGVEPCEGEKDKRLHCFATWRNISGSIEIV |
| | KKGCWLDDINCYDRTDCVETKENPQVYFCCCEGNMCNEKFSYFPEMEVTQPTS |
| 16 | GAILGRSETQECLFYNANWELERTNQTGVEPCEGEKDKRLHCFATWKNISGSIEIV |
| | KKGCWLDDFNCYDRTDCVETKENPQVYFCCCEGNMCNEKFSYFPEMEVTQPTS |
| 17 | GAILGRSETQECLFYNANWELERTNQTGVEPCEGEKDKRLHCFATWKNISGSIEIV |
| | KKGCWLDDINCYDRTDCVETEENPQVYFCCCEGNMCNEKFSYFPEMEVTQPTS |
| 18 | GAILGRSETQECLFYNANWELDRTNQTGVERCEGEKDKRLHCYATWKNISGSIEIV |
| | KKGCWLDDINCYDRTDCVETKENPQVYFCCCEGNMCNEKFSYFPEMEVTQPTS |
| 19 | GAILGRSETQECLFYNANWELDRTNQTGVERCEGEKDKRLHCFATWRNISGSIEIV |
| | KKGCWLDDINCYDRTDCVETKENPQVYFCCCEGNMCNEKFSYFPEMEVTQPTS |
| 20 | GAILGRSETQECLFYNANWELDRTNQTGVERCEGEKDKRLHCFATWKNISGSIEIV |
| | KKGCWLDDFNCYDRTDCVETKENPQVYFCCCEGNMCNEKFSYFPEMEVTQPTS |
| 21 | GAILGRSETQECLFYNANWELDRTNQTGVERCEGEKDKRLHCFATWKNISGSIEIV |
| | KKGCWLDDINCYDRTDCVETEENPQVYFCCCEGNMCNEKFSYFPEMEVTQPTS |
| 22 | GAILGRSETQECLFYNANWELDRTNQTGVEPCEGEKDKRLHCYATWRNISGSIEIV |
| | KKGCWLDDINCYDRTDCVETKENPQVYFCCCEGNMCNEKFSYFPEMEVTQPTS |
| 23 | GAILGRSETQECLFYNANWELDRTNQTGVEPCEGEKDKRLHCYATWKNISGSIEIV |
| | KKGCWLDDFNCYDRTDCVETKENPQVYFCCCEGNMCNEKFSYFPEMEVTQPTS |
| 24 | GAILGRSETQECLFYNANWELDRTNQTGVEPCEGEKDKRLHCYATWKNISGSIEIV |
| | KKGCWLDDINCYDRTDCVETEENPQVYFCCCEGNMCNEKFSYFPEMEVTQPTS |
| 25 | GAILGRSETQECLFYNANWELDRTNQTGVEPCEGEKDKRLHCFATWRNISGSIEIV |
| | KKGCWLDDFNCYDRTDCVETKENPQVYFCCCEGNMCNEKFSYFPEMEVTQPTS |
| 26 | GAILGRSETQECLFYNANWELDRTNQTGVEPCEGEKDKRLHCFATWRNISGSIEIV |
| | KKGCWLDDINCYDRTDCVETEENPQVYFCCCEGNMCNEKFSYFPEMEVTQPTS |
| 27 | GAILGRSETQECLFYNANWELDRTNQTGVEPCEGEKDKRLHCFATWKNISGSIEIV |
| | KKGCWLDDFNCYDRTDCVETEENPQVYFCCCEGNMCNEKFSYFPEMEVTQPTS |
| 28 | GAILGRSETQECLFYNANWELERTNQTGVERCEGEKDKRLHCYATWKNISGSIEIV |
| | KKGCWLDDINCYDRTDCVETKENPQVYFCCCEGNMCNEKFSYFPEMEVTQPTS |
| 29 | GAILGRSETQECLFYNANWELERTNQTGVERCEGEKDKRLHCFATWRNISGSIEIV |
| | KKGCWLDDINCYDRTDCVETKENPQVYFCCCEGNMCNEKFSYFPEMEVTQPTS |
| 30 | GAILGRSETQECLFYNANWELERTNQTGVERCEGEKDKRLHCFATWKNISGSIEIV |
| | KKGCWLDDFNCYDRTDCVETKENPQVYFCCCEGNMCNEKFSYFPEMEVTQPTS |
| 31 | GAILGRSETQECLFYNANWELERTNQTGVERCEGEKDKRLHCFATWKNISGSIEIV |
| | KKGCWLDDINCYDRTDCVETEENPQVYFCCCEGNMCNEKFSYFPEMEVTQPTS |
| 32 | GAILGRSETQECLFYNANWELERTNQTGVEPCEGEKDKRLHCYATWRNISGSIEIV |
| | KKGCWLDDINCYDRTDCVETKENPQVYFCCCEGNMCNEKFSYFPEMEVTQPTS |
| 33 | GAILGRSETQECLFYNANWELERTNQTGVEPCEGEKDKRLHCYATWKNISGSIEIV |
| | KKGCWLDDFNCYDRTDCVETKENPQVYFCCCEGNMCNEKFSYFPEMEVTQPTS |
| 34 | GAILGRSETQECLFYNANWELERTNQTGVEPCEGEKDKRLHCYATWKNISGSIEIV |
| | KKGCWLDDINCYDRTDCVETEENPQVYFCCCEGNMCNEKFSYFPEMEVTQPTS |
| 35 | GAILGRSETQECLFYNANWELERTNQTGVEPCEGEKDKRLHCFATWRNISGSIEIV |
| | KKGCWLDDFNCYDRTDCVETKENPQVYFCCCEGNMCNEKFSYFPEMEVTQPTS |
| 36 | GAILGRSETQECLFYNANWELERTNQTGVEPCEGEKDKRLHCFATWRNISGSIEIV |
| | KKGCWLDDINCYDRTDCVETEENPQVYFCCCEGNMCNEKFSYFPEMEVTQPTS |
| 37 | GAILGRSETQECLFYNANWELERTNQTGVEPCEGEKDKRLHCFATWKNISGSIEIV |
| | KKGCWLDDFNCYDRTDCVETEENPQVYFCCCEGNMCNEKFSYFPEMEVTQPTS |
| 38 | GAILGRSETQECLFYNANWELDRTNQTGVERCEGEKDKRLHCYATWRNISGSIEIV |
| | KKGCWLDDINCYDRTDCVETKENPQVYFCCCEGNMCNEKFSYFPEMEVTQPTS |
| 39 | GAILGRSETQECLFYNANWELDRTNQTGVERCEGEKDKRLHCYATWKNISGSIEIV |
| | KKGCWLDDFNCYDRTDCVETKENPQVYFCCCEGNMCNEKFSYFPEMEVTQPTS |
| 40 | GAILGRSETQECLFYNANWELDRTNQTGVERCEGEKDKRLHCYATWKNISGSIEIV |
| | KKGCWLDDINCYDRTDCVETEENPQVYFCCCEGNMCNEKFSYFPEMEVTQPTS |
| 41 | GAILGRSETQECLFYNANWELDRTNQTGVERCEGEKDKRLHCFATWRNISGSIEIV |
| | KKGCWLDDFNCYDRTDCVETKENPQVYFCCCEGNMCNEKFSYFPEMEVTQPTS |
| 42 | GAILGRSETQECLFYNANWELDRTNQTGVERCEGEKDKRLHCFATWRNISGSIEIV |
| | KKGCWLDDINCYDRTDCVETEENPQVYFCCCEGNMCNEKFSYFPEMEVTQPTS |
| 43 | GAILGRSETQECLFYNANWELDRTNQTGVERCEGEKDKRLHCFATWKNISGSIEIV |
| | KKGCWLDDFNCYDRTDCVETEENPQVYFCCCEGNMCNEKFSYFPEMEVTQPTS |
| 44 | GAILGRSETQECLFYNANWELDRTNQTGVEPCEGEKDKRLHCYATWRNISGSIEIV |
| | KKGCWLDDFNCYDRTDCVETKENPQVYFCCCEGNMCNEKFSYFPEMEVTQPTS |
| 45 | GAILGRSETQECLFYNANWELDRTNQTGVEPCEGEKDKRLHCYATWRNISGSIEIV |
| | KKGCWLDDINCYDRTDCVETEENPQVYFCCCEGNMCNEKFSYFPEMEVTQPTS |
| 46 | GAILGRSETQECLFYNANWELDRTNQTGVEPCEGEKDKRLHCYATWKNISGSIEIV |
| | KKGCWLDDFNCYDRTDCVETEENPQVYFCCCEGNMCNEKFSYFPEMEVTQPTS |
| 47 | GAILGRSETQECLFYNANWELDRTNQTGVEPCEGEKDKRLHCFATWRNISGSIEIV |
| | KKGCWLDDFNCYDRTDCVETEENPQVYFCCCEGNMCNEKFSYFPEMEVTQPTS |
| 48 | GAILGRSETQECLFYNANWELERTNQTGVERCEGEKDKRLHCYATWRNISGSIEIV |
| | KKGCWLDDINCYDRTDCVETKENPQVYFCCCEGNMCNEKFSYFPEMEVTQPTS |
| 49 | GAILGRSETQECLFYNANWELERTNQTGVERCEGEKDKRLHCYATWKNISGSIEIV |
| | KKGCWLDDFNCYDRTDCVETKENPQVYFCCCEGNMCNEKFSYFPEMEVTQPTS |
| 50 | GAILGRSETQECLFYNANWELERTNQTGVERCEGEKDKRLHCYATWKNISGSIEIV |
| | KKGCWLDDINCYDRTDCVETEENPQVYFCCCEGNMCNEKFSYFPEMEVTQPTS |
| 51 | GAILGRSETQECLFYNANWELERTNQTGVERCEGEKDKRLHCFATWRNISGSIEIV |
| | KKGCWLDDFNCYDRTDCVETKENPQVYFCCCEGNMCNEKFSYFPEMEVTQPTS |
| 52 | GAILGRSETQECLFYNANWELERTNQTGVERCEGEKDKRLHCFATWRNISGSIEIV |
| | KKGCWLDDINCYDRTDCVETEENPQVYFCCCEGNMCNEKFSYFPEMEVTQPTS |
| 53 | GAILGRSETQECLFYNANWELERTNQTGVERCEGEKDKRLHCFATWKNISGSIEIV |
| | KKGCWLDDFNCYDRTDCVETEENPQVYFCCCEGNMCNEKFSYFPEMEVTQPTS |
| 54 | GAILGRSETQECLFYNANWELERTNQTGVEPCEGEKDKRLHCYATWRNISGSIEIV |
| | KKGCWLDDFNCYDRTDCVETKENPQVYFCCCEGNMCNEKFSYFPEMEVTQPTS |
| 55 | GAILGRSETQECLFYNANWELERTNQTGVEPCEGEKDKRLHCYATWRNISGSIEIV |
| | KKGCWLDDINCYDRTDCVETEENPQVYFCCCEGNMCNEKFSYFPEMEVTQPTS |
| 56 | GAILGRSETQECLFYNANWELERTNQTGVEPCEGEKDKRLHCYATWKNISGSIEIV |
| | KKGCWLDDFNCYDRTDCVETEENPQVYFCCCEGNMCNEKFSYFPEMEVTQPTS |
| 57 | GAILGRSETQECLFYNANWELERTNQTGVEPCEGEKDKRLHCFATWRNISGSIEIV |
| | KKGCWLDDFNCYDRTDCVETEENPQVYFCCCEGNMCNEKFSYFPEMEVTQPTS |
| 58 | GAILGRSETQECLFYNANWELDRTNQTGVERCEGEKDKRLHCYATWRNISGSIEIV |
| | KKGCWLDDFNCYDRTDCVETKENPQVYFCCCEGNMCNEKFSYFPEMEVTQPTS |
| 59 | GAILGRSETQECLFYNANWELDRTNQTGVERCEGEKDKRLHCYATWRNISGSIEIV |
| | KKGCWLDDINCYDRTDCVETEENPQVYFCCCEGNMCNEKFSYFPEMEVTQPTS |
| 60 | GAILGRSETQECLFYNANWELDRTNQTGVERCEGEKDKRLHCYATWKNISGSIEIV |
| | KKGCWLDDFNCYDRTDCVETEENPQVYFCCCEGNMCNEKFSYFPEMEVTQPTS |
| 61 | GAILGRSETQECLFYNANWELDRTNQTGVERCEGEKDKRLHCFATWRNISGSIEIV |
| | KKGCWLDDFNCYDRTDCVETEENPQVYFCCCEGNMCNEKFSYFPEMEVTQPTS |
| 62 | GAILGRSETQECLFYNANWELDRTNQTGVEPCEGEKDKRLHCYATWRNISGSIEIV |
| | KKGCWLDDFNCYDRTDCVETEENPQVYFCCCEGNMCNEKFSYFPEMEVTQPTS |
| 63 | GAILGRSETQECLFYNANWELERTNQTGVERCEGEKDKRLHCYATWRNISGSIEIV |
| | KKGCWLDDFNCYDRTDCVETKENPQVYFCCCEGNMCNEKFSYFPEMEVTQPTS |
| 64 | GAILGRSETQECLFYNANWELERTNQTGVERCEGEKDKRLHCYATWRNISGSIEIV |
| | KKGCWLDDINCYDRTDCVETEENPQVYFCCCEGNMCNEKFSYFPEMEVTQPTS |
| 65 | GAILGRSETQECLFYNANWELERTNQTGVERCEGEKDKRLHCYATWKNISGSIEIV |
| | KKGCWLDDFNCYDRTDCVETEENPQVYFCCCEGNMCNEKFSYFPEMEVTQPTS |
| 66 | GAILGRSETQECLFYNANWELERTNQTGVERCEGEKDKRLHCFATWRNISGSIEIV |
| | KKGCWLDDFNCYDRTDCVETEENPQVYFCCCEGNMCNEKFSYFPEMEVTQPTS |
| 67 | GAILGRSETQECLFYNANWELDRTNQTGVERCEGEKDKRLHCYATWRNISGSIEIV |
| | KKGCWLDDFNCYDRTDCVETEENPQVYFCCCEGNMCNEKFSYFPEMEVTQPTS |
| 68 | GAILGRSETQECLFYNANWELERTNQTGVEPCEGEKDKRLHCYATWRNISGSIEIV |
| | KKGCWLDDFNCYDRTDCVETEENPQVYFCCCEGNMCNEKFSYFPEMEVTQPTS |
| 69 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTS |
| 70 | GAILGRSETQECLYYNANWELERTNQTGVERCEGEQDKRLHCYATWRNISGSIEIV |
| | KKGCWLDDFNCYDRTDCVETEENPQVYFCCCEGNMCNEKFSYFPEMEVTQPTS |
| 71 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTSN |
| | P |
| 72 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTSN |
| | PVTPK |

In some embodiments, a polypeptide described herein including an extracellular ActRIIA variant (e.g., any one of SEQ ID NOs: 1-72 (e.g., SEQ ID NOs: 6-72)) has amino acid K at position X₁₇. Altering the amino acid at position X₁₇ can result in reduced activity. For example, an ActRIIA variant having the sequence GAILGRSETQECLFYNANWELERTNQTGVERCEGEKDKRLHCYATWRNISGSIEIVAKGCWLDDFNCYD RTDCVETEENPQVYFCCCEGNMCNEKFSYFPEMEVTQPTS (SEQ ID NO: 149) has reduced activity in vivo, indicating that the substitution of A for K at X₁₇ is not tolerated.

In some embodiments, a polypeptide described herein including an extracellular ActRIIA variant (e.g., any one of SEQ ID NOs: 1-72 (e.g., SEQ ID NOs: 6-72)) with the sequence TEEN (SEQ ID NO: 374) at positions X₂₃, X₂₄, X₂₅, and X₂₆ can have a substitution of the amino acid K for the amino acid E at position X₂₄. In some embodiments, a polypeptide described herein including an extracellular ActRIIA variant (e.g., any one of SEQ ID NOs: 1-72 (e.g., SEQ ID NOs: 6-72)) with the sequence TKEN (SEQ ID NO: 375) at positions X₂₃, X₂₄, X₂₅, and X₂₆ can have a substitution of the amino acid E for the amino acid K at position X₂₄. Polypeptides having the sequence TEEN (SEQ ID NO: 374) or TKEN (SEQ ID NO: 375) at positions X₂₃, X₂₄, X₂₅, and X₂₆ have reduced or weak binding to BMP9 (e.g., reduced binding to BMP9 compared to BMP9 binding of wild-type ActRIIA).

In some embodiments, a polypeptide described herein including an extracellular ActRIIA variant (e.g., any one of SEQ ID NOs: 1-70 (e.g., SEQ ID NOs: 6-70)) may further include a C-terminal extension (e.g., one more additional amino acids at the C-terminus). The C-terminal extension may correspond to sequence from the same position in wild-type ActRIIA. In some embodiments, the C-terminal extension is amino acid sequence NP. For example, a sequence including the C-terminal extension NP is SEQ ID NO: 71 (e.g., SEQ ID NO: 69 with a C-terminal extension of NP). In some embodiments, the C-terminal extension is amino acid sequence NPVTPK (SEQ ID NO: 154). For example, a sequence including the C-terminal extension NPVTPK is SEQ ID NO: 72 (e.g., SEQ ID NO: 69 with a C-terminal extension of NPVTPK). The C-terminal extension can add one or more amino acids at the C-terminus (e.g., 1, 2, 3, 4, 5, 6 or more additional amino acids).

In some embodiments, a polypeptide described herein including an extracellular ActRIIA variant may further include an Fc domain monomer, which may be fused to the N- or C-terminus (e.g., C-terminus) of the extracellular ActRIIA variant by way of a linker or other covalent bonds. A polypeptide including an extracellular ActRIIA variant fused to an Fc domain monomer may form a dimer (e.g., homodimer or heterodimer) through the interaction between two Fc domain monomers, which combine to form an Fc domain in the dimer.

In some embodiments, an extracellular ActRIIA variant described herein does not have the sequence of any one of SEQ ID NOs: 76-96 shown in Table 3 below.

TABLE 3
Excluded Extracellular ActRIIA Variants

| SEQ ID NO: | Amino Acid Sequence |
|---|---|
| 76 | GAILGRSETQECLFFNANWEKDRTNQTGVEPCYGDKDKRRHCFATWANISGSIEIV |
| | KQGCWLDDINCYDRTDCVEKKDSPEVYFCCCEGNMCNEKFSYFPEMEVTQPTS |
| 77 | GAILGRSETQECLFFNANWAKDRTNQTGVEPCYGDKDKRRHCFATWKNISGSIEIV |
| | KQGCWLDDINCYDRTDCVEKKDSPEVYFCCCEGNMCNEKFSYFPEMEVTQPTS |
| 78 | GAILGRSETQECLFFNANWEKDATNQTGVEPCYGDKDKRRHCFATWKNISGSIEIV |
| | KQGCWLDDINCYDRTDCVEKKDSPEVYFCCCEGNMCNEKFSYFPEMEVTQPTS |
| 79 | GAILGRSETQECLFFNANWEKDRTNQTGVEPCYGDKAKRRHCFATWKNISGSIEIV |
| | KQGCWLDDINCYDRTDCVEKKDSPEVYFCCCEGNMCNEKFSYFPEMEVTQPTS |
| 80 | GAILGRSETQECLFFNANWEKDRTNQTGVEPCYGDKDARRHCFATWKNISGSIEIV |
| | KQGCWLDDINCYDRTDCVEKKDSPEVYFCCCEGNMCNEKFSYFPEMEVTQPTS |
| 81 | GAILGRSETQECLFFNANWEKDRTNQTGVEPCYGDKDKARHCFATWKNISGSIEIV |
| | KQGCWLDDINCYDRTDCVEKKDSPEVYFCCCEGNMCNEKFSYFPEMEVTQPTS |
| 82 | GAILGRSETQECLFFNANWEKDRTNQTGVEPCYGDKDKRRHCFATWKNISGSIEIV |
| | AQGCWLDDINCYDRTDCVEKKDSPEVYFCCCEGNMCNEKFSYFPEMEVTQPTS |
| 83 | GAILGRSETQECLFFNANWEKDRTNQTGVEPCYGDKDKRRHCFATWKNISGSIEIV |
| | YQGCWLDDINCYDRTDCVEKKDSPEVYFCCCEGNMCNEKFSYFPEMEVTQPTS |
| 84 | GAILGRSETQECLFFNANWEKDRTNQTGVEPCYGDKDKRRHCFATWKNISGSIEIV |
| | FQGCWLDDINCYDRTDCVEKKDSPEVYFCCCEGNMCNEKFSYFPEMEVTQPTS |
| 85 | GAILGRSETQECLFFNANWEKDRTNQTGVEPCYGDKDKRRHCFATWKNISGSIEIVI |
| | QGCWLDDINCYDRTDCVEKKDSPEVYFCCCEGNMCNEKFSYFPEMEVTQPTS |
| 86 | GAILGRSETQECLFFNANWEKDRTNQTGVEPCYGDKDKRRHCFATWKNISGSIEIV |
| | KQGCALDDINCYDRTDCVEKKDSPEVYFCCCEGNMCNEKFSYFPEMEVTQPTS |
| 87 | GAILGRSETQECLFFNANWEKDRTNQTGVEPCYGDKDKRRHCFATWKNISGSIEIV |
| | KQGCWADDINCYDRTDCVEKKDSPEVYFCCCEGNMCNEKFSYFPEMEVTQPTS |
| 88 | GAILGRSETQECLFFNANWEKDRTNQTGVEPCYGDKDKRRHCFATWKNISGSIEIV |
| | KQGCWLKDINCYDRTDCVEKKDSPEVYFCCCEGNMCNEKFSYFPEMEVTQPTS |
| 89 | GAILGRSETQECLFFNANWEKDRTNQTGVEPCYGDKDKRRHCFATWKNISGSIEIV |
| | KQGCWLRDINCYDRTDCVEKKDSPEVYFCCCEGNMCNEKFSYFPEMEVTQPTS |
| 90 | GAILGRSETQECLFFNANWEKDRTNQTGVEPCYGDKDKRRHCFATWKNISGSIEIV |
| | KQGCWLADINCYDRTDCVEKKDSPEVYFCCCEGNMCNEKFSYFPEMEVTQPTS |
| 91 | GAILGRSETQECLFFNANWEKDRTNQTGVEPCYGDKDKRRHCFATWKNISGSIEIV |
| | KQGCWLFDINCYDRTDCVEKKDSPEVYFCCCEGNMCNEKFSYFPEMEVTQPTS |
| 92 | GAILGRSETQECLFFNANWEKDRTNQTGVEPCYGDKDKRRHCFATWKNISGSIEIV |
| | KQGCWLGDINCYDRTDCVEKKDSPEVYFCCCEGNMCNEKFSYFPEMEVTQPTS |
| 93 | GAILGRSETQECLFFNANWEKDRTNQTGVEPCYGDKDKRRHCFATWKNISGSIEIV |
| | KQGCWLMDINCYDRTDCVEKKDSPEVYFCCCEGNMCNEKFSYFPEMEVTQPTS |
| 94 | GAILGRSETQECLFFNANWEKDRTNQTGVEPCYGDKDKRRHCFATWKNISGSIEIV |
| | KQGCWLNDINCYDRTDCVEKKDSPEVYFCCCEGNMCNEKFSYFPEMEVTQPTS |
| 95 | GAILGRSETQECLFFNANWEKDRTNQTGVEPCYGDKDKRRHCFATWKNISGSIEIV |
| | KQGCWLIDINCYDRTDCVEKKDSPEVYFCCCEGNMCNEKFSYFPEMEVTQPTS |
| 96 | GAILGRSETQECLFFNANWEKDRTNQTGVEPCYGDKDKRRHCFATWKNISGSIEIV |
| | KQGCWLDDANCYDRTDCVEKKDSPEVYFCCCEGNMCNEKFSYFPEMEVTQPTS |

Furthermore, in some embodiments, a polypeptide described herein (e.g., an ActRIIA variant-Fc fusion protein) has a serum half-life of at least 7 days in humans. The polypeptide including an extracellular ActRIIA variant may bind to activin A with a K_{D} of 10 pM or higher. In some embodiments, the polypeptide including an extracellular ActRIIA variant does not bind to BMP9 or activin A. In some embodiments, the polypeptide including an extracellular ActRIIA variant binds to activin A, activin B, and/or myostatin and exhibits reduced (e.g., weak) binding to BMP9 (e.g., reduced BMP9 binding compared to BMP9 binding of wild-type ActRIIA). In some embodiments, the polypeptide including an extracellular ActRIIA variant that has reduced or weak binding to BMP9 has the sequence TEEN (SEQ ID NO: 374) or TKEN (SEQ ID NO: 375) at positions X₂₃, X₂₄, X₂₅, and X₂₆. In some embodiments, the polypeptide including an extracellular ActRIIA variant does not substantially bind to human BMP9.

In some embodiments, the polypeptide including an extracellular ActRIIA variant may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide including an extracellular ActRIIA variant may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). The polypeptide including an extracellular ActRIIA variant may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

### ActRIIB Variants

In some embodiments, the ActRII variant for use according to the methods described herein is an extracellular ActRIIB variant constructed by introducing amino acid residues of ActRIIA into ActRIIB, or by introducing novel amino acid substitutions into ActRIIB, with the goal of reducing BMP9 binding to prevent or reduce disruption of endogenous BMP9 signaling. The amino acid substitutions may also impart beneficial physiological and pharmacokinetic properties of ActRIIA, such as longer half-life as an Fc fusion protein. The preferred ActRIIB variants also exhibit similar or improved binding to activins and/or myostatin compared to wild-type ActRIIB, which allows them to compete with endogenous activin receptors for ligand binding and reduce or inhibit endogenous activin receptor signaling. In some embodiments, amino acid substitutions may be introduced to an extracellular ActRIIB variant to reduce or remove the binding affinity of the variant to BMP9.

Polypeptides described herein include an extracellular ActRIIB variant having at least one amino acid substitution relative to the wild-type extracellular ActRIIB having the sequence of SEQ ID NO: 74. Possible amino acid substitutions at 28 different positions may be introduced to an extracellular ActRIIB variant (Table 4). An extracellular ActRIIB variant may have one or more (e.g., 1-28, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, 27, or 28) amino acid substitutions relative the sequence of a wild-type extracellular ActRIIB (SEQ ID NO: 74). In some embodiments, an extracellular ActRIIB variant (e.g., an extracellular ActRIIB variant having a sequence of SEQ ID NO: 157) may include amino acid substitutions at all of the 28 positions as listed in Table 4. In some embodiments, an extracellular ActRIIB variant may include amino acid substitutions at a number of positions, e.g., at 3, 4, 5, 6, 7, 8, 9, 10, 12, 15, 16, 18, 20, 22, 24, 26, or 27 out of the 28 positions, as listed in Table 4. In some embodiments, the substitutions are substitutions of an amino acid from ActRIIA into the same position in ActRIIB. In some embodiments, the substitutions are novel changes (e.g., substitutions of amino acids that are not in the corresponding position of ActRIIA, e.g., S48T, I51 L, Q69D, or E70T).

Amino acid substitutions can worsen or improve the activity and/or binding affinity of the ActRIIB variants described herein (e.g., an extracellular ActRIIB variant having the sequence of any one of SEQ ID NOs: 157-171 (e.g., SEQ ID NOs: 158-171)). In some embodiments, the amino acid substitutions worsen the binding affinity of the ActRIIB variants to BMP9 (e.g., the variants have reduced binding to BMP9 relative to wild-type extracellular ActRIIB or have lower binding to BMP9 than to other ActRIIB ligands (e.g., activin A or B, myostatin, or GDF-11)). In some embodiments, the ActRIIB variants have reduced, weak, or no substantial binding to BMP9. In some embodiments, the amino acid substitutions improve the binding affinity of ActRIIB to myostatin, activin A or B, and/or GDF-11 (e.g., the variants have improved binding affinity relative to wild-type extracellular ActRIIB, or bind more strongly to myostatin, activin A or B, or GDF-11 than to BMP9). In some embodiments, the amino acid substitutions reduce the binding affinity of ActRIIB to myostatin, activin A or B, and/or GDF-11 (e.g., the variants have decreased binding affinity relative to wild-type extracellular ActRIIB, or bind more weakly to myostatin, activin A or B, or GDF-11 than to BMP9). In some embodiments, the amino acid substitutions do not substantially change extracellular ActRIIB function (e.g., the ActRIIB variants are functionally equivalent to the wild-type extracellular ActRIIB). In some embodiments, the amino acid substitutions confer an ActRIIA property or activity on the ActRIIB variant (e.g., the ActRIIB variant has a longer half-life as an Fc fusion protein than WT extracellular ActRIIB-Fc). Preferably, the ActRIIB variants have one or more, two or more, or three or more of the above properties (e.g., reduced BMP9 binding and improved binding to activin A or B, myostatin, and/or GDF-11, or reduced BMP9 binding and functional equivalence to wild-type ActRIIB).

The ActRIIB variants described herein (e.g., an extracellular ActRIIB variant having the sequence of any one of SEQ ID NOs: 157-171 (e.g., SEQ ID NOs: 158-171)) preferably have one or more amino acid substitutions that reduce BMP9 binding. In some embodiments, the amino acid substitution that reduces BMP9 binding is E75K (e.g., X₂₄ is K in SEQ ID NO: 157). In some embodiments, the amino acid substitutions that reduce BMP9 binding are Q69T and E70D (e.g., X₂₁ is T and X₂₂ is D in SEQ ID NO: 157). In some embodiments, the amino acid substitutions that reduce BMP9 binding are Q69D and E70T (e.g., X₂₁ is D and X₂₂ is T in SEQ ID NO: 157). In some embodiments, the amino acid substitutions that reduce BMP9 binding are T74K, E75K, E76D, N77S, and Q79E (e.g., X₂₃, X₂₄, X₂₅, X₂₆, and X₂₈ are K, K, D, S, and E, respectively, in SEQ ID NO: 157). In some embodiments, the ActRIIB variants have more than one of the aforementioned amino acid substitutions that reduce BMP9 binding (e.g., substitution E75K and substitutions Q69D and E70T, or substitution E75K and substitutions Q69T and E70D). In some embodiments, the ActRIIB variants described herein have one or more amino acid substitutions that reduce BMP9 binding, and one or more additional amino acid substitutions. The additional amino acid substitutions may confer other beneficial properties, such as altered binding to activins or myostatin or improved activity. For example, amino acid substitutions T74K, E75K, E76D, N77S, and Q79E lead to a reduction in ActRIIB variant activity (e.g., compared to wild-type extracellular ActRIIB), but including additional substitutions S25T and S471; E31Y, E33D, and Q34K; or Y41F, R45K, and K56Q improves the effect of the ActRIIB variant. The additional amino acid substitutions may include one or more of substitutions I11L, Y12F, L19K, E20D, S25T, L27V, R29P, E31Y, E33D, Q34K, L38R, Y41F, R45K, S471, S48T, T50S, I51L, L53I, K56Q and F63I, T74K, E76D, N77S, Q79E, or F89M.

In some embodiments, a polypeptide described herein includes an extracellular ActRIIB variant having the sequence of SEQ ID NO: 157.

TABLE 4
Amino acid substitutions in an extracellular
ActRIIB variant having a sequence of SEQ ID NO: 157

GRGEAETRECX₁X₂YNANWEX₃X₄RTNQX₅GX₆EX₇CX₈GX₉X₁₀DKRX₁₁HC
X₁₂ASWX₁₃NX₁₄X₁₅GX₁₆X₁₇EX₁₈VKX₁₉GCWLDDX₂₀NCYDRX₂₁X₂₂CVAX₂₃
X₂₄X₂₅X₂₆PX₂₇VYFCCCEGNX₂₈CNERFTHLPEAGGPEVTYEPPPTAPT
(SEQ ID NO: 157)

| | | | |
|---|---|---|---|
| X₁ | I or L | X₁₅ | S or T |
| X₂ | F or Y | X₁₆ | S or T |
| X₃ | L or K | X₁₇ | I or L |
| X₄ | D or E | X₁₈ | I or L |
| X₅ | T or S | X₁₉ | K or Q |
| X₆ | L or V | X₂₀ | F or I |
| X₇ | P or R | X₂₁ | Q, T, or D |
| X₈ | Y or E | X₂₂ | E, D, or T |
| X₉ | D or E | X₂₃ | K or T |
| X₁₀ | K or Q | X₂₄ | K or E |
| X₁₁ | R or L | X₂₅ | D or E |
| X₁₂ | Y or F | X₂₆ | S or N |
| X₁₃ | R or K | X₂₇ | E or Q |
| X₁₄ | S or I | X₂₈ | F or M |

In same embodiments, a polypeptide described herein includes an extracellular ActRIIB variant having a sequence of any one of SEQ ID NOs: 158-1 71 (Table 5).

TABLE 5
Extracellular ActRIIB variants having the sequences of SEQ ID NOs: 158-171

| SEQ ID NO: | Amino Acid Sequence |
|---|---|
| 158 | GRGEAETRECIFYNANWEKDRTNQSGLEPCYGDQDKRRHCFASWKNSSGTIELVK |
| | QGCWLDDINCYDRQECVAKKDSPEVYFCCCEGNFCNERFTHLPEAGGPEVTYEP |
| | PPTAPT |
| 159 | GRGEAETRECIYYNANWELDRTNQSGLERCEGEQDKRLHCYASWRNSSGTIELVK |
| | KGCWLDDINCYDRQECVATKENPQVYFCCCEGNFCNERFTHLPEAGGPEVTYEPP |
| | PTAPT |
| 160 | GRGEAETRECIYYNANWELERTNQSGLERCEGEQDKRLHCYASWRNSSGTIELVK |
| | KGCWLDDFNCYDRQECVAKKDSPEVYFCCCEGNFCNERFTHLPEAGGPEVTYEP |
| | PPTAPT |
| 161 | GRGEAETRECIYYNANWELERTNQTGLERCEGEQDKRLHCYASWRNISGTIELVK |
| | KGCWLDDFNCYDRQECVAKKDSPEVYFCCCEGNFCNERFTHLPEAGGPEVTYEP |
| | PPTAPT |
| 162 | GRGEAETRECIYYNANWELERTNQTGLERCEGEQDKRLHCYASWRNITGTIELVKK |
| | GCWLDDFNCYDRQECVAKKDSPEVYFCCCEGNFCNERFTHLPEAGGPEVTYEPP |
| | PTAPT |
| 163 | GRGEAETRECIYYNANWELERTNQSGLEPCEGEQDKRLHCYASWRNSSGTIELVK |
| | KGCWLDDFNCYDRQECVAKKDSPEVYFCCCEGNFCNERFTHLPEAGGPEVTYEP |
| | PPTAPT |
| 164 | GRGEAETRECIYYNANWELERTNQSGLERCYGDKDKRLHCYASWRNSSGTIELVK |
| | KGCWLDDFNCYDRQECVAKKDSPEVYFCCCEGNFCNERFTHLPEAGGPEVTYEP |
| | PPTAPT |
| 165 | GRGEAETRECIYYNANWELERTNQSGLERCEGEQDKRLHCFASWKNSSGTIELVK |
| | QGCWLDDFNCYDRQECVAKKDSPEVYFCCCEGNFCNERFTHLPEAGGPEVTYEP |
| | PPTAPT |
| 166 | GRGEAETRECIFYNANWEKDRTNQSGLERCEGEQDKRLHCYASWRNSSGTIELVK |
| | KGCWLDDFNCYDRQECVAKKDSPEVYFCCCEGNFCNERFTHLPEAGGPEVTYEP |
| | PPTAPT |
| 167 | GRGEAETRECIYYNANWELERTNQSGLERCYGDQDKRRHCYASWRNSSGTIELV |
| | KKGCWLDDFNCYDRQECVAKKDSPEVYFCCCEGNFCNERFTHLPEAGGPEVTYE |
| | PPPTAPT |
| 168 | GRGEAETRECLYYNANWELERTNQSGVERCEGEKDKRLHCYASWRNSSGSLEIV |
| | KKGCWLDDFNCYDRTDCVATEENPQVYFCCCEGNMCNERFTHLPEAGGPEVTYE |
| | PPPTAPT |
| 169 | GRGEAETRECLYYNANWELERTNQSGVERCEGEKDKRLHCYASWRNSSGSLEIV |
| | KKGCWLDDFNCYDRDTCVATEENPQVYFCCCEGNMCNERFTHLPEAGGPEVTYE |
| | PPPTAPT |
| 170 | GRGEAETRECLYYNANWELERTNQSGVERCEGEKDKRLHCYASWRNSSGSLEIV |
| | KKGCWLDDFNCYDRTDCVATKENPQVYFCCCEGNMCNERFTHLPEAGGPEVTYE |
| | PPPTAPT |
| 171 | GRGEAETRECLYYNANWELERTNQSGVERCEGEKDKRLHCYASWRNSSGSLEIV |
| | KKGCWLDDFNCYDRDTCVATKENPQVYFCCCEGNMCNERFTHLPEAGGPEVTYE |
| | PPPTAPT |

In some embodiments, the extracellular ActRIIB variants described herein have an N-terminal truncation of 1-7 amino acids (e.g., 1, 2, 3, 4, 5, 6, or 7 amino acids). The N-terminal truncation can involve the removal of 1-7 amino acids from the N-terminus of any of the ActRIIB variants shown in Tables 4 and 5. The N-terminal truncation can remove amino acids up to two amino acids before the first cysteine (e.g., the two amino acids before the first cysteine (RE) are retained in the N-terminally truncated ActRIIB variants).

In some embodiments, a polypeptide described herein including an extracellular ActRIIB variant may further include an Fc domain monomer, which may be fused to the N- or C-terminus (e.g., C-terminus) of the extracellular ActRIIB variant by way of a linker or other covalent bonds. A polypeptide including an extracellular ActRIIB variant fused to an Fc domain monomer may form a dimer (e.g., homodimer or heterodimer) through the interaction between two Fc domain monomers, which combine to form an Fc domain in the dimer.

Furthermore, in some embodiments, a polypeptide described herein (e.g., an ActRIIB variant-Fc fusion protein) has a serum half-life of at least 7 days in humans. The polypeptide including an extracellular ActRIIB variant may bind to activin A with a K_{D} of 10 pM or higher. In some embodiments, the polypeptide including an extracellular ActRIIB variant does not bind to BMP9 or activin A. In some embodiments, the polypeptide including an extracellular ActRIIB variant binds to activin A, activin B, and/or myostatin and exhibits reduced (e.g., weak) binding to BMP9. In some embodiments, the polypeptide including an extracellular ActRIIB variant does not substantially bind to human BMP9.

In some embodiments, the polypeptide including an extracellular ActRIIB variant may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide including an extracellular ActRIIB variant may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). The polypeptide including an extracellular ActRIIB variant may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or200 pM or higher).

### ActRII Chimeras

In some embodiments, the ActRII variant for use according to the methods described herein is an extracellular ActRII chimera constructed by combining portions of extracellular ActRIIA and ActRIIB with the goal of generating proteins that bind to ActRII ligands (e.g., activin A, activin B, myostatin, and GDF11) and retain the function of wild-type extracellular ActRII proteins. In some embodiments, the ActRII chimeras exhibit reduced BMP9 binding relative to wild-type extracellular ActRIIB, which can prevent or reduce disruption of endogenous BMP9 signaling. In some embodiments, the chimeras have properties of both ActRIIA (e.g., low binding affinity to BMP9 and/or longer serum half-life as an Fc fusion protein) and ActRIIB (e.g., strong binding affinity to activins A and B). The ActRII chimeras may exhibit similar or improved binding to activins (e.g., activin A and/or activin B) and/or myostatin compared to wild-type extracellular ActRIIA and/or ActRIIB, allowing them to compete with endogenous activin receptors for ligand binding and reduce or inhibit endogenous activin receptor signaling.

Polypeptides described herein include an extracellular ActRII chimera containing sequence from both the extracellular portion of ActRIIB and the extracellular portion of ActRIIA. The ActRII chimeras described herein result from joining an N-terminal portion of extracellular ActRIIB (SEQ ID NO: 74 shown above) to a C-terminal portion of extracellular ActRIIA (SEQ ID NO: 73 shown above) such that the sequences are contiguous (e.g., the ActRIIA sequence continues where the ActRIIB sequence left off, starting with the next the amino acid located in the corresponding position of ActRIIA). In some embodiments, the N-terminus of the ActRII chimera includes the six amino acids found at the N-terminus of extracellular ActRIIA joined to the fifth amino acid of extracellular ActRIIB. In some embodiments, the N-terminus of the ActRII chimera begins with the first amino acid located at the N-terminus of extracellular ActRIIB. Accordingly, in some embodiments, the N-terminal portion of ActRIIB begins with the amino acid in the fifth position of SEQ ID NO: 74 (A), while in other embodiments (e.g., in embodiments in which the six amino acids found at the N-terminus of extracellular ActRIIA are not included in the chimera), the N-terminal portion of ActRIIB begins with the amino acid in the first position of SEQ ID NO: 74 (G). In some embodiments, the N-terminus of the ActRII chimera includes the first ten amino acids found at the N-terminus of extracellular ActRIIA joined to the ninth amino acid of extracellular ActRIIB, in which case the N-terminal portion of ActRIIB begins with the amino acid in the ninth position of SEQ ID NO: 74 (E). The extracellular ActRII chimera may also include one or more amino acid substitutions in the portion of the chimera that corresponds to the sequence of ActRIIB compared to wild-type extracellular ActRIIB (e.g., SEQ ID NO: 74 shown above) and/or one or more amino acid substitutions in the portion of the chimera that corresponds to the sequence of ActRIIA compared to wild-type extracellular ActRIIA (e.g., SEQ ID NO: 73 shown above). Amino acid substitutions at 9 different positions may be introduced into an extracellular ActRII chimera (Table 6). An extracellular ActRII chimera may have one or more (e.g., 1, 2, 3, 4, 5, 6, 7, 8, or 9) amino acid substitutions relative the sequence of a wild-type sequence (e.g., relative to the sequence of wild-type extracellular ActRIIB (SEQ ID NO: 74) if the portion of the chimera corresponds to a region of wild-type extracellular ActRIIB, or relative to the sequence of wild-type extracellular ActRIIA (SEQ ID NO: 73) if the portion of the chimera corresponds to a region of wild-type extracellular ActRIIA). The positions at which amino acid substitutions may be made, as well as the amino acids that may be substituted at these positions, are listed in Table 6.

Amino acid substitutions can alter the activity and/or binding affinity of the extracellular ActRII chimeras described herein. In some embodiments, the extracellular ActRII chimeras bind to activin A, activin B, myostatin, and/or GDF11 with sufficient affinity to compete with endogenous activin receptors for binding to one or more of these ligands. In some embodiments, the extracellular ActRII chimeras described herein have reduced, weak, or no substantial binding to BMP9 (e.g., compared to wild-type ActRIIB). BMP9 binding may be reduced in extracellular ActRII chimeras containing the amino acid sequence TEEN (SEQ ID NO: 374) or TKEN (SEQ ID NO: 375) at positions X₃, X₄, X₅, and X₆. In some embodiments, a polypeptide described herein including an extracellular ActRII chimera (e.g., any one of SEQ ID NOs: 174-216 (e.g., SEQ ID NOs: 195-216)) with the sequence TEEN (SEQ ID NO: 374) at positions X₃, X₄, X₅, and Xe can have a substitution of the amino acid K for the amino acid E at position X₄. In some embodiments, a polypeptide described herein including an extracellular ActRII chimera (e.g., any one of SEQ ID NOs: 174-216 (e.g., SEQ ID NOs: 195-216)) with the sequence TKEN (SEQ ID NO: 375) at positions X₃, X₄, X₅, and Xe can have a substitution of the amino acid E for the amino acid K at position X₄. The sequences TEEN (SEQ ID NO: 374) and TKEN (SEQ ID NO: 375) can be used interchangeably in the extracellular ActRII chimeras (e.g., the chimeras in Tables 6 and 7, e.g., SEQ ID NOs: 174-216 (e.g., SEQ ID NOs: 195-216)) described herein.

The extracellular ActRII chimeras described herein may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the chimeras shown in Tables 6 and 7 (e.g., SEQ ID NOs: 174-216 (e.g., SEQ ID NOs: 195-216)). The C-terminal extension may correspond to sequence from the same position in wild-type ActRIIA or ActRIIB. For example, C-terminal extensions that can be included in the extracellular ActRII chimeras described herein are the amino acid sequence NP and the amino acid sequence NPVTPK (SEQ ID NO: 154), which correspond to sequence found in the same position in wild-type ActRIIA.

TABLE 6
Amino acid substitutions in an extracellular ActRII chimera having a
sequence of any one of SEQ ID NOs: 174-194

GAILGRAETRECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 174)
GAILGRAETRECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 175)
GAILGRAETRECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 176)
GAILGRAETRECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 177)
GAILGRAETRECIYYNANWELERTNQSGLERCEGEQX₁KRLHCYASWRNSSGTIEIVKQGCWLDDX₂X₃
CYDRTDCVX₄X₅X₆X₇X₈PX₉VYFCCCEGNMCNEKFSYFPEMEVTQPTS (SEQ ID NO: 178)
GAILGRAETRECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 179)
GAILGRAETRECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 180)
GRGEAETRECIYYNANWELERTNQSGLERCEGEQX₁KRRHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 181)
GRGEAETRECIYYNANWELERTNQSGLERCEGEQX₁KRLHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 182)
GRGEAETRECIYYNANWELERTNQSGLERCEGEQX₁KRLHCYASWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 183)
GRGEAETRECIYYNANWELERTNQSGLERCEGEQX₁KRLHCYASWRNSSGSIEIVKQGCWLDDX₂X₃C
YDRTDCVX₄X₅X₆X₇X₈PX₉VYFCCCEGNMCNEKFSYFPEMEVTQPTS (SEQ ID NO: 184)
GRGEAETRECIYYNANWELERTNQSGLERCEGEQX₁KRLHCYASWRNSSGTIEIVKQGCWLDDX₂X₃C
YDRTDCVX₄X₅X₆X₇X₈PX₉VYFCCCEGNMCNEKFSYFPEMEVTQPTS (SEQ ID NO: 185)
GRGEAETRECIYYNANWELERTNQSGLERCEGEQX₁KRLHCYASWRNSSGTIELVKKGCWLDDX₂X₃
CYDRTDCVX₄X₅X₆X₇X₈PX₉VYFCCCEGNMCNEKFSYFPEMEVTQPTS (SEQ ID NO: 186)
GRGEAETRECIYYNANWELERTNQSGLERCEGEQX₁KRLHCYASWRNSSGTIELVKKGCWLDDX₂X₃
CYDRQECVX₄X₅X₆X₇X₈PX₉VYFCCCEGNMCNEKFSYFPEMEVTQPTS (SEQ ID NO: 187)
GAILGRSETQECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 188)
GAILGRSETQECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 189)
GAILGRSETQECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 190)
GAILGRSETQECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 191)
GAILGRSETQECIYYNANWELERTNQSGLERCEGEQX₁KRLHCYASWRNSSGTIEIVKQGCWLDDX₂
X₃CYDRTDCVX₄X₅X₆X₇X₈PX₉VYFCCCEGNMCNEKFSYFPEMEVTQPTS (SEQ ID NO: 192)
GAILGRSETQECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 193)
GAILGRSETQECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 194)

| | | | |
|---|---|---|---|
| X₁ | D or R | X₆ | E or K |
| X₂ | I, F, E, D, Y, S, | X₇ | E or D |
| | N, Q, or T | | |
| X₃ | N or T | X₈ | N or S |
| X₄ | A or E | X₉ | Q, E, K, R, D, or N |
| X₅ | T or K | | |

In some embodiments, in the extracellular ActRII chimeras of SEQ ID NOs: 174-216 (shown in Table 6), X₁₁ is D, X₂ is, F, or E, X₃ is N or T, X₄ is A or E, X is T or K, X₆ is E or K, X₇ is E or D, X₈ is N or S, and X₉ is E or Q. In some embodiments, in the extracellular ActRII chimeras of SEQ ID NOs: 174-216, X₁ is D, X₂ is I or F, X₃ is N, X₄ is A or E, X₅ is T or K, X₆ is E or K, X₇ is E or D, X₈ is N or S, and X₉ is E or Q.

In some embodiments, a polypeptide described herein includes an extracellular ActRII chimera having the sequence of any one of SEQ ID NOs: 195-216 (Table 7).

TABLE 7
Extracellular ActRII chimeras having the sequences of SEQ ID NOs: 195-216

| SEQ ID NO: | Amino Acid Sequence |
|---|---|
| 195 | GAILGRAETRECIYYNANWELERTNQSGLERCEGEQDKRLHCYASWRNSSGTIEIV |
| | KQGCWLDDINCYDRTDCVATEENPQVYFCCCEGNMCNEKFSYFPEMEVTQPTS |
| 196 | GRGEAETRECIYYNANWELERTNQSGLERCEGEQDKRLHCYASWRNSSGTIEIVK |
| | QGCWLDDINCYDRTDCVEKKDSPEVYFCCCEGNMCNEKFSYFPEMEVTQPTS |
| 197 | GRGEAETRECIYYNANWELERTNQSGLERCEGEQDKRLHCYASWRNSSGTIEIVK |
| | QGCWLDDINCYDRTDCVEKKDSPQVYFCCCEGNMCNEKFSYFPEMEVTQPTS |
| 198 | GRGEAETRECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTS |
| 199 | GAILGRAETRECIYYNANWELERTNQSGLERCEGEQDKRLHCYASWRNSSGTIEIV |
| | KQGCWLDDFNCYDRTDCVATEENPQVYFCCCEGNMCNEKFSYFPEMEVTQPTS |
| 200 | GRGEAETRECIYYNANWELERTNQSGLERCEGEQDKRLHCYASWRNSSGTIEIVK |
| | QGCWLDDFNCYDRTDCVEKKDSPEVYFCCCEGNMCNEKFSYFPEMEVTQPTS |
| 201 | GRGEAETRECIYYNANWELERTNQSGLERCEGEQDKRRHCFATWKNISGSIEIVKQ |
| | GCWLDDFNCYDRTDCVEKKDSPQVYFCCCEGNMCNEKFSYFPEMEVTQPTS |
| 202 | GRGEAETRECIYYNANWELERTNQSGLERCEGEQDKRLHCFATWKNISGSIEIVKQ |
| | GCWLDDFNCYDRTDCVEKKDSPQVYFCCCEGNMCNEKFSYFPEMEVTQPTS |
| 203 | GRGEAETRECIYYNANWELERTNQSGLERCEGEQDKRLHCYASWKNISGSIEIVKQ |
| | GCWLDDFNCYDRTDCVEKKDSPQVYFCCCEGNMCNEKFSYFPEMEVTQPTS |
| 204 | GRGEAETRECIYYNANWELERTNQSGLERCEGEQDKRLHCYASWRNSSGSIEIVK |
| | QGCWLDDFNCYDRTDCVEKKDSPQVYFCCCEGNMCNEKFSYFPEMEVTQPTS |
| 205 | GRGEAETRECIYYNANWELERTNQSGLERCEGEQDKRLHCYASWRNSSGTIELVK |
| | KGCWLDDFNCYDRTDCVEKKDSPQVYFCCCEGNMCNEKFSYFPEMEVTQPTS |
| 206 | GRGEAETRECIYYNANWELERTNQSGLERCEGEQDKRLHCYASWRNSSGTIELVK |
| | KGCWLDDFNCYDRQECVATKDSPQVYFCCCEGNMCNEKFSYFPEMEVTQPTS |
| 207 | GRGEAETRECIYYNANWELERTNQSGLERCEGEQDKRLHCYASWRNSSGTIELVK |
| | KGCWLDDFNCYDRQECVATEENPQVYFCCCEGNMCNEKFSYFPEMEVTQPTS |
| 208 | GRGEAETRECIYYNANWELERTNQSGLERCEGEQDKRLHCYASWRNSSGTIEIVK |
| | QGCWLDDNNCYDRTDCVEKKDSPQVYFCCCEGNMCNEKFSYFPEMEVTQPTS |
| 209 | GRGEAETRECIYYNANWELERTNQSGLERCEGEQDKRLHCYASWRNSSGTIEIVK |
| | QGCWLDDTNCYDRTDCVEKKDSPQVYFCCCEGNMCNEKFSYFPEMEVTQPTS |
| 210 | GRGEAETRECIYYNANWELERTNQSGLERCEGEQDKRLHCYASWRNSSGTIEIVK |
| | QGCWLDDETCYDRTDCVEKKDSPQVYFCCCEGNMCNEKFSYFPEMEVTQPTS |
| 211 | GRGEAETRECIYYNANWELERTNQSGLERCEGEQRKRLHCYASWRNSSGTIEIVK |
| | QGCWLDDFNCYDRTDCVEKKDSPQVYFCCCEGNMCNEKFSYFPEMEVTQPTS |
| 212 | GRGEAETRECIYYNANWELERTNQSGLERCEGEQDKRLHCYASWRNSSGTIEIVK |
| | QGCWLDDFNCYDRTDCVETKDSPQVYFCCCEGNMCNEKFSYFPEMEVTQPTS |
| 213 | GAILGRAETRECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 214 | GAILGRSETQECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 215 | GRGEAETRECIYYNANWELERTNQSGLERCEGEQDKRLHCYASWRNSSGTIELVK |
| | KGCWLDDFNCYDRQECVATKDSPEVYFCCCEGNMCNEKFSYFPEMEVTQPTS |
| 216 | GRGEAETRECIYYNANWELERTNQSGLERCEGEQDKRLHCYASWRNSSGTIELVK |
| | KGCWLDDFNCYDRQECVATKENPQVYFCCCEGNMCNEKFSYFPEMEVTQPTS |

In some embodiments, the extracellular ActRII chimeras described herein have an N-terminal truncation of 1-9 amino acids (e.g., 1, 2, 3, 4, 5, 6, 7, 8, or 9 amino acids). The N-terminal truncation can involve the removal of 1-9 amino acids from the N-terminus of any of the chimeras shown in Tables 6 and 7 (e.g., SEQ ID NOs: 174-216 (e.g., SEQ ID NOs: 195-216)). The N-terminal truncation can remove amino acids up to two amino acids before the first cysteine (e.g., the two amino acids before the first cysteine (RE or QE) are retained in the N-terminally truncated ActRII chimeras). Exemplary ActRII chimeras having N-terminal truncations are provided in Table 8, below.

TABLE 8
Extracellular ActRII chimeras having N-terminal truncations

| SEQ ID NO: | Amino Acid Sequence |
|---|---|
| 221 | ILGRAETRECIYYNANWELERTNQSGLERCEGEQDKRLHCYASWRNSSGTIEIVKQ |
| | GCWLDDINCYDRTDCVATEENPQVYFCCCEGNMCNEKFSYFPEMEVTQPTS |
| 222 | RGEAETRECIYYNANWELERTNQSGLERCEGEQDKRLHCYASWRNSSGTIEIVKQ |
| | GCWLDDINCYDRTDCVEKKDSPEVYFCCCEGNMCNEKFSYFPEMEVTQPTS |
| 223 | RGEAETRECIYYNANWELERTNQSGLERCEGEQDKRLHCYASWRNSSGTIEIVKQ |
| | GCWLDDINCYDRTDCVEKKDSPQVYFCCCEGNMCNEKFSYFPEMEVTQPTS |
| 224 | RGEAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 225 | LGRAETRECIYYNANWELERTNQSGLERCEGEQDKRLHCYASWRNSSGTIEIVKQ |
| | GCWLDDINCYDRTDCVATEENPQVYFCCCEGNMCNEKFSYFPEMEVTQPTS |
| 226 | GEAETRECIYYNANWELERTNQSGLERCEGEQDKRLHCYASWRNSSGTIEIVKQG |
| | CWLDDINCYDRTDCVEKKDSPEVYFCCCEGNMCNEKFSYFPEMEVTQPTS |
| 227 | GEAETRECIYYNANWELERTNQSGLERCEGEQDKRLHCYASWRNSSGTIEIVKQG |
| | CWLDDINCYDRTDCVEKKDSPQVYFCCCEGNMCNEKFSYFPEMEVTQPTS |
| 228 | GEAETRECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTS |
| 229 | GRAETRECIYYNANWELERTNQSGLERCEGEQDKRLHCYASWRNSSGTIEIVKQG |
| | CWLDDINCYDRTDCVATEENPQVYFCCCEGNMCNEKFSYFPEMEVTQPTS |
| 230 | EAETRECIYYNANWELERTNQSGLERCEGEQDKRLHCYASWRNSSGTIEIVKQGC |
| | WLDDINCYDRTDCVEKKDSPEVYFCCCEGNMCNEKFSYFPEMEVTQPTS |
| 231 | EAETRECIYYNANWELERTNQSGLERCEGEQDKRLHCYASWRNSSGTIEIVKQGC |
| | WLDDINCYDRTDCVEKKDSPQVYFCCCEGNMCNEKFSYFPEMEVTQPTS |
| 232 | EAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTS |
| 233 | RAETRECIYYNANWELERTNQSGLERCEGEQDKRLHCYASWRNSSGTIEIVKQGC |
| | WLDDINCYDRTDCVATEENPQVYFCCCEGNMCNEKFSYFPEMEVTQPTS |
| 234 | AETRECIYYNANWELERTNQSGLERCEGEQDKRLHCYASWRNSSGTIEIVKQGCW |
| | LDDINCYDRTDCVEKKDSPEVYFCCCEGNMCNEKFSYFPEMEVTQPTS |
| 235 | AETRECIYYNANWELERTNQSGLERCEGEQDKRLHCYASWRNSSGTIEIVKQGCW |
| | LDDINCYDRTDCVEKKDSPQVYFCCCEGNMCNEKFSYFPEMEVTQPTS |
| 236 | AETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTS |
| 237 | AETRECIYYNANWELERTNQSGLERCEGEQDKRLHCYASWRNSSGTIEIVKQGCW |
| | LDDINCYDRTDCVATEENPQVYFCCCEGNMCNEKFSYFPEMEVTQPTS |
| 238 | ETRECIYYNANWELERTNQSGLERCEGEQDKRLHCYASWRNSSGTIEIVKQGCWL |
| | DDINCYDRTDCVEKKDSPEVYFCCCEGNMCNEKFSYFPEMEVTQPTS |
| 239 | ETRECIYYNANWELERTNQSGLERCEGEQDKRLHCYASWRNSSGTIEIVKQGCWL |
| | DDINCYDRTDCVEKKDSPQVYFCCCEGNMCNEKFSYFPEMEVTQPTS |
| 240 | ETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTS |
| 241 | ETRECIYYNANWELERTNQSGLERCEGEQDKRLHCYASWRNSSGTIEIVKQGCWL |
| | DDINCYDRTDCVATEENPQVYFCCCEGNMCNEKFSYFPEMEVTQPTS |
| 242 | TRECIYYNANWELERTNQSGLERCEGEQDKRLHCYASWRNSSGTIEIVKQGCWLD |
| | DINCYDRTDCVEKKDSPEVYFCCCEGNMCNEKFSYFPEMEVTQPTS |
| 243 | TRECIYYNANWELERTNQSGLERCEGEQDKRLHCYASWRNSSGTIEIVKQGCWLD |
| | DINCYDRTDCVEKKDSPQVYFCCCEGNMCNEKFSYFPEMEVTQPTS |
| 244 | TRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTS |
| 245 | TRECIYYNANWELERTNQSGLERCEGEQDKRLHCYASWRNSSGTIEIVKQGCWLD |
| | DINCYDRTDCVATEENPQVYFCCCEGNMCNEKFSYFPEMEVTQPTS |
| 246 | RECIYYNANWELERTNQSGLERCEGEQDKRLHCYASWRNSSGTIEIVKQGCWLDD |
| | INCYDRTDCVEKKDSPEVYFCCCEGNMCNEKFSYFPEMEVTQPTS |
| 247 | RECIYYNANWELERTNQSGLERCEGEQDKRLHCYASWRNSSGTIEIVKQGCWLDD |
| | INCYDRTDCVEKKDSPQVYFCCCEGNMCNEKFSYFPEMEVTQPTS |
| 248 | RECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTS |
| 249 | RECIYYNANWELERTNQSGLERCEGEQDKRLHCYASWRNSSGTIEIVKQGCWLDD |
| | INCYDRTDCVATEENPQVYFCCCEGNMCNEKFSYFPEMEVTQPTS |
| 250 | RGEAETRECIYYNANWELERTNQSGLERCEGEQDKRRHCFATWKNISGSIEIVKQG |
| | CWLDDFNCYDRTDCVEKKDSPQVYFCCCEGNMCNEKFSYFPEMEVTQPTS |
| 251 | RGEAETRECIYYNANWELERTNQSGLERCEGEQDKRLHCYASWRNSSGTIEIVKQ |
| | GCWLDDETCYDRTDCVEKKDSPQVYFCCCEGNMCNEKFSYFPEMEVTQPTS |
| 252 | ILGRAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 253 | ILGRSETQECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 254 | RGEAETRECIYYNANWELERTNQSGLERCEGEQDKRLHCYASWRNSSGTIELVKK |
| | GCWLDDFNCYDRQECVATKDSPEVYFCCCEGNMCNEKFSYFPEMEVTQPTS |
| 255 | RGEAETRECIYYNANWELERTNQSGLERCEGEQDKRLHCYASWRNSSGTIELVKK |
| | GCWLDDFNCYDRQECVATKENPQVYFCCCEGNMCNEKFSYFPEMEVTQPTS |
| 256 | GEAETRECIYYNANWELERTNQSGLERCEGEQDKRRHCFATWKNISGSIEIVKQGC |
| | WLDDFNCYDRTDCVEKKDSPQVYFCCCEGNMCNEKFSYFPEMEVTQPTS |
| 257 | GEAETRECIYYNANWELERTNQSGLERCEGEQDKRLHCYASWRNSSGTIEIVKQG |
| | CWLDDETCYDRTDCVEKKDSPQVYFCCCEGNMCNEKFSYFPEMEVTQPTS |
| 258 | LGRAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 259 | LGRSETQECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 260 | GEAETRECIYYNANWELERTNQSGLERCEGEQDKRLHCYASWRNSSGTIELVKKG |
| | CWLDDFNCYDRQECVATKDSPEVYFCCCEGNMCNEKFSYFPEMEVTQPTS |
| 261 | GEAETRECIYYNANWELERTNQSGLERCEGEQDKRLHCYASWRNSSGTIELVKKG |
| | CWLDDFNCYDRQECVATKENPQVYFCCCEGNMCNEKFSYFPEMEVTQPTS |
| 262 | EAETRECIYYNANWELERTNQSGLERCEGEQDKRRHCFATWKNISGSIEIVKQGC |
| | WLDDFNCYDRTDCVEKKDSPQVYFCCCEGNMCNEKFSYFPEMEVTQPTS |
| 263 | EAETRECIYYNANWELERTNQSGLERCEGEQDKRLHCYASWRNSSGTIEIVKQGC |
| | WLDDETCYDRTDCVEKKDSPQVYFCCCEGNMCNEKFSYFPEMEVTQPTS |
| 264 | GRAETRECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTS |
| 265 | GRSETQECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTS |
| 266 | EAETRECIYYNANWELERTNQSGLERCEGEQDKRLHCYASWRNSSGTIELVKKGC |
| | WLDDFNCYDRQECVATKDSPEVYFCCCEGNMCNEKFSYFPEMEVTQPTS |
| 267 | EAETRECIYYNANWELERTNQSGLERCEGEQDKRLHCYASWRNSSGTIELVKKGC |
| | WLDDFNCYDRQECVATKENPQVYFCCCEGNMCNEKFSYFPEMEVTQPTS |
| 268 | AETRECIYYNANWELERTNQSGLERCEGEQDKRRHCFATWKNISGSIEIVKQGCW |
| | LDDFNCYDRTDCVEKKDSPQVYFCCCEGNMCNEKFSYFPEMEVTQPTS |
| 269 | AETRECIYYNANWELERTNQSGLERCEGEQDKRLHCYASWRNSSGTIEIVKQGCW |
| | LDDETCYDRTDCVEKKDSPQVYFCCCEGNMCNEKFSYFPEMEVTQPTS |
| 270 | RAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTS |
| 271 | RSETQECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTS |
| 272 | AETRECIYYNANWELERTNQSGLERCEGEQDKRLHCYASWRNSSGTIELVKKGCW |
| | LDDFNCYDRQECVATKDSPEVYFCCCEGNMCNEKFSYFPEMEVTQPTS |
| 273 | AETRECIYYNANWELERTNQSGLERCEGEQDKRLHCYASWRNSSGTIELVKKGCW |
| | LDDFNCYDRQECVATKENPQVYFCCCEGNMCNEKFSYFPEMEVTQPTS |
| 274 | ETRECIYYNANWELERTNQSGLERCEGEQDKRRHCFATWKNISGSIEIVKQGCWL |
| | DDFNCYDRTDCVEKKDSPQVYFCCCEGNMCNEKFSYFPEMEVTQPTS |
| 275 | ETRECIYYNANWELERTNQSGLERCEGEQDKRLHCYASWRNSSGTIEIVKQGCWL |
| | DDETCYDRTDCVEKKDSPQVYFCCCEGNMCNEKFSYFPEMEVTQPTS |
| 276 | AETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTS |
| 277 | SETQECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTS |
| 278 | ETRECIYYNANWELERTNQSGLERCEGEQDKRLHCYASWRNSSGTIELVKKGCWL |
| | DDFNCYDRQECVATKDSPEVYFCCCEGNMCNEKFSYFPEMEVTQPTS |
| 279 | ETRECIYYNANWELERTNQSGLERCEGEQDKRLHCYASWRNSSGTIELVKKGCWL |
| | DDFNCYDRQECVATKENPQVYFCCCEGNMCNEKFSYFPEMEVTQPTS |
| 280 | TRECIYYNANWELERTNQSGLERCEGEQDKRRHCFATWKNISGSIEIVKQGCWLD |
| | DFNCYDRTDCVEKKDSPQVYFCCCEGNMCNEKFSYFPEMEVTQPTS |
| 281 | TRECIYYNANWELERTNQSGLERCEGEQDKRLHCYASWRNSSGTIEIVKQGCWLD |
| | DETCYDRTDCVEKKDSPQVYFCCCEGNMCNEKFSYFPEMEVTQPTS |
| 282 | ETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTS |
| 283 | ETQECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTS |
| 284 | TRECIYYNANWELERTNQSGLERCEGEQDKRLHCYASWRNSSGTIELVKKGCWLD |
| | DFNCYDRQECVATKDSPEVYFCCCEGNMCNEKFSYFPEMEVTQPTS |
| 285 | TRECIYYNANWELERTNQSGLERCEGEQDKRLHCYASWRNSSGTIELVKKGCWLD |
| | DFNCYDRQECVATKENPQVYFCCCEGNMCNEKFSYFPEMEVTQPTS |
| 286 | RECIYYNANWELERTNQSGLERCEGEQDKRRHCFATWKNISGSIEIVKQGCWLDD |
| | FNCYDRTDCVEKKDSPQVYFCCCEGNMCNEKFSYFPEMEVTQPTS |
| 287 | RECIYYNANWELERTNQSGLERCEGEQDKRLHCYASWRNSSGTIEIVKQGCWLDD |
| | ETCYDRTDCVEKKDSPQVYFCCCEGNMCNEKFSYFPEMEVTQPTS |
| 288 | TRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTS |
| 289 | TQECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTS |
| 290 | RECIYYNANWELERTNQSGLERCEGEQDKRLHCYASWRNSSGTIELVKKGCWLD |
| | DFNCYDRQECVATKDSPEVYFCCCEGNMCNEKFSYFPEMEVTQPTS |
| 291 | RECIYYNANWELERTNQSGLERCEGEQDKRLHCYASWRNSSGTIELVKKGCWLD |
| | DFNCYDRQECVATKENPQVYFCCCEGNMCNEKFSYFPEMEVTQPTS |
| 292 | RECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTS |
| 293 | QECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTS |

In some embodiments, a polypeptide described herein including an extracellular ActRII chimera may further include an Fc domain monomer, which may be fused to the N- or C-terminus (e.g., C-terminus) of the extracellular ActRII chimera by way of a linker or other covalent bonds. A polypeptide including an extracellular ActRII chimera fused to an Fc domain monomer may form a dimer (e.g., homodimer or heterodimer) through the interaction between two Fc domain monomers, which combine to form an Fc domain in the dimer. Exemplary polypeptides containing an ActRII chimera, an Fc domain monomer, and a linker are provided in Table 9, below. In some embodiments, the terminal lysine is absent from the Fc domain monomer amino acid sequence.

TABLE 9
Polypeptides containing an extracellular ActRII chimera fused to an Fc domain
monomer by way of a linker

| SEQ ID | |
|---|---|
| NO: | Amino Acid Sequence |
| 217 | GAILGRAETRECIYYNANWELERTNQSGLERCEGEQDKRLHCYASWRNSSGTIEIVKQGC |
| | WLDDINCYDRTDCVATEENPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 218 | GRGEAETRECIYYNANWELERTNQSGLERCEGEQDKRLHCYASWRNSSGTIEIVKQGCW |
| | LDDINCYDRTDCVEKKDSPEVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 219 | GRGEAETRECIYYNANWELERTNQSGLERCEGEQDKRLHCYASWRNSSGTIEIVKQGCW |
| | LDDINCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 220 | GRGEAETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| 294 | ILGRAETRECIYYNANWELERTNQSGLERCEGEQDKRLHCYASWRNSSGTIEIVKQGCWL |
| | DDINCYDRTDCVATEENPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 295 | RGEAETRECIYYNANWELERTNQSGLERCEGEQDKRLHCYASWRNSSGTIEIVKQGCWL |
| | DDINCYDRTDCVEKKDSPEVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 296 | RGEAETRECIYYNANWELERTNQSGLERCEGEQDKRLHCYASWRNSSGTIEIVKQGCWL |
| | DDINCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 297 | RGEAETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 298 | LGRAETRECIYYNANWELERTNQSGLERCEGEQDKRLHCYASWRNSSGTIEIVKQGCWL |
| | DDINCYDRTDCVATEENPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 299 | GEAETRECIYYNANWELERTNQSGLERCEGEQDKRLHCYASWRNSSGTIEIVKQGCWLD |
| | DINCYDRTDCVEKKDSPEVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 300 | GEAETRECIYYNANWELERTNQSGLERCEGEQDKRLHCYASWRNSSGTIEIVKQGCWLD |
| | DINCYDRTDCVEKKDSPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 301 | GEAETRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 302 | GRAETRECIYYNANWELERTNQSGLERCEGEQDKRLHCYASWRNSSGTIEIVKQGCWLD |
| | DINCYDRTDCVATEENPQVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 303 | EAETRECIYYNANWELERTNQSGLERCEGEQDKRLHCYASWRNSSGTIEIVKQGCWLDDI |
| | NCYDRTDCVEKKDSPEVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 304 | EAETRECIYYNANWELERTNQSGLERCEGEQDKRLHCYASWRNSSGTIEIVKQGCWLDDI |
| | NCYDRTDCVEKKDSPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 305 | EAETRECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 306 | RAETRECIYYNANWELERTNQSGLERCEGEQDKRLHCYASWRNSSGTIEIVKQGCWLDDI |
| | NCYDRTDCVATEENPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 307 | AETRECIYYNANWELERTNQSGLERCEGEQDKRLHCYASWRNSSGTIEIVKQGCWLDDIN |
| | CYDRTDCVEKKDSPEVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 308 | AETRECIYYNANWELERTNQSGLERCEGEQDKRLHCYASWRNSSGTIEIVKQGCWLDDIN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 309 | AETRECIYYNANWELERTNQSGLERCEGEQDKRLHCYASWRNSSGTIEIVKQGCWLDDF |
| | NCYDRTDCVEKKDSPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 310 | AETRECIYYNANWELERTNQSGLERCEGEQDKRLHCYASWRNSSGTIEIVKQGCWLDDIN |
| | CYDRTDCVATEENPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 311 | ETRECIYYNANWELERTNQSGLERCEGEQDKRLHCYASWRNSSGTIEIVKQGCWLDDIN |
| | CYDRTDCVEKKDSPEVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 312 | ETRECIYYNANWELERTNQSGLERCEGEQDKRLHCYASWRNSSGTIEIVKQGCWLDDIN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 313 | ETRECIYYNANWELERTNQSGLERCEGEQDKRLHCYASWRNSSGTIEIVKQGCWLDDEN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 314 | ETRECIYYNANWELERTNQSGLERCEGEQDKRLHCYASWRNSSGTIEIVKQGCWLDDIN |
| | CYDRTDCVATEENPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 315 | TRECIYYNANWELERTNQSGLERCEGEQDKRLHCYASWRNSSGTIEIVKQGCWLDDINC |
| | YDRTDCVEKKDSPEVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 316 | TRECIYYNANWELERTNQSGLERCEGEQDKRLHCYASWRNSSGTIEIVKQGCWLDDINC |
| | YDRTDCVEKKDSPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 317 | TRECIYYNANWELERTNQSGLERCEGEQDKRLHCYASWRNSSGTIEIVKQGCWLDDFNC |
| | YDRTDCVEKKDSPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 318 | TRECIYYNANWELERTNQSGLERCEGEQDKRLHCYASWRNSSGTIEIVKQGCWLDDINC |
| | YDRTDCVATEENPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 319 | RECIYYNANWELERTNQSGLERCEGEQDKRLHCYASWRNSSGTIEIVKQGCWLDDINCY |
| | DRTDCVEKKDSPEVYFCCCEGNMCNEKFSYFPEMEVTQPTSGGGDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 320 | RECIYYNANWELERTNQSGLERCEGEQDKRLHCYASWRNSSGTIEIVKQGCWLDDINCY |
| | DRTDCVEKKDSPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 321 | RECIYYNANWELERTNQSGLERCEGEQDKRLHCYASWRNSSGTIEIVKQGCWLDDENCY |
| | DRTDCVEKKDSPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 322 | RECIYYNANWELERTNQSGLERCEGEQDKRLHCYASWRNSSGTIEIVKQGCWLDDINCY |
| | DRTDCVATEENPQVYFCCCEGNMCNEKFSYFPEMEVTQPTSGGGDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 323 | GRGEAETRECIYYNANWELERTNQSGLERCEGEQDKRRHCFATWKNISGSIEIVKQGCW |
| | LDDFNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| 324 | GRGEAETRECIYYNANWELERTNQSGLERCEGEQDKRLHCYASWRNSSGTIEIVKQGCW |
| | LDDETCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| 325 | GAILGRAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 326 | GAILGRSETQECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 327 | GRGEAETRECIYYNANWELERTNQSGLERCEGEQDKRLHCYASWRNSSGTIELVKKGC |
| | WLDDFNCYDRQECVATKDSPEVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 328 | GRGEAETRECIYYNANWELERTNQSGLERCEGEQDKRLHCYASWRNSSGTIELVKKGC |
| | WLDDFNCYDRQECVATKENPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 329 | RGEAETRECIYYNANWELERTNQSGLERCEGEQDKRRHCFATWKNISGSIEIVKQGCWL |
| | DDFNCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 330 | RGEAETRECIYYNANWELERTNQSGLERCEGEQDKRLHCYASWRNSSGTIEIVKQGCWL |
| | DDETCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 331 | ILGRAETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 332 | ILGRSETQECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 333 | RGEAETRECIYYNANWELERTNQSGLERCEGEQDKRLHCYASWRNSSGTIELVKKGCWL |
| | DDFNCYDRQECVATKDSPEVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 334 | RGEAETRECIYYNANWELERTNQSGLERCEGEQDKRLHCYASWRNSSGTIELVKKGCWL |
| | DDFNCYDRQECVATKENPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 335 | GEAETRECIYYNANWELERTNQSGLERCEGEQDKRRHCFATWKNISGSIEIVKQGCWLD |
| | DFNCYDRTDCVEKKDSPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 336 | GEAETRECIYYNANWELERTNQSGLERCEGEQDKRLHCYASWRNSSGTIEIVKQGCWLD |
| | DETCYDRTDCVEKKDSPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 337 | LGRAETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 338 | LGRSETQECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHTCPP |
| | CPAPELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 339 | GEAETRECIYYNANWELERTNQSGLERCEGEQDKRLHCYASWRNSSGTIELVKKGCWLD |
| | DFNCYDRQECVATKDSPEVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 340 | GEAETRECIYYNANWELERTNQSGLERCEGEQDKRLHCYASWRNSSGTIELVKKGCWLD |
| | DFNCYDRQECVATKENPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 341 | EAETRECIYYNANWELERTNQSGLERCEGEQDKRRHCFATWKNISGSIEIVKQGCWLDDF |
| | NCYDRTDCVEKKDSPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 342 | EAETRECIYYNANWELERTNQSGLERCEGEQDKRLHCYASWRNSSGTIEIVKQGCWLDD |
| | ETCYDRTDCVEKKDSPQVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 343 | GRAETRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 344 | GRSETQECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTSGGGDKTHTCPPC |
| | PAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 345 | EAETRECIYYNANWELERTNQSGLERCEGEQDKRLHCYASWRNSSGTIELVKKGCWLDD |
| | FNCYDRQECVATKDSPEVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 346 | EAETRECIYYNANWELERTNQSGLERCEGEQDKRLHCYASWRNSSGTIELVKKGCWLDD |
| | FNCYDRQECVATKENPQVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 347 | AETRECIYYNANWELERTNQSGLERCEGEQDKRRHCFATWKNISGSIEIVKQGCWLDDFN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 348 | AETRECIYYNANWELERTNQSGLERCEGEQDKRLHCYASWRNSSGTIEIVKQGCWLDDE |
| | TCYDRTDCVEKKDSPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 349 | RAETRECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 350 | RSETQECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTSGGGDKTHTCPPCP |
| | APELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 351 | AETRECIYYNANWELERTNQSGLERCEGEQDKRLHCYASWRNSSGTIELVKKGCWLDDF |
| | NCYDRQECVATKDSPEVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 352 | AETRECIYYNANWELERTNQSGLERCEGEQDKRLHCYASWRNSSGTIELVKKGCWLDDF |
| | NCYDRQECVATKENPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 353 | ETRECIYYNANWELERTNQSGLERCEGEQDKRRHCFATWKNISGSIEIVKQGCWLDDEN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 354 | ETRECIYYNANWELERTNQSGLERCEGEQDKRLHCYASWRNSSGTIEIVKQGCWLDDET |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 355 | AETRECIYYNANWELERTNQSGLERCEGEQDKRLHCYASWRNSSGTIEIVKQGCWLDDF |
| | NCYDRTDCVEKKDSPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 356 | SETQECIYYNANWELERTNQSGLERCEGEQDKRLHCYASWRNSSGTIEIVKQGCWLDDF |
| | NCYDRTDCVEKKDSPQVYFCCCEGNMCNEKFSYFPEMEVTQPTSGGGDKTHTCPPCPA |
| | PELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| 357 | ETRECIYYNANWELERTNQSGLERCEGEQDKRLHCYASWRNSSGTIELVKKGCWLDDFN |
| | CYDRQECVATKDSPEVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 358 | ETRECIYYNANWELERTNQSGLERCEGEQDKRLHCYASWRNSSGTIELVKKGCWLDDFN |
| | CYDRQECVATKENPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 359 | TRECIYYNANWELERTNQSGLERCEGEQDKRRHCFATWKNISGSIEIVKQGCWLDDFNC |
| | YDRTDCVEKKDSPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 360 | TRECIYYNANWELERTNQSGLERCEGEQDKRLHCYASWRNSSGTIEIVKQGCWLDDETC |
| | YDRTDCVEKKDSPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 361 | QECIYYNANWELERTNQSGLERCEGEQDKRLHCYASWRNSSGTIEIVKQGCWLDDENCY |
| | DRTDCVEKKDSPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 362 | ETRECIYYNANWELERTNQSGLERCEGEQDKRLHCYASWRNSSGTIEIVKQGCWLDDFN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 363 | ETQECIYYNANWELERTNQSGLERCEGEQDKRLHCYASWRNSSGTIEIVKQGCWLDDFN |
| | CYDRTDCVEKKDSPQVYFCCCEGNMCNEKFSYFPEMEVTQPTSGGGDKTHTCPPCPAP |
| | ELLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 364 | TRECIYYNANWELERTNQSGLERCEGEQDKRLHCYASWRNSSGTIELVKKGCWLDDFNC |
| | YDRQECVATKDSPEVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 365 | TRECIYYNANWELERTNQSGLERCEGEQDKRLHCYASWRNSSGTIELVKKGCWLDDFNC |
| | YDRQECVATKENPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 366 | RECIYYNANWELERTNQSGLERCEGEQDKRRHCFATWKNISGSIEIVKQGCWLDDENCY |
| | DRTDCVEKKDSPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 367 | RECIYYNANWELERTNQSGLERCEGEQDKRLHCYASWRNSSGTIEIVKQGCWLDDETCY |
| | DRTDCVEKKDSPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 368 | TRECIYYNANWELERTNQSGLERCEGEQDKRLHCYASWRNSSGTIEIVKQGCWLDDFNC |
| | YDRTDCVEKKDSPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 369 | TQECIYYNANWELERTNQSGLERCEGEQDKRLHCYASWRNSSGTIEIVKQGCWLDDFNC |
| | YDRTDCVEKKDSPQVYFCCCEGNMCNEKFSYFPEMEVTQPTSGGGDKTHTCPPCPAPE |
| | LLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 370 | RECIYYNANWELERTNQSGLERCEGEQDKRLHCYASWRNSSGTIELVKKGCWLDDENCY |
| | DRQECVATKDSPEVYFCCCEGNMCNEKFSYFPEMEVTQPTSGGGDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 371 | RECIYYNANWELERTNQSGLERCEGEQDKRLHCYASWRNSSGTIELVKKGCWLDDENCY |
| | DRQECVATKENPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 372 | RECIYYNANWELERTNQSGLERCEGEQDKRLHCYASWRNSSGTIEIVKQGCWLDDENCY |
| | DRTDCVEKKDSPQVYFCCCEGNMCNEKFSYFPEMEVTQPTSGGGDKTHTCPPCPAPEL |
| | LGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 373 | GRGEAETRECIYYNANWELERTNQSGLERCEGEQRKRLHCYASWRNSSGTIEIVKQGCW |
| | LDDFNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |

Furthermore, in some embodiments, a polypeptide described herein (e.g., an ActRII chimera-Fc fusion protein) has a serum half-life of at least 7 days in humans. The polypeptide including an extracellular ActRII chimera may bind to activin A with a K_{D} of 10 pM or higher. In some embodiments, the polypeptide including an extracellular ActRII chimera binds to activin A, activin B, and/or myostatin and exhibits reduced (e.g., weak) binding to BMP9 (e.g., compared to wild-type extracellular ActRIIB). In some embodiments, the polypeptide including an extracellular ActRII chimera that has reduced or weak binding to BMP9 has the sequence TEEN (SEQ ID NO: 374) or TKEN (SEQ ID NO: 375) at positions X₃, X₄, X₅, and X₆. In some embodiments, the polypeptide including an extracellular ActRII chimera that has reduced or weak binding to BMP9 has the sequence KKDS (SEQ ID NO: 376) or TKDS (SEQ ID NO: 377) at positions X₃, X₄, X₅, and X₆. In some embodiments, the polypeptide including an extracellular ActRII chimera does not substantially bind to human BMP9.

In some embodiments, the polypeptide including an extracellular ActRII chimera may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 30 pM). In some embodiments, the polypeptide including an extracellular ActRII chimera may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 5 pM). The polypeptide including an extracellular ActRII chimera may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

### Fc Domains

In some embodiments, a polypeptide described herein may include an extracellular ActRII variant (e.g., an extracellular ActRIIA variant, an extracellular ActRIIB variant, or an extracellular ActRII chimera) fused to an Fc domain monomer of an immunoglobulin or a fragment of an Fc domain to increase the serum half-life of the polypeptide. A polypeptide including an extracellular ActRII variant fused to an Fc domain monomer may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. A wild-type Fc domain forms the minimum structure that binds to an Fc receptor, e.g., FcγRI, FcγRIIa, FcγRIIb, FcγRIIIa, FcγRIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor. In some embodiments, an Fc domain is from an IgG1 antibody and includes amino acid substitutions L234A, L235A, and G237A. In some embodiments, an Fc domain is from an IgG1 antibody and includes amino acid substitutions D265A, K322A, and N434A. The aforementioned amino acid positions are defined according to Kabat (Sequences of Proteins of Immunological Interest, 5th Ed. Public Health Service, National Institutes of Health, Bethesda, MD. (1991)). The Kabat numbering of amino acid residues may be determined for a given antibody by alignment at regions of homology of the sequence of the antibody with a “standard” Kabat numbered sequence. Furthermore, in some embodiments, an Fc domain does not induce any immune system-related response. For example, the Fc domain in a dimer of a polypeptide including an extracellular ActRII variant fused to an Fc domain monomer may be modified to reduce the interaction or binding between the Fc domain and an Fcγ receptor. The sequence of an Fc domain monomer that may be fused to an extracellular ActRII variant (e.g., an extracellular ActRIIA variant, an extracellular ActRIIB variant, or an extracellular ActRII chimera) is shown below (SEQ ID NO: 97):

THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPVPIEKTISKAKGQPREPQVYTLPPSREEMTKNQVSLTCLVKGF
YPSDIAVEWESNGQPENNYKTTPPVLDSDGPFFLYSKLTVDKSRWQQGNV
FSCSVMHEALHNHYTQKSLSLSPGK

The sequence of a wild-type Fc domain monomer that may be fused to an extracellular ActRII variant (e.g., an extracellular ActRIIA variant, an extracellular ActRIIB variant, or an extracellular ActRII chimera) is shown below in SEQ ID NO: 150:

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

In some embodiments, the Fc domain monomer fused to an extracellular ActRII variant lacks a terminal lysine. An exemplary sequence for a wild-type Fc domain monomer lacking the terminal lysine is provided below (SEQ ID NO: 155):

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG.

In some embodiments, an Fc domain is from an IgG1 antibody and includes amino acid substitutions L12A, L13A, and G15A, relative to the sequence of SEQ ID NO: 97. In some embodiments, an Fc domain is from an IgG1 antibody and includes amino acid substitutions D43A, K100A, and N212A, relative to the sequence of SEQ ID NO: 97. In some embodiments, the terminal lysine is absent from the Fc domain monomer having the sequence of SEQ ID NO: 97 or SEQ ID NO: 150. In some embodiments, an extracellular ActRII variant described herein (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)) may be fused to the N- or C-terminus of an Fc domain monomer (e.g., SEQ ID NO: 97, SEQ ID NO: 150, or SEQ ID NO: 155) through conventional genetic or chemical means, e.g., chemical conjugation. If desired, a linker (e.g., a spacer) can be inserted between the extracellular ActRII variant and the Fc domain monomer. The Fc domain monomer can be fused to the N- or C-terminus (e.g., C-terminus) of the extracellular ActRII variant. The Fc domain monomer can be of immunoglobulin antibody isotype IgG, IgE, IgM, IgA, or IgD. Additionally, the Fc domain monomer can be an IgG subtype (e.g., IgG1, IgG2a, IgG2b, IgG3, or IgG4). In some embodiments, the Fc domain monomer is an IgG1 Fc domain monomer (e.g., a human IgG1 Fc domain monomer).

In some embodiments, the Fc domain contains one or more amino acid substitutions that reduce or inhibit Fc domain dimerization. In some embodiments, the Fc domain contains a hinge domain. The Fc domain can be of immunoglobulin antibody isotype IgG, IgE, IgM, IgA, or IgD. Additionally, the Fc domain can be an IgG subtype (e.g., IgG1, IgG2a, IgG2b, IgG3, or IgG4). The Fc domain can also be a non-naturally occurring Fc domain, e.g., a recombinant Fc domain.

Methods of engineering Fc domains that have reduced dimerization are known in the art. In some embodiments, one or more amino acids with large side chains (e.g., tyrosine or tryptophan) may be introduced to the C_{H}3-C_{H}3 dimer interface to hinder dimer formation due to steric clash. In other embodiments, one or more amino acids with small side chains (e.g., alanine, valine, or threonine) may be introduced to the C_{H}3-C_{H}3 dimer interface to remove favorable interactions. Methods of introducing amino acids with large or small side chains in the C_{H}3 domain are described in, e.g., Ying et al. (J Biol Chem. 287:19399-19408, 2012), U.S. Patent Publication No. 2006/0074225, U.S. Pat. Nos. 8,216,805 and 5,731,168, Ridgway et al. (Protein Eng. 9:617-612, 1996), Atwell et al. (J Mol Biol. 270:26-35, 1997), and Merchant et al. (Nat Biotechnol. 16:677-681, 1998), all of which are incorporated herein by reference in their entireties.

In yet other embodiments, one or more amino acid residues in the C_{H}3 domain that make up the C_{H}3-C_{H}3 interface between two Fc domains are replaced with positively charged amino acid residues (e.g., lysine, arginine, or histidine) or negatively charged amino acid residues (e.g., aspartic acid or glutamic acid) such that the interaction becomes electrostatically unfavorable depending on the specific charged amino acids introduced. Methods of introducing charged amino acids in the C_{H}3 domain to disfavor or prevent dimer formation are described in, e.g., Ying et al. (J Biol Chem. 287:19399-19408, 2012), U.S. Patent Publication Nos. 2006/0074225, 2012/0244578, and 2014/0024111, all of which are incorporated herein by reference in their entireties.

In some embodiments, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y4071, K409E, K409D, K409T, and K409I, relative to the sequence of human IgG1. In some embodiments, the terminal lysine is absent from the Fc domain amino acid sequence. In one particular embodiment, an Fc domain includes the amino acid substitution T366W, relative to the sequence of human IgG1.

### Linkers

A polypeptide described herein may include an extracellular ActRII variant (e.g., an extracellular ActRIIA variant having a sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)) fused to an Fc domain monomer by way of a linker. In some embodiments, the Fc domain monomer increases stability of the polypeptide. In the present invention, a linker between an Fc domain monomer (e.g., SEQ ID NO: 97, SEQ ID NO: 150, or SEQ ID NO: 155) and an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)), can be an amino acid spacer including 1-200 amino acids. Suitable peptide spacers are known in the art, and include, for example, peptide linkers containing flexible amino acid residues such as glycine, alanine, and serine. In some embodiments, a spacer can contain motifs, e.g., multiple or repeating motifs, of GA, GS, GG, GGA, GGS, GGG, GGGA (SEQ ID NO: 98), GGGS (SEQ ID NO: 99), GGGG (SEQ ID NO: 100), GGGGA (SEQ ID NO: 101), GGGGS (SEQ ID NO: 102), GGGGG (SEQ ID NO: 103), GGAG (SEQ ID NO: 104), GGSG (SEQ ID NO: 105), AGGG (SEQ ID NO: 106), or SGGG (SEQ ID NO: 107). In some embodiments, a spacer can contain 2 to 12 amino acids including motifs of GA or GS, e.g., GA, GS, GAGA (SEQ ID NO: 108), GSGS (SEQ ID NO: 109), GAGAGA (SEQ ID NO: 110), GSGSGS (SEQ ID NO: 111), GAGAGAGA (SEQ ID NO: 112), GSGSGSGS (SEQ ID NO: 113), GAGAGAGAGA (SEQ ID NO: 114), GSGSGSGSGS (SEQ ID NO: 115), GAGAGAGAGAGA (SEQ ID NO: 116), and GSGSGSGSGSGS (SEQ ID NO: 117). In some embodiments, a spacer can contain 3 to 12 amino acids including motifs of GGA or GGS, e.g., GGA, GGS, GGAGGA (SEQ ID NO: 118), GGSGGS (SEQ ID NO: 119), GGAGGAGGA (SEQ ID NO: 120), GGSGGSGGS (SEQ ID NO: 121), GGAGGAGGAGGA (SEQ ID NO: 122), and GGSGGSGGSGGS (SEQ ID NO: 123). In yet some embodiments, a spacer can contain 4 to 12 amino acids including motifs of GGAG (SEQ ID NO: 104), GGSG (SEQ ID NO: 105), e.g., GGAG (SEQ ID NO: 104), GGSG (SEQ ID NO: 105), GGAGGGAG (SEQ ID NO: 124), GGSGGGSG (SEQ ID NO: 125), GGAGGGAGGGAG (SEQ ID NO: 126), and GGSGGGSGGGSG (SEQ ID NO: 127). In some embodiments, a spacer can contain motifs of GGGGA (SEQ ID NO: 101) or GGGGS (SEQ ID NO: 102), e.g., GGGGAGGGGAGGGGA (SEQ ID NO: 128) and GGGGSGGGGSGGGGS (SEQ ID NO: 129). In some embodiments, an amino acid spacer between an Fc domain monomer and an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)) may be GGG, GGGA (SEQ ID NO: 98), GGGG (SEQ ID NO: 100), GGGAG (SEQ ID NO: 130), GGGAGG (SEQ ID NO: 131), or GGGAGGG (SEQ ID NO: 132).

In some embodiments, a spacer can also contain amino acids other than glycine, alanine, and serine, e.g., AAAL (SEQ ID NO: 133), AAAK (SEQ ID NO: 134), AAAR (SEQ ID NO: 135), EGKSSGSGSESKST (SEQ ID NO: 136), GSAGSAAGSGEF (SEQ ID NO: 137), AEAAAKEAAAKA (SEQ ID NO: 138), KESGSVSSEQLAQFRSLD (SEQ ID NO: 139), GENLYFQSGG (SEQ ID NO: 140), SACYCELS (SEQ ID NO: 141), RSIAT (SEQ ID NO: 142), RPACKIPNDLKQKVMNH (SEQ ID NO: 143), GGSAGGSGSGSSGGSSGASGTGTAGGTGSGSGTGSG (SEQ ID NO: 144), AAANSSIDLISVPVDSR (SEQ ID NO: 145), or GGSGGGSEGGGSEGGGSEGGGSEGGGSEGGGSGGGS (SEQ ID NO: 146). In some embodiments, a spacer can contain motifs, e.g., multiple or repeating motifs, of EAAAK (SEQ ID NO: 147). In some embodiments, a spacer can contain motifs, e.g., multiple or repeating motifs, of proline-rich sequences such as (XP)n (SEQ ID NO: 378), in which X may be any amino acid (e.g., A, K, or E) and n is from 1-5, and PAPAP (SEQ ID NO: 148).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, the linker between an Fc domain monomer (e.g., SEQ ID NO: 97, SEQ ID NO: 150, or SEQ ID NO: 155) and an extracellular ActRII variant described herein (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)), is an amino acid spacer having the sequence GGG. For example, a polypeptide described herein can contain an extracellular ActRIIA variant (e.g., any one of SEQ ID NOs: 6-72) fused to an Fc domain monomer (e.g., SEQ ID NO: 155) by a GGG linker. An exemplary polypeptide containing an ActRIIA variant of SEQ ID NO: 69, a GGG linker, and an Fc domain monomer lacking a terminal lysine (SEQ ID NO: 155) is provided below (SEQ ID NO: 156):

GAILGRSETQECLFYNANWELERTNQTGVERCEGEKDKRLHCYATWRNIS
GSIEIVKKGCWLDDFNCYDRTDCVETEENPQVYFCCCEGNMCNEKFSYFP
EMEVTQPTSGGGDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNHYTQKSLSLSPG

In another example, a polypeptide described herein can contain an extracellular ActRIIB variant (e.g., any one of SEQ ID NOs: 158-171) fused to an Fc domain monomer (e.g., SEQ ID NO: 150 or SEQ ID NO: 155) by a GGG linker. An exemplary polypeptide containing an ActRIIB variant of SEQ ID NO: 171, a GGG linker, and an Fc domain monomer (SEQ ID NO: 150) is provided below (SEQ ID NO: 172):

GRGEAETRECLYYNANWELERTNQSGVERCEGEKDKRLHCYASWRNSSG
SLEIVKKGCWLDDFNCYDRDTCVATKENPQVYFCCCEGNMCNERFTHLP
EAGGPEVTYEPPPTAPTGGGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK

In another example, a polypeptide described herein can contain an extracellular ActRII chimera (e.g., any one of SEQ ID NOs: 195-216) fused to an Fc domain monomer (e.g., SEQ ID NO: 150 or SEQ ID NO: 155) by a GGG linker. An exemplary polypeptide containing an ActRII chimera of SEQ ID NO: 214, a GGG linker, and an Fc domain monomer (SEQ ID NO: 150) is provided below (SEQ ID NO: 326):

GAILGRSETQECIYYNANWELERTNQSGLERCEGEQDKRLHCYASWRNSS
GTIEIVKQGCWLDDFNCYDRTDCVEKKDSPQVYFCCCEGNMCNEKFSYFP
EMEVTQPTSGGGDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNHYTQKSLSLSPGK

The C-terminal Lys339 of the polypeptide of SEQ ID NO: 326 (the C-terminal Lys in the Fc region of SEQ ID NO: 326) and the C-terminal Lys345 of the polypeptide of SEQ ID NO: 172 (the C-terminal Lys in the Fc region of SEQ ID NO: 172) may or may not be present, without affecting the structure or stability of the polypeptide. The disclosure specifically contemplates SEQ ID NO: 326 that does not include the C-terminal Lys corresponding to Lys339 and SEQ ID NO: 172 that does not include the C-terminal Lys corresponding to Lys345. The polypeptide of SEQ ID NO: 326 may be expressed including a C-terminal Lys339 which then may be proteolytically cleaved upon expression of the polypeptide, and the polypeptide of SEQ ID NO: 172 may be expressed including a C-terminal Lys345 which then may be proteolytically cleaved upon expression of the polypeptide (e.g., the polypeptides of SEQ ID NO: 326 and SEQ ID NO: 172 are expressed using nucleic acid constructs encoding the polypeptide including a C-terminal lysine residue). The polypeptides of SEQ ID NO: 326 and SEQ ID NO: 172 may also be expressed without including the C-terminal Lys339 and the C-terminal Lys345, respectively.

### Vectors, Host Cells, and Protein Production

The polypeptides described herein can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of a polypeptide described herein may be prepared by a variety of methods known in the art. These methods include, but are not limited to, oligonucleotide-mediated (or site-directed) mutagenesis and PCR mutagenesis. A nucleic acid molecule encoding a polypeptide described herein may be obtained using standard techniques, e.g., gene synthesis. Alternatively, a nucleic acid molecule encoding a wild-type extracellular ActRIIA or ActRIIB may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding a polypeptide described herein may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7O3O, and HsS78Bst cells. In some embodiments, E. coli cells may also be used as host cells for the invention. Examples of E. coli strains include, but are not limited to, E. coli 294 (ATCC® 31,446), E. coli λ 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the polypeptides described herein may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide described herein may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the polypeptides described herein can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide described herein. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions and Preparations

The invention features pharmaceutical compositions that include the polypeptides described herein (e.g., a polypeptide including an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)). In some embodiments, a pharmaceutical composition includes a polypeptide including an extracellular ActRII variant (e.g., an extracellular ActRIIA variant having the sequence of any one of SEQ ID NOs: 1-70 (e.g., SEQ ID NOs: 6-70) or an extracellular ActRII chimera having the sequence of any one of SEQ ID NOs: 174-216 (e.g., 195-216)) with a C-terminal extension (e.g., 1, 2, 3, 4, 5, 6 or more additional amino acids) as the therapeutic protein. In some embodiments, a pharmaceutical composition includes a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)) fused to an Fc domain monomer as the therapeutic protein. In some embodiments, a pharmaceutical composition including a polypeptide described herein may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, a pharmaceutical composition includes a nucleic acid molecule (DNA or RNA, e.g., mRNA) encoding a polypeptide described herein, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, arginine, and lysine, and carbohydrates such as glucose, mannose, sucrose, and sorbitol. Pharmaceutical compositions described herein can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical compositions may be prepared in microcapsules, such as hydroxylmethylcellulose or gelatin-microcapsule and poly-(methylmethacrylate) microcapsule. The pharmaceutical compositions may also be prepared in other drug delivery systems such as liposomes, albumin microspheres, microemulsions, nanoparticles, and nanocapsules. Such techniques are described in Remington: The Science and Practice of Pharmacy 22^{nd} edition (2012). The pharmaceutical compositions to be used for in vivo administration must be sterile. This is readily accomplished by filtration through sterile filtration membranes.

The pharmaceutical compositions may also be prepared as a sustained-release formulation. Suitable examples of sustained-release preparations include semipermeable matrices of solid hydrophobic polymers containing the polypeptides described herein. Examples of sustained release matrices include polyesters, hydrogels, polylactides, copolymers of L-glutamic acid and γ ethyl-L-glutamate, non-degradable ethylene-vinyl acetate, degradable lactic acid-glycolic acid copolymers such as LUPRON DEPOT™, and poly-D-(-)-3-hydroxybutyric acid. Some sustained-release formulations enable release of molecules over a few months, e.g., one to six months, while other formulations release pharmaceutical compositions for shorter time periods, e.g., days to weeks.

The pharmaceutical composition may be formed in a unit dose form as needed. The amount of active component, e.g., a polypeptide described herein, included in the pharmaceutical preparations is such that a suitable dose within the designated range is provided (e.g., a dose within the range of 0.01-100 mg/kg of body weight).

The pharmaceutical composition for gene therapy can be in an acceptable diluent or can include a slow-release matrix in which the gene delivery vehicle is imbedded. If hydrodynamic injection is used as the delivery method, the pharmaceutical composition containing a nucleic acid molecule encoding a polypeptide described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### Routes, Dosage, and Administration

Pharmaceutical compositions that include the polypeptides described herein as the therapeutic proteins may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide described herein or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides described herein may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide described herein may be administered using hydrodynamic injection.

The dosage of the pharmaceutical compositions depends on factors including the route of administration, the disease to be treated, and physical characteristics, e.g., age, weight, general health, of the subject. A pharmaceutical composition may include a dosage of a polypeptide described herein ranging from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 0.1-100 mg/kg, e.g., 0.5-50 mg/kg. Pharmaceutical compositions that include a polypeptide described herein may be administered to a subject in need thereof, for example, one or more times (e.g., 1-10 times or more) daily, weekly, biweekly, every four weeks, monthly, every eight weeks, bimonthly, every 12 weeks, quarterly, every sixteen weeks biannually, annually, or as medically necessary. In some embodiments, pharmaceutical compositions that include a polypeptide described herein may be administered to a subject in need thereof weekly, biweekly, every four weeks, monthly, every eight weeks, bimonthly, every twelve weeks, quarterly, or every sixteen weeks. Dosages may be provided in either a single or multiple dosage regimens. The timing between administrations may increase as the medical condition improves or decrease as the health of the patient declines.

### Methods of Treatment

The ActRII variants described herein have improved properties compared to endogenous ActRIIA and ActRIIB. For example, the ActRIIA variants generated by introducing residues from ActRIIB into ActRIIA retain the beneficial properties of ActRIIA, such as low binding affinity to BMP9 and longer serum half-life as an Fc fusion protein, and gain some of the beneficial properties of ActRIIB, such as increased binding to activins A and B. The ActRIIB variants generated by introducing residues from ActRIIA into ActRIIB may retain the beneficial properties of ActRIIB, such high binding affinity to activins A and B, and gain some of the beneficial properties of ActRIIA, such as reduced binding affinity to BMP9 and longer serum half-life as an Fc fusion protein. In addition, the ActRII chimeras generated by combining extracellular portions of ActRIIA and ActRIIB may possess beneficial properties of both ActRIIB (e.g., strong binding affinity to activins A and B) and ActRIIA (e.g., reduced binding affinity to BMP9 and/or longer serum half-life as an Fc fusion protein (e.g., compared to ActRIIB-Fc)). These ActRIIA variant, ActRIIB variant, and ActRII chimera properties make for a useful therapeutic that can compete with endogenous activin receptors for ligand binding. As the ActRIIA variants, ActRIIB variants, and ActRII chimeras contain the extracellular portion of the receptor, they will be soluble and able to bind to and sequester ligands (e.g., activins A and B, myostatin, GDF11) without activating intracellular signaling pathways. Based on the discovery that administration of a polypeptide containing an ActRIIB variant described herein decreased serum proteins involved in inflammation and fibrosis, increased anti-inflammatory cytokines, and decreased NT pro-BNP in human subjects and attenuated cardiac remodeling and fibrosis in a mouse model of TAC, and that administration of a polypeptide containing an ActRIIA variant described herein reduced NT-proBNP levels in human subjects with lower-risk MDS, anemia, and elevated NT-proBNP levels (2450 pg/mL) and attenuated cardiac remodeling in a mouse mode of TAC, polypeptides containing the ActRII variants described herein (e.g., ActRIIA variants, ActRIIB variants, or ActRII chimeras) can be used to treat a cardiovascular disease or to reduce cardiovascular risk (e.g., prevent, delay, or reduce the likelihood of developing a cardiovascular disease).

The polypeptides described herein (e.g., a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)), e.g., an effective amount of an ActRIIA variant, ActRIIB variant, or ActRII chimera) can be used to treat a subject having a cardiovascular disease or to prevent, delay the development of, slow the progression of, or reverse the progression of a cardiovascular disease in a subject having or at risk of developing such a disease. A subject at risk of developing a cardiovascular disease may have one or more risk factors for development of a cardiovascular disease, such as advanced age, tobacco use, physical inactivity, excessive alcohol consumption, unhealthy diet, obesity, genetic predisposition or family history of cardiovascular disease, elevated blood pressure (hypertension), elevated blood sugar (diabetes mellitus), elevated blood cholesterol (hyperlipidemia), undiagnosed celiac disease, poverty, low educational status, or exposure to air pollution.

In some embodiments, the cardiovascular disease that can be treated according to the methods described herein is a calcification disease, such as Monckeberg's vascular calcification disease, vascular calcification, or valvular calcification (e.g., cardiac valvular calcification). In some embodiments, the cardiovascular disease is cardiomyopathy, such as dilated cardiomyopathy, hypertrophic cardiomyopathy, restrictive cardiomyopathy, arrhythmogenic right ventricular dysplasia, unclassified cardiomyopathy, pediatric cardiomyopathy, or peripartum cardiomyopathy. In some embodiments, the cardiovascular disease is heart failure, such as left-sided (left ventricular) heart failure or right-sided (right ventricular) heart failure (i.e., heart failure associated with weakening of the left ventricle or right ventricle, respectively). There are two types of left-sided heart failure: systolic failure, which is also known as heart failure with reduced ejection fraction (HFrEF) (left ventricular ejection fraction (LVEF)≥40%), and diastolic failure, which is also known as heart failure with preserved ejection fraction (HFpEF) (LVEF≥50%). In some embodiments, the cardiovascular disease is backward heart failure, forward heart failure, high-output heart failure, low-output heart failure, compensated heart failure, or decompensated heart failure. In some embodiments, the heart failure is chronic (e.g., chronic HFrEF or chronic HFpEF). In some embodiments, the heart failure is acute (e.g., acute HFrEF, acute HFpEF, or acute decompensated heart failure). In some embodiments, the cardiovascular disease is heart valve disease, such as aortic stenosis (e.g., renal artery stenosis), mitral valve insufficiency, mitral valve prolapse, or rheumatic heart disease. In some embodiments, the cardiovascular disease is ventricular dysfunction (e.g., left ventricular dysfunction or right ventricular dysfunction). The polypeptides described herein can help with heart valve recovery in a subject having a heart valve disease or provide temporary protection before valve replacement. In some embodiments, the cardiovascular disease is vasculitis or deep vein thrombosis. In some embodiments, the cardiovascular disease is an arrhythmia, such as atrial flutter or atrial fibrillation (e.g., symptomatic atrial flutter or atrial fibrillation or paroxysmal atrial flutter or atrial fibrillation), ventricular arrythmia (e.g., recurrent ventricular fibrillation (VF) or recurrent hemodynamically unstable ventricular tachycardia (VT)), cardiac arrhythmia associated with myocardial ischemia or myocardial infarction, or arrhythmia associated with cerebral hemorrhage.

In some embodiments, the compositions and methods described herein can be used to treat a subject having or at risk of developing hyperlipidemia or hyperlipoproteinemia, including congenital or acquired hyperlipidemia or hyperlipoproteinemia. In some embodiments, the congenital hyperlipidemia or hyperlipoproteinemia is familial combined hyperlipidemia (FCHL), primary hyperlipoproteinemia, hyperapobetalipoproteinemia, Frederickson type III hyperlipidemia (familial dysbetalipoprotenemia), Frederickson type IV hyperlipidemia (familial hypertriglyceridemia), Frederickson type V hyperlipidemia (endogenous hypertriglyceridemia), or small dense LDL syndrome (LDL phenotype B). In some embodiments, the acquired hyperlipidemia or hyperlipoproteinemia is associated with diabetes mellitus, hyperlipidemic diet and/or sedentary lifestyle, obesity, metabolic syndrome, intrinsic or secondary liver disease, primary biliary cirrhosis or other bile stasis disorders, alcoholism, pancreatitis, nephrotic syndrome, end-stage renal disease, hypothyroidism, or iatrogenesis due to administration of thiazides, beta-blockers, retinoids, highly active antiretroviral agents, estrogen, progestins, or glucocorticoids. In some embodiments, the cardiovascular disease is a disease, disorder, or syndrome associated with defects in lipid absorption or metabolism, such as sitosterolemia, cerebrotendinous xanthomatosis, or familial hypobetalipoproteinemia. In some embodiments, the methods described herein can be used to treat a disease, disorder, or syndrome associated with hyperlipidemia, such as an aortic aneurysm (e.g., an abdominal aortic aneurysm or thoracic aortic aneurysm) pseudoaneurysm, intramural hematoma, aortic stenosis, vascular calcification, xanthoma, such as xanthoma affecting tendons or scleral and cutaneous xanthomas, xanthelasma, or hepatosteatosis. In some embodiments, the compositions and methods described herein can be used to treat a subject having mixed dyslipidemia, hypertriglyceridemia, or hyperlipidemia in an HIV positive subject. In some embodiments, the compositions and methods described herein can be used to reduce or prevent aneurysm formation.

In some embodiments, the subject is diagnosed as having or at risk of developing the cardiovascular disease or condition prior to treatment with a polypeptide described herein. In some embodiments, the method includes the step of diagnosing the subject as having or as at risk of developing the cardiovascular disease or condition before initiating treatment with a polypeptide described herein.

The polypeptides described herein (e.g., a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)), e.g., an effective amount of an ActRIIA variant, ActRIIB variant, or ActRII chimera) can be used to treat a subject with HFpEF (LVEF≥50%) or HFrEF (LVEF≤40%) (e.g., chronic HFpEF or chronic HFrEF) with elevated N-terminal pro b-type natriuretic peptide (NT-proBNP) (>500 pg/mL). In some embodiments, the subject has HFpEF (LVEF≥50%) or HFrEF (LVEF≤40%) (e.g., chronic HFpEF or chronic HFrEF) with elevated NT-proBNP (>500 pg/mL) despite an appropriate and stable heart failure-directed medical regimen (stable therapy is defined as no change in dose/regimen for at least 30 days prior to Baseline, Baseline is defined as the last available, valid, non-missing assessment on or prior to the first dose of a polypeptide described herein). In some embodiments, the subject has HFpEF (e.g., chronic HFpEF) with defined ≥Grade 2 diastolic dysfunction. In some embodiments, the subject has HFpEF (e.g., chronic HFpEF) with defined body mass index (BMI) of ≥30 and ≤35 kg/m2. In some embodiments, the subject has stable New York Heart Association (NYHA) Functional Class II to IV symptoms (e.g., NYHA Functional Class II symptoms, NYHA Functional Class III symptoms, or NYHA Functional Class IV symptoms) for at least 30 days prior to treatment initiation.

In some embodiments, a subject with HFpEF or HFrEF (e.g., chronic HFpEF or chronic HFrEF with elevated NT-proBNP (>500 pg/mL)) treated according to the methods described herein is administered a polypeptide described herein in combination with one or more heart failure (HF) background therapies. The HF background therapy may be an approved HF therapy, such as a sodium-glucose cotransporter-2 inhibitor, a vasodilator, a diuretic, a neurohormonal antagonist (e.g., a beta blocker, an angiotensin receptor blocker, such as an angiotensin II receptor blocker, an angiotensin converting enzyme inhibitor, or a mineralocorticoid receptor antagonist), an aldosterone antagonist, a positive inotrope, a soluble guanylate cyclase stimulator, a nitrate derivative, or an angiotensin receptor neprilysin inhibitor (e.g., sacubitril-valsartan). In some embodiments, the subject has been taking the one or more HF background therapies prior to treatment with a polypeptide described herein (e.g., taking the one or more HF background therapies for 1 week, 1 month, 2 months, 3 months, 4 months, 5 months, 6 months, 1 year, 2 years, or longer prior to treatment initiation with a polypeptide described herein). In some embodiments, the subject has been taking the one or more HF background therapies for at least 30 days prior to treatment initiation with a polypeptide described herein. In some embodiments, the subject has been on stable HF background therapy for at least 30 days prior to Baseline (stable therapy is defined as no change in dose/regimen of HF background therapy). In some embodiments, the HF background therapy is administered as indicated on the label (e.g., for the treatment of subjects with HF, e.g., HFpEF or HFrEF).

In some embodiments, the compositions and methods described herein reduce fibrosis, reduce inflammation (e.g., vascular inflammation or cardiac inflammation), reduce cardiac remodeling, improve cardiac function, or improve ventricular function (e.g., right or left ventricular function) in a subject having or at risk of developing a cardiovascular disease (e.g., compared to measurements prior to treatment or compared to an untreated subject having the same disease or condition). In some embodiments, the compositions and methods described herein slow or inhibit progression of the cardiovascular disease, delay the development or onset of the cardiovascular disease, or prevent the cardiovascular disease (e.g., when administered to a subject before the development of the cardiovascular disease, such as to a subject having multiple risk factors for the development of the cardiovascular disease or a subject showing initial signs or symptoms of the cardiovascular disease who has not yet been diagnosed as having the cardiovascular disease). In some embodiments, the compositions and methods described herein reverse the progression of the cardiovascular disease (e.g., reduce the severity of the disease, such as from severe disease to moderate disease or from moderate disease to mild disease, e.g., a reduction in NYHA Functional Class, such as from NYHA Functional Class IV to NYHA Functional Class III, from NYHA Functional Class III to NYHA Functional Class II, or from NYHA Functional Class II to NYHA Functional Class I). In some embodiments, the compositions and methods described herein lead to a change (e.g., a decrease) in NT-proBNP from Baseline measurements. In some embodiments, the compositions and methods described herein lead to a change (e.g., an improvement) in cardiopulmonary exercise testing (CPET) parameters (e.g., respiratory gas exchange, heart rate, blood pressure, or other physiological variables assessed by cardiopulmonary exercise test, such as a test using a cycle ergometer or treadmill) from Baseline measurements. In some embodiments, the compositions and methods described herein lead to a change (e.g., an improvement) in echocardiographic (ECHO) parameters (e.g., LVEF or other cardiac function variables measured by echocardiogram) from Baseline measurements. In some embodiments, the compositions and methods described herein reduce or eliminate the need for coronary revascularization (e.g., placement of a stent or coronary bypass surgery), heart valve repair or replacement, implantation of a cardioverter-defibrillator, cardiac resynchronization therapy, a ventricular assist device, or a heart transplant. In some embodiments, the compositions and methods described herein reduce dyspnea (shortness of breath), orthopnea, paroxysmal nocturnal dyspnea, fatigue, and/or fluid retention, or increase exercise tolerance. In some embodiments, the compositions and methods described herein reduce the risk of cardiovascular death, slow the progression of aneurysm formation, limit the progression of vascular calcification, reduce the need for vascular surgery, or reduce secondary cardiovascular events. In some embodiments, the methods described herein improve life expectancy for a subject with a cardiovascular disease. These outcomes can be assessed by comparing measurements obtained after treatment to measurements taken prior to treatment or by comparing measurements obtained after treatment to measurements from subjects with the same cardiovascular disease who were not treated with a composition described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 24 weeks, 25 weeks, 28 weeks, 32 weeks, 36 weeks, 40 weeks, 44 weeks, 48 weeks, 52 weeks, 56 weeks, 60 weeks, 64 weeks, 68 weeks, 72 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

The polypeptides described herein (e.g., a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)), e.g., an effective amount of an ActRIIA variant, ActRIIB variant, or ActRII chimera) can also be used to reduce cardiovascular risk (e.g., reduce the risk of developing a cardiovascular disease, reduce the risk or occurrence of cardiovascular events, slow or inhibit progression of a cardiovascular disease, delay the development or onset of a cardiovascular disease, reduce cardiac strain, improve cardiac function, or reduce the risk of death from a cardiovascular disease, or prevent death from a cardiovascular disease). In some embodiments, the polypeptides described herein are used to reduce cardiovascular risk in a subject having another disease or condition (e.g., a disease or condition other than a cardiovascular disease, such as a disease or condition other than a cardiovascular disease that is known to be associated with increased risk of cardiovascular disease development or death associated with a cardiovascular disease, e.g., in a subject who is being treated with an ActRII variant described herein for a disease or condition other than a cardiovascular disease). For example, the polypeptides described herein may be used to reduce cardiovascular risk in a subject having a myelodysplastic syndrome (MDS). Cardiovascular events represent a major cause of death in patients with lower-risk MDS. Accordingly, in some embodiments, the polypeptides described herein reduce the risk of developing a cardiovascular disease, reduce the risk or occurrence of cardiovascular events, slow or inhibit progression of a cardiovascular disease, delay the development or onset of a cardiovascular disease, reduce cardiac strain, improve cardiac function, reduce the risk of cardiovascular disease-related mortality, or prevent cardiovascular disease-related mortality in a subject having a myelodysplastic syndrome. The subject may be diagnosed as having a myelodysplastic syndrome according to the World Health Organization (WHO) classification or the French American British (FAB) classification. The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del(5q)), myelodysplastic syndrome with excess blasts (MDS-EB; which includes myelodysplastic syndrome with excess blasts—type 1 (MDS-EB-1) and myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk myelodysplastic syndrome as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations, such a subject may have increased erythroid progenitor cells in the bone marrow relative to a healthy subject). In some embodiments, the MDS is associated with a defect in early-stage hematopoiesis (e.g., early-stage erythroid cell development, such as commitment or early differentiation, such a subject may have fewer erythroid progenitor cells in the bone marrow compared to a healthy subject or to a subject with a defect in terminal maturation). In some embodiments, the MDS is associated with elevated endogenous erythropoietin levels. In some embodiments, the MDS is associated with hypocellular bone marrow (e.g., a subject with MDS has hypocellular bone marrow). The subject may have a low transfusion burden or a high transfusion burden. In some embodiments, the subject has a low transfusion burden and received 1-3 RBC units in the eight weeks prior to treatment with an ActRII variant described herein, such as an ActRIIA variant described herein. In some embodiments, the subject has a low transfusion burden and did not receive a transfusion (received 0 RBC units) in the eight weeks prior to treatment with an ActRII variant described herein, such as an ActRIIA variant described herein. In some embodiments, the subject does not respond well to erythropoietin (EPO) or is susceptible to adverse effects of EPO (e.g., hypertension, headaches, vascular thrombosis, influenza-like syndrome, obstruction of shunts, and myocardial infarction). The compositions and methods described herein can also be used to treat subjects that do not respond to an erythroid maturation agent. In some embodiments, the subject has previously been treated with an ESA. In some embodiments, the subject has not previously been treated with an ESA. In some embodiments, the subject has not previously been treated with azacitidine, decitabine, lenalidomide, luspatercept, or sotatercept. In some embodiments, the subject has an erythropoietin level greater than 100 mIU/mL. In some embodiments, the subject has an Eastern Cooperative Oncology Group (ECOG) performance score of less than or equal to two. In some embodiments, the subject has <5% blasts in bone marrow prior to treatment with an ActRII variant described herein. In some embodiments, the subject has a peripheral blood white blood cell count less than 13,000/μL prior to treatment with an ActRII variant described herein. In some embodiments, the subject with MDS has anemia. In some embodiments, the subject with MDS has thrombocytopenia. In some embodiments, the subject with MDS has both anemia and thrombocytopenia. In some embodiments, the subject with MDS has neutropenia. In some embodiments, the subject with MDS has anemia and neutropenia. In some embodiments, the subject with MDS has thrombocytopenia and neutropenia. In some embodiments, the subject with MDS has anemia, thrombocytopenia, and neutropenia. In some embodiments, the subject with MDS has iron overload (10). In some embodiments, the subject with MDS has elevated NT-proBNP (e.g., NT-proBNP≥450 pg/mL).

A polypeptide described herein (e.g., a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)), e.g., an effective amount of an ActRIIA variant, ActRIIB variant, or ActRII chimera) can also be administered to a subject who is making a lifestyle change to treat, prevent, or reduce the risk of developing a cardiovascular disease. For example, a polypeptide containing an ActRII variant described herein can be administered to a subject who is attempting to quit smoking, become more physically active, lose weight, and/or eat a healthy diet. The combination of treatment with a polypeptide containing an ActRII variant and the lifestyle changes made by the subject may have a partially additive, wholly additive or synergistic effect.

In some embodiments, the methods described herein (e.g., the methods of treating any of the diseases or conditions described herein) do not cause any vascular complications in the subject, such as increased vascular permeability or leakage.

In any of the methods described herein, a polypeptide including an extracellular ActRII variant (e.g., an extracellular ActRIIA variant having the sequence of any one of SEQ ID NOs: 1-71 (e.g., SEQ ID NOs: 6-71) or an extracellular ActRII chimera having the sequence of any one of SEQ ID NOs: 174-216 (e.g., 195-216)) that further includes a C-terminal extension of one or more amino acids (e.g., 1, 2, 3, 4, 5, 6 or more amino acids) may be used as the therapeutic protein. In any of the methods described herein, a dimer (e.g., homodimer or heterodimer) formed by the interaction of two Fc domain monomers that are each fused to a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)) may be used as the therapeutic protein. In any of the methods described herein, a polypeptide including an extracellular ActRII variant (e.g., an extracellular ActRIIA variant having the sequence of any one of SEQ ID NOs: 1-72 (e.g., SEQ ID NOs: 6-72), an extracellular ActRIIB variant having the sequence of any one of SEQ ID NOs: 157-171 (e.g., SEQ ID NOs: 158-171), or an extracellular ActRII chimera having the sequence of any one of SEQ ID NOs: 174-216 (e.g., 195-216)) fused to an Fc domain monomer may be used as the therapeutic protein. Nucleic acids encoding the polypeptides described herein, or vectors containing said nucleic acids can also be administered according to any of the methods described herein. In any of the methods described herein, the polypeptide, nucleic acid, or vector can be administered as part of a pharmaceutical composition. Compositions that can be administered to a subject according to the methods described herein are provided in Table 10, Table 11, and Table 12, below.

TABLE 10

| Row | Composition |
|---|---|
| 1 | A polypeptide comprising an extracellular activin receptor type |
| | IIa (ActRIIa) variant, the varianthaving a sequence of |
| | GAILGRSETQECLX₁X₂NANWX₃X₄X₅X₆TNQTGVEX₇CX₈GX₉X₁₀X₁₁X₁₂X₁₃X₁₄ |
| | HCX₁₅ATWX₁₆NISGSIEIVX₁₇X₁₈GCX₁₉X₂₀X₂₁DX₂₂NCYDRTDCVEX₂₃X₂₄ |
| | X₂₅X₂₆PX₂₇VYFCCCEGNMCNEKFSYFPEMEVTQPTS (SEQ ID NO: 1), |
| | wherein X₁ is F or Y; X₂ is F or Y; X₃ is E or A; X₄ is K or L; |
| | X₅ is D or E; X₆ is R or A; X₇ is P or R; X₈ is Y or E; X₉ is D |
| | or E; X₁₀ is K or Q; X₁₁ is D or A; X₁₂ is K or A; X₁₃ is R or |
| | A; X₁₄ is R or L; X₁₅ is F or Y; X₁₆ is K, R, or A; X₁₇ is K, |
| | A, Y, F, or I; X₁₈ is Q or K; X₁₉ is W or A; X₂₀ is L or A; X₂₁ |
| | is D, K, R, A, F, G, M, N, or I; X₂₂ is I, F, or A; X₂₃ is K or |
| | T; X₂₄ is K or E; X₂₅ is D or E; X₂₆ is S or N; and X₂₇ is E or |
| | Q, and |
| | wherein the variant has at least one amino acid substitution |
| | relative to a wild-type extracellular ActRIIa having the |
| | sequence of SEQ ID NO: 73 or an extracellular ActRIIa having |
| | any one of the sequences of SEQ ID NOs: 76-96. |
| 2 | The polypeptide of row 1, wherein the variant has a sequence of |
| | GAILGRSETQECLFX₂NANWX₃X₄X₅X₆TNQTGVEX₇CX₈GX₉KX₁₁X₁₂X₁₃X₁₄HCX₁₅ |
| | ATWX₁₆NISGSIEIVX₁₇ |
| | X₁₈GCX₁₉X₂₀X₂₁DX₂₂NCYDRTDCVEX₂₃X₂₄X₂₅X₂₆PX₂₇VYFCCCEGNMCNEKFSYFP |
| | EMEVTQPTS (SEQ ID NO: 2). |
| 3 | The polypeptide of row 1 or 2, wherein the variant has a |
| | sequence of |
| | GAILGRSETQECLFX₂NANWEX₄X₅RTNQTGVEX₇CX₈GX₉KDKRX₁₄HCX₁₅ATWX₁₆NISG |
| | SIEIVKX₁₈GCWLDDX₂₂NCYDRTDCVEX₂₃X₂₄X₂₅X₂₆PX₂₇VYFCCCEGNMCNEKFSYFP |
| | EMEVTQPTS (SEQ ID NO: 3). |
| 4 | The polypeptide of any one of rows 1-3, wherein the variant |
| | has a sequence of |
| | GAILGRSETQECLFX₂NANWEX₄DRTNQTGVEX₇CX₈GX₉KDKRX₁₄HCX₁₅ATWX₁₆NISG |
| | SIEIVKX₁₈GCWLDDX₂₂NCYDRTDCVEX₂₃KX₂₅X₂₆PX₂₇VYFCCCEGNMCNEKFSYFPE |
| | MEVTQPTS (SEQ ID NO: 4). |
| 5 | The polypeptide of any one of rows 1-4, wherein the variant |
| | has a sequence of |
| | GAILGRSETQECLFX₂NANWEX₄DRTNQTGVEPCX₈GX₉KDKRX₁₄HCFATWKNISGSIEIV |
| | KX₁₈GCWLDDINCYDRTDCVEX₂₃KX₂₅X₂₆PX₂₇VYFCCCEGNMCNEKFSYFPEMEVTQPT |
| | S (SEQ ID NO: 5). |
| 6 | The polypeptide of row 1, wherein X₁ is F. |
| 7 | The polypeptide of row 1, wherein X₁ is Y. |
| 8 | The polypeptide of row 1, 6, or 7 wherein X₁₀ is K. |
| 9 | The polypeptide of row 1, 6, or 7 wherein X₁₀ is Q. |
| 10 | The polypeptide of any one of rows 1-9, wherein X₂ is F. |
| 11 | The polypeptide of any one of rows 1-9, wherein X₂ is or Y. |
| 12 | The polypeptide of any one of rows 1, 2, and 6-11, wherein |
| | X₃ is E. |
| 13 | The polypeptide of any one of rows 1, 2, and 6-11, wherein |
| | X₃ is A. |
| 14 | The polypeptide of any one of rows 1-13, wherein X₄ is K. |
| 15 | The polypeptide of any one of rows 1-13, wherein X₄ is L. |
| 16 | The polypeptide of any one of rows 1, 2, 3, and 6-15, |
| | wherein X₅ is D. |
| 17 | The polypeptide of any one of rows 1, 2, 3, and 6-15, |
| | wherein X₅ is E. |
| 18 | The polypeptide of any one of rows 1, 2, and 6-17, wherein |
| | X₆ is R. |
| 19 | The polypeptide of any one of rows 1, 2, and 6-17, wherein |
| | X₆ is A. |
| 20 | The polypeptide of any one of rows 1-4 and 6-19, wherein |
| | X₇ is P. |
| 21 | The polypeptide of any one of rows 1-4 and 6-19, wherein |
| | X₇ is R. |
| 22 | The polypeptide of any one of rows 1-21, wherein X₈ is Y. |
| 23 | The polypeptide of any one of rows 1-21, wherein X₈ is E. |
| 24 | The polypeptide of any one of rows 1-23, wherein X₉ is D. |
| 25 | The polypeptide of any one of rows 1-23, wherein X₉ is E. |
| 26 | The polypeptide of any one of rows 1, 2, and 6-25, wherein |
| | X₁₁ is D. |
| 27 | The polypeptide of any one of rows 1, 2, and 6-25, wherein |
| | X₁₁ is A. |
| 28 | The polypeptide of any one of rows 1, 2, and 6-27, wherein |
| | X₁₂ is K. |
| 29 | The polypeptide of any one of rows 1, 2, and 6-27, wherein |
| | X₁₂ is A. |
| 30 | The polypeptide of any one of rows 1, 2, and 6-29, wherein |
| | X₁₃ is R. |
| 31 | The polypeptide of any one of rows 1, 2, and 6-29, wherein |
| | X₁₃ is A. |
| 32 | The polypeptide of any one of rows 1-31, wherein X₁₄ is R. |
| 33 | The polypeptide of any one of rows 1-31, wherein X₁₄ is L. |
| 34 | The polypeptide of any one of rows 1-4 and 6-33, |
| | wherein X₁₅ is F. |
| 35 | The polypeptide of any one of rows 1-4 and 6-33, |
| | wherein X₁₅ is Y. |
| 36 | The polypeptide of any one of rows 1-4 and 6-35, |
| | wherein X₁₆ is K. |
| 37 | The polypeptide of any one of rows 1-4 and 6-35, |
| | wherein X₁₆ is R. |
| 38 | The polypeptide of any one of rows 1-4 and 6-35, |
| | wherein X₁₆ is A. |
| 39 | The polypeptide of any one of rows 1, 2, and 6-38, wherein |
| | X₁₇ is K. |
| 40 | The polypeptide of any one of rows 1, 2, and 6-38, wherein |
| | X₁₇ is A. |
| 41 | The polypeptide of any one of rows 1, 2, and 6-38, wherein |
| | X₁₇ is Y. |
| 42 | The polypeptide of any one of rows 1, 2, and 6-38, wherein |
| | X₁₇ is F. |
| 43 | The polypeptide of any one of rows 1, 2, and 6-38, wherein |
| | X₁₇ is I. |
| 44 | The polypeptide of any one of rows 1-43, wherein X₁₈ is Q. |
| 45 | The polypeptide of any one of rows 1-43, wherein X₁₈ is K. |
| 46 | The polypeptide of any one of rows 1, 2, and 6-45, wherein X₁₉ is W. |
| 47 | The polypeptide of any one of rows 1, 2, and 6-45, wherein X₁₉ is A. |
| 48 | The polypeptide of any one of rows 1, 2, and 6-47, wherein X₂₀ is L. |
| 49 | The polypeptide of any one of rows 1, 2, and 6-47, wherein X₂₀ is A. |
| 50 | The polypeptide of any one of rows 1, 2, and 6-49, wherein X₂₁ is D. |
| 51 | The polypeptide of any one of rows 1, 2, and 6-49, wherein X₂₁ is K. |
| 52 | The polypeptide of any one of rows 1, 2, and 6-49, wherein X₂₁ is R. |
| 53 | The polypeptide of any one of rows 1, 2, and 6-49, wherein X₂₁ is A. |
| 54 | The polypeptide of any one of rows 1, 2, and 6-49, wherein X₂₁ is F. |
| 55 | The polypeptide of any one of rows 1, 2, and 6-49, wherein X₂₁ is G. |
| 56 | The polypeptide of any one of rows 1, 2, and 6-49, wherein X₂₁ is M. |
| 57 | The polypeptide of any one of rows 1, 2, and 6-49, wherein X₂₁ is N. |
| 58 | The polypeptide of any one of rows 1, 2, and 6-49, wherein X₂₁ is I. |
| 59 | The polypeptide of any one of rows 1-4 and 6-58, wherein X₂₂ is I. |
| 60 | The polypeptide of any one of rows 1-4 and 6-58, wherein X₂₂ is F. |
| 61 | The polypeptide of any one of rows 1-4 and 6-58, wherein X₂₂ is A. |
| 62 | The polypeptide of any one of rows 1-61, wherein X₂₃ is K. |
| 63 | The polypeptide of any one of rows 1-61, wherein X₂₃ is T. |
| 64 | The polypeptide of any one of rows 1, 2, 3, and 6-63, wherein X₂₄ is K. |
| 65 | The polypeptide of any one of rows 1, 2, 3, and 6-63, wherein X₂₄ is E. |
| 66 | The polypeptide of any one of rows 1-65, wherein X₂₅ is D. |
| 67 | The polypeptide of any one of rows 1-65, wherein X₂₅ is E. |
| 68 | The polypeptide of any one of rows 1-67, wherein X₂₆ is S. |
| 69 | The polypeptide of any one of rows 1-67, wherein X₂₆ is N. |
| 70 | The polypeptide of any one of rows 1-69, wherein X₂₇ is E. |
| 71 | The polypeptide of any one of rows 1-69, wherein X₂₇ is Q. |
| 72 | The polypeptide of any one of rows 1-71, wherein X₂₃ is T, X₂₄ is E, |
| | X₂₅ is E, and X₂₆ is N. |
| 73 | The polypeptide of any one of rows 1-71, wherein X₂₃ is T, X₂₄ is K, |
| | X₂₅ is E, and X₂₆ is N. |
| 74 | The polypeptide of any one of rows 1-73, wherein X₁₇ is K. |
| 75 | The polypeptide of row 1, wherein the variant has the sequence of |
| | any one of SEQ ID NOs: 6-72. |
| 76 | The polypeptide of row 75, wherein the variant has the sequence of |
| | SEQ ID NO: 69. |
| 77 | The polypeptide of row 75, wherein the variant has the sequence of |
| | SEQ ID NO: 58. |
| 78 | The polypeptide of row 75, wherein the variant has the sequence of |
| | SEQ ID NO: 6. |
| 79 | The polypeptide of row 75, wherein the variant has the sequence of |
| | SEQ ID NO: 38. |
| 80 | The polypeptide of row 75, wherein the variant has the sequence of |
| | SEQ ID NO: 41. |
| 81 | The polypeptide of row 75, wherein the variant has the sequence of |
| | SEQ ID NO: 44. |
| 82 | The polypeptide of row 75, wherein the variant has the sequence of |
| | SEQ ID NO: 70. |
| 83 | The polypeptide of row 75, wherein the variant has the sequence of |
| | SEQ ID NO: 71. |
| 84 | The polypeptide of row 75, wherein the variant has the sequence of |
| | SEQ ID NO: 72. |
| 85 | The polypeptide of any one of rows 1-84, wherein the amino acid at |
| | position X₂₄ is replaced with the amino acid K. |
| 86 | The polypeptide of any one of rows 1-85, wherein the amino acid at |
| | position X₂₄ is replaced with the amino acid E. |
| 87 | The polypeptide of any one of rows 1-86, further comprising a |
| | C-terminal extension of one or more amino acids. |
| 88 | The polypeptide of row 87, wherein the C-terminal extension is NP. |
| 89 | The polypeptide of row 87, wherein the C-terminal extension is |
| | NPVTPK (SEQ ID NO: 154). |
| 90 | The polypeptide of any one of rows 1-89, further comprising an Fc |
| | domain monomer fused to the C-terminus of the polypeptide by way |
| | of a linker. |
| 91 | The polypeptide of row 90, wherein the Fc domain monomer is an IgG1 |
| | Fc domain monomer. |
| 92 | The polypeptide of row 91, wherein the IgG1 Fc domain monomer is a |
| | human IgG1 Fc domain monomer. |
| 93 | The polypeptide of row 90, wherein the Fc domain monomer comprises |
| | the sequence of SEQ ID NO: 97, SEQ ID NO: 150, or SEQ ID NO: 155. |
| 94 | The polypeptide of row 93, wherein the Fc domain monomer comprises |
| | the sequence of SEQ ID NO: 150 or SEQ ID NO: 155. |
| 95 | The polypeptide of row 94, wherein the Fc domain monomer comprises |
| | the sequence of SEQ ID NO: 155. |
| 96 | The polypeptide of row 94, wherein the Fc domain monomer comprises |
| | the sequence of SEQ ID NO: 150. |
| 97 | The polypeptide of any one of rows 90-96, wherein the polypeptide |
| | forms a dimer. |
| 98 | The polypeptide of row 97, wherein the polypeptide forms a homodimer. |
| 99 | The polypeptide of any one of rows 90-98, wherein the linker is an |
| | amino acid spacer. |
| 100 | The polypeptide of row 99, wherein the amino acid spacer is GGG, |
| | GGGA (SEQ ID NO: 98), GGGG (SEQ ID NO: 100), GGGAG (SEQ ID NO: 130), |
| | GGGAGG (SEQ ID NO: 131), or GGGAGGG (SEQ ID NO: 132). |
| 101 | The polypeptide of row 100, wherein the amino acid spacer is GGG. |
| 102 | The polypeptide of row 95 or 101, wherein the polypeptide has the |
| | sequence of SEQ ID NO: 156. |
| 103 | The polypeptide of row 99, wherein the amino acid spacer is GGGS |
| | (SEQ ID NO: 99), GGGGA (SEQ ID NO: 101), GGGGS (SEQ ID NO: 102), |
| | GGGGG (SEQ ID NO: 103), GGAG (SEQ ID NO: 104), GGSG (SEQ ID NO: |
| | 105), AGGG (SEQ ID NO: 106), SGGG (SEQ ID NO: 107), GAGA (SEQ ID |
| | NO: 108), GSGS (SEQ ID NO: 109), GAGAGA (SEQ ID NO: 110), GSGSGS |
| | (SEQ ID NO: 111), GAGAGAGA (SEQ ID NO: 112), GSGSGSGS (SEQ ID NO: |
| | 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), |
| | GGAGGA (SEQ ID NO: 118), GGSGGS (SEQ ID NO: 119), GGAGGAGGA (SEQ |
| | ID NO: 120), GGSGGSGGS (SEQ ID NO: 121), GGAGGAGGAGGA (SEQ ID NO: |
| | 122), GGSGGSGGSGGS (SEQ ID NO: 123), GGAGGGAG (SEQ ID NO: 124), |
| | GGSGGGSG (SEQ ID NO: 125), GGAGGGAGGGAG (SEQ ID NO: 126), and |
| | GGSGGGSGGGSG (SEQ ID NO: 127), GGGGAGGGGAGGGGA (SEQ ID NO: 128), |
| | GGGGSGGGGSGGGGS (SEQ ID NO: 129), AAAL (SEQ ID NO: 133), AAAK |
| | (SEQ ID NO: 134), AAAR (SEQ ID NO: 135), EGKSSGSGSESKST (SEQ ID |
| | NO: 136), GSAGSAAGSGEF (SEQ ID NO: 137), AEAAAKEAAAKA (SEQ ID |
| | NO: 138), KESGSVSSEQLAQFRSLD (SEQ ID NO: 139), GENLYFQSGG (SEQ |
| | ID NO: 140), SACYCELS (SEQ ID NO: 141), RSIAT (SEQ ID NO: 142), |
| | RPACKIPNDLKQKVMNH (SEQ ID NO: 143), |
| | GGSAGGSGSGSSGGSSGASGTGTAGGTGSGSGTGSG (SEQ ID NO: 144), |
| | AAANSSIDLISVPVDSR (SEQ ID NO: 145), |
| | GGSGGGSEGGGSEGGGSEGGGSEGGGSEGGGSGGGS (SEQ ID NO: 146), EAAAK |
| | (SEQ ID NO: 147), or PAPAP(SEQ ID NO: 148). |
| 104 | The polypeptide of any one of rows 1-103, wherein the polypeptide |
| | has a serum half-life of at least 7 days. |
| 105 | The polypeptide of any one of rows 1-104, wherein the polypeptide |
| | binds to activin A, activin B, and/or myostatin and has reduced |
| | or weak binding to human BMP9. |
| 106 | The polypeptide of row 105, wherein the polypeptide does not |
| | substantially bind to human BMP9. |
| 107 | The polypeptide of any one of rows 1-106, wherein the polypeptide |
| | binds to human activin A with a K_{D} of 800 pM or less. |
| 108 | The polypeptide of any one of rows 1-107, wherein the polypeptide |
| | binds to human activin B with a K_{D} of 800 pM or less. |
| 109 | The polypeptide of any one of rows 1-108, wherein the polypeptide |
| | binds to human GDF-11 with a K_{D} of 5 pM or higher. |
| 110 | A nucleic acid molecule encoding a polypeptide of any one of rows |
| | 1-109. |
| 111 | A vector comprising the nucleic acid molecule of row 110. |
| 112 | A host cell that expresses a polypeptide of any one of rows 1-109, |
| | wherein the host cell comprises a nucleic acid molecule of row 110 |
| | or a vector of row 111, wherein the nucleic acid molecule or vector |
| | is expressed in the host cell. |
| 113 | A pharmaceutical composition comprising a polypeptide of any one of |
| | rows 1-109, a nucleic acid molecule of row 110, or a vector of row |
| | 111, and one or more pharmaceutically acceptable carriers or |
| | excipients. |
| 114 | The pharmaceutical composition of row 113, wherein the polypeptide |
| | is in a therapeutically effective amount. |
| 115 | A construct comprising two identical polypeptides (e.g., a homodimer), |
| | each comprising an extracellular ActRIIA variant of any one of rows |
| | 1-89 (e.g., an ActRIIA variant having a sequence of any one of SEQ |
| | ID NOs: 1-72) fused (e.g., linked using an amino acid spacer) to the |
| | N- or C-terminus of an Fc domain monomer (e.g., the sequence of SEQ |
| | ID NO: 97, SEQ ID NO: 150, or SEQ ID NO: 155). The two Fc domain |
| | monomers in the two polypeptides interact to form an Fc domain in |
| | the construct. |
| 116 | A construct comprising two different polypeptides (e.g., a heterodimer), |
| | each comprising an extracellular ActRIIA variant of any one of rows |
| | 1-89 (e.g., an ActRIIA variant having a sequence of any one of SEQ |
| | ID NOs: 1-72) fused (e.g., linked using an amino acid spacer) to the |
| | N- or C-terminus of an Fc domain monomer (e.g., the sequence of SEQ |
| | ID NO: 97, SEQ ID NO: 150, or SEQ ID NO: 155). The two Fc domain |
| | monomers in the two polypeptides interact to form an Fc domain in |
| | the construct. |

TABLE 11

| Row | Composition |
|---|---|
| 1 | A polypeptide comprising an extracellular activin receptor type IIB (ActRIIB) variant, the |
| | variant having one or more amino acid substitutions relative to the sequence of |
| | GRGEAETRECIYYNANWELERTNQSGLERCEGEQDKRLHCYASWRNSSGTIELVKKGCWL |
| | DDFNCYDRQECVATEENPQVYFCCCEGNFCNERFTHLPEAGGPEVTYEPPPTAPT (SEQ ID |
| | NO: 74), wherein the variant comprises one or more amino acid substitutions that impart |
| | reduced BMP9 binding relative to wild type extracellular ActRIIB and one or more additional |
| | amino acid substitutions, wherein the substitutions that reduce BMP9 binding comprise one or |
| | more of: |
| | a) amino acid substitution E75K; |
| | b) amino acid substitutions Q69T and E70D; or |
| | c) amino acid substitutions Q69D and E70T, |
| | optionally wherein the variant is truncated from the N-terminus by deletion of 1, 2, 3, 4, 5, 6, |
| | or 7 amino acids. |
| 2 | The polypeptide of row 1, wherein the variant comprises one or more amino acid substitutions |
| | selected from the group consisting of I11L, Y12F, L19K, E20D, S25T, L27V, R29P, E31Y, |
| | E33D, Q34K, L38R, Y41F, R45K, S47I, S48T, T50S, I51L, L53I, K56Q, F63I, T74K, E76D, |
| | N77S, Q79E, and F89M. |
| 3 | The polypeptide of row 1 or 2, wherein the variant comprises amino acid substitutions E75K, |
| | E20D, and F63I. |
| 4 | The polypeptide of row 1 or 2, wherein the variant comprises amino acid substitution E75K. |
| 5 | The polypeptide of row 4, wherein the variant comprises amino acid substitutions T74K, E76D, |
| | N77S, and Q79E. |
| 6 | The polypeptide of row 5, wherein the variant further comprises one or more additional amino |
| | acid substitutions. |
| 7 | The polypeptide of row 6, wherein the variant comprises amino acid substitutions Y41F, R45K, |
| | and K56Q. |
| 8 | The polypeptide of row 7, wherein the variant further comprises amino acid substitutions |
| | Y12F, L19K, E20D, R29P, E31Y, E33D, L38R, and F63I. |
| 9 | The polypeptide of row 6, wherein the variant comprises amino acid substitutions S25T and |
| | S47I. |
| 10 | The polypeptide of row 9, wherein the variant comprises amino acid substitution S48T. |
| 11 | The polypeptide of row 6, wherein the variant comprises amino acid substitution R29P. |
| 12 | The polypeptide of row 6, wherein the variant comprises amino acid substitutions E31Y, |
| | E33D, and Q34K. |
| 13 | The polypeptide of row 6, wherein the variant comprises amino acid substitutions Y12F, L19K, |
| | and E20D. |
| 14 | The polypeptide of row 6, wherein the variant comprises amino acid substitutions E31Y, |
| | E33D, and L38R. |
| 15 | The polypeptide of row 1 or 2, wherein the variant comprises amino acid substitutions Q69T |
| | and E70D. |
| 16 | The polypeptide of any one of rows 1, 2, and 15, wherein the variant comprises amino acid |
| | substitutions Q69T and E70D and additional amino acid substitutions I11L, L27V, Q34K, |
| | T50S, I51L, L53I, and F89M. |
| 17 | The polypeptide of row 1 or 2, wherein the variant comprises amino acid substitutions Q69D |
| | and E70T. |
| 18 | The polypeptide of any one of rows 1, 2, and 17, wherein the variant comprises amino acid |
| | substitutions Q69D and E70T and additional amino acid substitutions I11L, L27V, Q34K, |
| | T50S, I51L, L53I, and F89M. |
| 19 | The polypeptide of any one of rows 15-18, wherein the variant comprises amino acid |
| | substitution E75K. |
| 20 | A polypeptide comprising an ActRIIB variant, the variant having a sequence of |
| | GRGEAETRECX₁X₂YNANWEX₃X₄RTNQX₅GX₆EX₇CX₈GX₉X₁₀DKRX₁₁HCX₁₂ASWX₁₃NX₁₄X₁₅ |
| | GX₁₆X₁₇EX₁₈VKX₁₉GCWLDDX₂₀NCYDRX₂₁X₂₂CVAX₂₃X₂₄X₂₅X₂₆PX₂₇VYFCCCEGNX₂₈CNERF |
| | THLPEAGGPEVTYEPPPTAPT (SEQ ID NO: 157), wherein X₁ is I or L; X₂ is F or Y; X₃ is L or |
| | K; X₄ is D or E; X₅ is T or S; X₆ is L or V; X₇ is P or R; X₈ is Y or E; X₉ is D or E; X₁₀ is K or |
| | Q; X₁₁ is R or L; X₁₂ is Y or F; X₁₃ is R or K; X₁₄ is S or I; X₁₅ is S or T; X₁₆ is S or T; X₁₇ is |
| | I or L; X₁₈ is I or L; X₁₉ is K or Q; X₂₀ is F or I; X₂₁ is Q, T, or D; X₂₂ is E, D, or T; X₂₃ is K |
| | or T; X₂₄ is K or E; X₂₅ is D or E; X₂₆ is S or N; X₂₇ is E or Q; and X₂₈ is F or M, and wherein |
| | X₂₄ is E and/or either X₂₁ is T and X₂₂ is D or X₂₁ is D and X₂₂ is T, and wherein the variant has |
| | at least one amino acid substitution relative to a wild-type extracellular ActRIIB having the |
| | sequence of SEQ ID NO: 74, optionally wherein the variant is truncated from the N-terminus by |
| | deletion of 1, 2, 3, 4, 5, 6, or 7 amino acids. |
| 21 | The polypeptide of row 20, wherein X₁ is I. |
| 22 | The polypeptide of row 20, wherein X₁ is L. |
| 23 | The polypeptide of any one of rows 20-22, wherein X₂ is F. |
| 24 | The polypeptide of any one of rows 20-22, wherein X₂ is Y. |
| 25 | The polypeptide of any one of rows 20-24, wherein X₃ is L. |
| 26 | The polypeptide of any one of rows 20-24, wherein X₃ is K. |
| 27 | The polypeptide of any one of rows 20-26, wherein X₄ is D. |
| 28 | The polypeptide of any one of rows 20-26, wherein X₄ is E. |
| 29 | The polypeptide of any one of rows 20-28, wherein X₅ is T. |
| 30 | The polypeptide of any one of rows 20-28, wherein X₅ is S. |
| 31 | The polypeptide of any one of rows 20-30, wherein X₆ is L. |
| 32 | The polypeptide of any one of rows 20-30, wherein X₆ is V. |
| 33 | The polypeptide of any one of rows 20-32, wherein X₇ is P. |
| 34 | The polypeptide of any one of rows 20-32, wherein X₇ is R. |
| 35 | The polypeptide of any one of rows 20-34, wherein X₈ is Y. |
| 36 | The polypeptide of any one of rows 20-34, wherein X₈ is E. |
| 37 | The polypeptide of any one of rows 20-36, wherein X₉ is D. |
| 38 | The polypeptide of any one of rows 20-36, wherein X₉ is E. |
| 39 | The polypeptide of any one of rows 20-38, wherein X₁₀ is K. |
| 40 | The polypeptide of any one of rows 20-38, wherein X₁₀ is Q. |
| 41 | The polypeptide of any one of rows 20-40, wherein X₁₁ is R. |
| 42 | The polypeptide of any one of rows 20-40, wherein X₁₁ is L. |
| 43 | The polypeptide of any one of rows 20-42, wherein X₁₂ is Y. |
| 44 | The polypeptide of any one of rows 20-42, wherein X₁₂ is F. |
| 45 | The polypeptide of any one of rows 20-44, wherein X₁₃ is R. |
| 46 | The polypeptide of any one of rows 20-44, wherein X₁₃ is K. |
| 47 | The polypeptide of any one of rows 20-46, wherein X₁₄ is S. |
| 48 | The polypeptide of any one of rows 20-46, wherein X₁₄ is I. |
| 49 | The polypeptide of any one of rows 20-48, wherein X₁₅ is S. |
| 50 | The polypeptide of any one of rows 20-48, wherein X₁₅ is T. |
| 51 | The polypeptide of any one of rows 20-50, wherein X₁₆ is S. |
| 52 | The polypeptide of any one of rows 20-50, wherein X₁₆ is T. |
| 53 | The polypeptide of any one of rows 20-52, wherein X₁₇ is I. |
| 54 | The polypeptide of any one of rows 20-52, wherein X₁₇ is L. |
| 55 | The polypeptide of any one of rows 20-54, wherein X₁₈ is I. |
| 56 | The polypeptide of any one of rows 20-54, wherein X₁₈ is L. |
| 57 | The polypeptide of any one of rows 20-56, wherein X₁₉ is K. |
| 58 | The polypeptide of any one of rows 20-56, wherein X₁₉ is Q. |
| 59 | The polypeptide of any one of rows 20-58, wherein X₂₀ is F. |
| 60 | The polypeptide of any one of rows 20-58, wherein X₂₀ is I. |
| 61 | The polypeptide of any one of rows 20-60, wherein X₂₁ is Q. |
| 62 | The polypeptide of any one of rows 20-60, wherein X₂₁ is T. |
| 63 | The polypeptide of any one of rows 20-60, wherein X₂₁ is D. |
| 64 | The polypeptide of any one of rows 20-61, wherein X₂₂ is E. |
| 65 | The polypeptide of any one of rows 20-60 and 62, wherein X₂₂ is D. |
| 66 | The polypeptide of any one of rows 20-60 and 63, wherein X₂₂ is T. |
| 67 | The polypeptide of any one of rows 20-66, wherein X₂₃ is K. |
| 68 | The polypeptide of any one of rows 20-66, wherein X₂₃ is T. |
| 69 | The polypeptide of any one of rows 20-68, wherein X₂₄ is K. |
| 70 | The polypeptide of any one of rows 20-60, 62, 63, and 65-68, wherein X₂₄ is E. |
| 71 | The polypeptide of any one of rows 20-70, wherein X₂₅ is D. |
| 72 | The polypeptide of any one of rows 20-70, wherein X₂₅ is E. |
| 73 | The polypeptide of any one of rows 20-72, wherein X₂₆ is S. |
| 74 | The polypeptide of any one of rows 20-72, wherein X₂₆ is N. |
| 75 | The polypeptide of any one of rows 20-74, wherein X₂₇ is E. |
| 76 | The polypeptide of any one of rows 20-74, wherein X₂₇ is Q. |
| 77 | The polypeptide of any one of rows 20-76, wherein X₂₈ is F. |
| 78 | The polypeptide of any one of rows 20-76, wherein X₂₈ is M. |
| 79 | The polypeptide of any one of rows 20-78, wherein X₂₃ is T, X₂₄ is K, X₂₅ is E, and X₂₆ is N. |
| 80 | The polypeptide of any one of rows 20-78, wherein X₂₃ is T, X₂₄ is E, X₂₅ is E, and X₂₆ is N. |
| 81 | The polypeptide of any one of rows 20-78, wherein X₂₃ is K, X₂₄ is K, X₂₅ is D, and X₂₆ is S. |
| 82 | The polypeptide of any one of rows 1-81, wherein the variant has the sequence of any one of |
| | SEQ ID NOs: 158-171. |
| 83 | The polypeptide of row 82, wherein the variant has the sequence of SEQ ID NO: 159. |
| 84 | The polypeptide of row 82, wherein the variant has the sequence of SEQ ID NO: 165. |
| 85 | The polypeptide of row 82, wherein the variant has the sequence of SEQ ID NO: 168. |
| 86 | The polypeptide of row 82, wherein the variant has the sequence of SEQ ID NO: 169. |
| 87 | The polypeptide of row 82, wherein the variant has the sequence of SEQ ID NO: 170. |
| 88 | The polypeptide of row 82, wherein the variant has the sequence of SEQ ID NO: 171. |
| 89 | The polypeptide of any one of rows 1-88, wherein the amino acid at position X₂₄ is replaced |
| | with the amino acid K. |
| 90 | The polypeptide of any one of rows 1-88, wherein the amino acid at position X₂₄ is replaced |
| | with the amino acid E. |
| 91 | The polypeptide of any one of rows 1-90, wherein the variant is truncated from the N-terminus |
| | by deletion of one amino acid. |
| 92 | The polypeptide of any one of rows 1-90, wherein the variant is truncated from the N-terminus |
| | by deletion of two amino acids. |
| 93 | The polypeptide of any one of rows 1-90, wherein the variant is truncated from the N-terminus |
| | by deletion of three amino acids. |
| 94 | The polypeptide of any one of rows 1-90, wherein the variant is truncated from the N-terminus |
| | by deletion of four amino acids. |
| 95 | The polypeptide of any one of rows 1-90, wherein the variant is truncated from the N-terminus |
| | by deletion of five amino acids. |
| 96 | The polypeptide of any one of rows 1-90, wherein the variant is truncated from the N-terminus |
| | by deletion of six amino acids. |
| 97 | The polypeptide of any one of rows 1-90, wherein the variant is truncated from the N-terminus |
| | by deletion of seven amino acids. |
| 98 | The polypeptide of any one of rows 1-97, further comprising an Fc domain monomer fused to |
| | the C-terminus of the polypeptide by way of a linker. |
| 99 | The polypeptide of row 98, wherein the Fc domain monomer is an IgG1 Fc domain monomer. |
| 100 | The polypeptide of row 99, wherein the IgG1 Fc domain monomer is a human IgG1 Fc domain |
| | monomer. |
| 101 | The polypeptide of row 98, wherein the Fc domain monomer comprises the sequence of SEQ |
| | ID NO: 97, SEQ ID NO: 150, or SEQ ID NO: 155. |
| 102 | The polypeptide of row 101, wherein the Fc domain monomer comprises the sequence of |
| | SEQ ID NO: 150 or SEQ ID NO: 155. |
| 103 | The polypeptide of row 102, wherein the Fc domain monomer comprises the sequence of |
| | SEQ ID NO: 150. |
| 104 | The polypeptide of row 102, wherein the Fc domain monomer comprises the sequence of |
| | SEQ ID NO: 155. |
| 105 | The polypeptide of any one of rows 98-104, wherein the polypeptide forms a dimer. |
| 106 | The polypeptide of row 105, wherein the polypeptide forms a homodimer. |
| 107 | The polypeptide of any one of rows 98-106, wherein the linker is an amino acid spacer. |
| 108 | The polypeptide of row 107, wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 109 | The polypeptide of row 108, wherein the amino acid spacer is GGG. |
| 110 | The polypeptide of row 103 or 109, wherein the polypeptide has the sequence of SEQ ID NO: |
| | 172. |
| 111 | The polypeptide of row 107, wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG |
| | (SEQ ID NO: 103), GGAG (SEQ ID NO: 104), GGSG (SEQ ID NO: 105), AGGG (SEQ ID NO: |
| | 106), SGGG (SEQ ID NO: 107), GAGA (SEQ ID NO: 108), GSGS (SEQ ID NO: 109), |
| | GAGAGA (SEQ ID NO: 110), GSGSGS (SEQ ID NO: 111), GAGAGAGA (SEQ ID NO: 112), |
| | GSGSGSGS (SEQ ID NO: 113), GAGAGAGAGA (SEQ ID NO: 114), GSGSGSGSGS (SEQ |
| | ID NO: 115), GAGAGAGAGAGA (SEQ ID NO: 116), and GSGSGSGSGSGS (SEQ ID NO: |
| | 117), GGAGGA (SEQ ID NO: 118), GGSGGS (SEQ ID NO: 119), GGAGGAGGA (SEQ ID |
| | NO: 120), GGSGGSGGS (SEQ ID NO: 121), GGAGGAGGAGGA (SEQ ID NO: 122), |
| | GGSGGSGGSGGS (SEQ ID NO: 123), GGAGGGAG (SEQ ID NO: 124), GGSGGGSG (SEQ |
| | ID NO: 125), GGAGGGAGGGAG (SEQ ID NO: 126), and GGSGGGSGGGSG (SEQ ID NO: |
| | 127), GGGGAGGGGAGGGGA (SEQ ID NO: 128), GGGGSGGGGSGGGGS (SEQ ID NO: |
| | 129), AAAL (SEQ ID NO: 133), AAAK (SEQ ID NO: 134), AAAR (SEQ ID NO: 135), |
| | EGKSSGSGSESKST (SEQ ID NO: 136), GSAGSAAGSGEF (SEQ ID NO: 137), |
| | AEAAAKEAAAKA (SEQ ID NO: 138), KESGSVSSEQLAQFRSLD (SEQ ID NO: 139), |
| | GENLYFQSGG (SEQ ID NO: 140), SACYCELS (SEQ ID NO: 141), RSIAT (SEQ ID NO: 142), |
| | RPACKIPNDLKQKVMNH (SEQ ID NO: 143), |
| | GGSAGGSGSGSSGGSSGASGTGTAGGTGSGSGTGSG (SEQ ID NO: 144), |
| | AAANSSIDLISVPVDSR (SEQ ID NO: 145), |
| | GGSGGGSEGGGSEGGGSEGGGSEGGGSEGGGSGGGS (SEQ ID NO: 146), EAAAK (SEQ |
| | ID NO: 147), or PAPAP(SEQ ID NO: 148). |
| 112 | The polypeptide of any one of rows 1-111, wherein the polypeptide has a serum half-life of at |
| | least 7 days. |
| 113 | The polypeptide of any one of rows 1-112, wherein the polypeptide binds to activin A, activin |
| | B, and/or myostatin and has reduced or weak binding to human BMP9. |
| 114 | The polypeptide of row 113, wherein the polypeptide does not substantially bind to human |
| | BMP9. |
| 115 | The polypeptide of any one of rows 1-114, wherein the polypeptide binds to human activin A |
| | with a K_{D} of 800 pM or less. |
| 116 | The polypeptide of any one of rows 1-115, wherein the polypeptide binds to human activin B |
| | with a K_{D} of 800 pM or less. |
| 117 | The polypeptide of any one of rows 1-116, wherein the polypeptide binds to human GDF-11 |
| | with a K_{D} of 5 pM or higher. |
| 118 | A nucleic acid molecule encoding a polypeptide of any one of rows 1-117. |
| 119 | A vector comprising the nucleic acid molecule of row 118. |
| 120 | A host cell that expresses a polypeptide of any one of rows 1-117, wherein the host cell |
| | comprises a nucleic acid molecule of row 118 or a vector of row 119, wherein the nucleic acid |
| | molecule or vector is expressed in the host cell. |
| 121 | A pharmaceutical composition comprising a polypeptide of any one of rows 1-117, a nucleic |
| | acid molecule of row 118, or a vector of row 119, and one or more pharmaceutically |
| | acceptable carriers or excipients. |
| 122 | The pharmaceutical composition of row 121, wherein the polypeptide is in a therapeutically |
| | effective amount. |
| 123 | A construct comprising two identical polypeptides (e.g., a homodimer), each comprising an |
| | extracellular ActRIIB variant of any one of rows 1-97 (e.g., an ActRIIB variant having a |
| | sequence of any one of SEQ ID NOs: 157-171) fused (e.g., linked using an amino acid |
| | spacer) to the N- or C-terminus of an Fc domain monomer (e.g., the sequence of SEQ ID NO: |
| | 97, SEQ ID NO: 150, or SEQ ID NO: 155). The two Fc domain monomers in the two |
| | polypeptides interact to form an Fc domain in the construct. |
| 124 | A construct comprising two different polypeptides (e.g., a heterodimer), each comprising an |
| | extracellular ActRIIB variant of any one of rows 1-97 (e.g., an ActRIIB variant having a |
| | sequence of any one of SEQ ID NOs: 157-171) fused (e.g., linked using an amino acid |
| | spacer) to the N- or C-terminus of an Fc domain monomer (e.g., the sequence of SEQ ID NO: |
| | 97, SEQ ID NO: 150, or SEQ ID NO: 155). The two Fc domain monomers in the two |
| | polypeptides interact to form an Fc domain in the construct. |

TABLE 12

| Row | Composition |
|---|---|
| 1 | A polypeptide comprising an extracellular activin receptor type II (ActRII) chimera, the |
| | chimera having a sequence of any one of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 174), |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 175), |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 176), |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 177), |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 178), |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 179), |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 180), |
| | GRGEAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 181), |
| | GRGEAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 182), |
| | GRGEAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 183), |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 184), |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 185), |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 186), |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 187), |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 188), |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 189), |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 190), |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 191), |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 192), |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 193), |
| | and |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 194), |
| | wherein X₁ is D or R, X₂ is I, F, E, D, Y, S, N, Q, or T, X₃ is N or T, X₄ is A or E, X₅ is T or |
| | K, X₆ is E or K, X₇ is E or D, X₈ is N or S, and X₉ is Q, E, K, R, D, or N, optionally wherein |
| | the chimera is truncated from the N-terminus by deletion of 1, 2, 3, 4, 5, 6, 7, 8, or 9 amino |
| | acids, wherein the chimera retains the two amino acids before the first cysteine. |
| 2 | The polypeptide of row 1, wherein the chimera has the sequence of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 174). |
| 3 | The polypeptide of row 1, wherein the chimera has the sequence of |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 175). |
| 4 | The polypeptide of row 1, wherein the chimera has the sequence of |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 176). |
| 5 | The polypeptide of row 1, wherein the chimera has the sequence of |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 177). |
| 6 | The polypeptide of row 1, wherein the chimera has the sequence of |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 178). |
| 7 | The polypeptide of row 1, wherein the chimera has the sequence of |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 179). |
| 8 | The polypeptide of row 1, wherein the chimera has the sequence of |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 180). |
| 9 | The polypeptide of row 1, wherein the chimera has the sequence of |
| | GRGEAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 181). |
| 10 | The polypeptide of row 1, wherein the chimera has the sequence of |
| | GRGEAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 182). |
| 11 | The polypeptide of row 1, wherein the chimera has the sequence of |
| | GRGEAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 183). |
| 12 | The polypeptide of row 1, wherein the chimera has the sequence of |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 184). |
| 13 | The polypeptide of row 1, wherein the chimera has the sequence of |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 185). |
| 14 | The polypeptide of row 1, wherein the chimera has the sequence of |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 186). |
| 15 | The polypeptide of row 1, wherein the chimera has the sequence of |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 187). |
| 16 | The polypeptide of row 1, wherein the chimera has the sequence of |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 188). |
| 17 | The polypeptide of row 1, wherein the chimera has the sequence of |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 189). |
| 18 | The polypeptide of row 1, wherein the chimera has the sequence of |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 190). |
| 19 | The polypeptide of row 1, wherein the chimera has the sequence of |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 191). |
| 20 | The polypeptide of row 1, wherein the chimera has the sequence of |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 192). |
| 21 | The polypeptide of row 1, wherein the chimera has the sequence of |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 193). |
| 22 | The polypeptide of row 1, wherein the chimera has the sequence of |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 194). |
| 23 | The polypeptide of any one of rows 1-22, wherein X₁ is D. |
| 24 | The polypeptide of any one of rows 1-22, wherein X₁ is R. |
| 25 | The polypeptide of any one of rows 1-24, wherein X₂ is I. |
| 26 | The polypeptide of any one of rows 1-24, wherein X₂ is F. |
| 27 | The polypeptide of any one of rows 1-24, wherein X₂ is E. |
| 28 | The polypeptide of any one of rows 1-24, wherein X₂ is D. |
| 29 | The polypeptide of any one of rows 1-24, wherein X₂ is Y. |
| 30 | The polypeptide of any one of rows 1-24, wherein X₂ is S. |
| 31 | The polypeptide of any one of rows 1-24, wherein X₂ is N. |
| 32 | The polypeptide of any one of rows 1-24, wherein X₂ is Q. |
| 33 | The polypeptide of any one of rows 1-24, wherein X₂ is T. |
| 34 | The polypeptide of any one of rows 1-33, wherein X₃ is N. |
| 35 | The polypeptide of any one of rows 1-33, wherein X₃ is T. |
| 36 | The polypeptide of any one of rows 1-35, wherein X₄ is A. |
| 37 | The polypeptide of any one of rows 1-35, wherein X₄ is E. |
| 38 | The polypeptide of any one of rows 1-37, wherein X₅ is T. |
| 39 | The polypeptide of any one of rows 1-37, wherein X₅ is K. |
| 40 | The polypeptide of any one of rows 1-39, wherein X₆ is E. |
| 41 | The polypeptide of any one of rows 1-39, wherein X₆ is K. |
| 42 | The polypeptide of any one of rows 1-41, wherein X₇ is E. |
| 43 | The polypeptide of any one of rows 1-41, wherein X₇ is D. |
| 44 | The polypeptide of any one of rows 1-43, wherein X₈ is N. |
| 45 | The polypeptide of any one of rows 1-43, wherein X₈ is S. |
| 46 | The polypeptide of any one of rows 1-45, wherein X₉ is Q. |
| 47 | The polypeptide of any one of rows 1-45, wherein X₉ is E. |
| 48 | The polypeptide of any one of rows 1-45, wherein X₉ is K. |
| 49 | The polypeptide of any one of rows 1-45, wherein X₉ is R. |
| 50 | The polypeptide of any one of rows 1-45, wherein X₉ is D. |
| 51 | The polypeptide of any one of rows 1-45, wherein X₉ is N. |
| 52 | The polypeptide of any one of rows 1-51, wherein X₅ is T, X₆ is E, X₇ is E, and X₈ is N. |
| 53 | The polypeptide of any one of rows 1-51, wherein X₅ is T, X₆ is K, X₇ is E, and X₈ is N. |
| 54 | The polypeptide of any one of rows 1-53, wherein X₂ is E and X₃ is T. |
| 55 | The polypeptide of any one of rows 1-53, wherein X₂ is I or F and X₃ is N. |
| 56 | The polypeptide of row 55, wherein X₂ is I. |
| 57 | The polypeptide of row 55, wherein X₂ is F. |
| 58 | The polypeptide of row 1, wherein the chimera has the sequence of any one of SEQ ID NOS: |
| | 195-216. |
| 59 | The polypeptide of row 58, wherein the chimera has the sequence of SEQ ID NO: 195. |
| 60 | The polypeptide of row 58, wherein the chimera has the sequence of SEQ ID NO: 196. |
| 61 | The polypeptide of row 58, wherein the chimera has the sequence of SEQ ID NO: 197. |
| 62 | The polypeptide of row 58, wherein the chimera has the sequence of SEQ ID NO: 198. |
| 63 | The polypeptide of row 58, wherein the chimera has the sequence of SEQ ID NO: 213. |
| 64 | The polypeptide of row 58, wherein the chimera has the sequence of SEQ ID NO: 214. |
| 65 | The polypeptide of row 58, wherein the chimera has the sequence of SEQ ID NO: 201. |
| 66 | The polypeptide of row 58, wherein the chimera has the sequence of SEQ ID NO: 215. |
| 67 | The polypeptide of row 58, wherein the chimera has the sequence of SEQ ID NO: 216. |
| 68 | The polypeptide of row 58, wherein the chimera has the sequence of SEQ ID NO: 210. |
| 69 | The polypeptide of any one of rows 1-68, wherein the chimera is truncated from the N-terminus |
| | by deletion of one amino acid. |
| 70 | The polypeptide of any one of rows 1-68, wherein the chimera is truncated from the N-terminus |
| | by deletion of two amino acids. |
| 71 | The polypeptide of any one of rows 1-68, wherein the chimera is truncated from the N-terminus |
| | by deletion of three amino acids. |
| 72 | The polypeptide of any one of rows 1-68, wherein the chimera is truncated from the N-terminus |
| | by deletion of four amino acids. |
| 73 | The polypeptide of any one of rows 1-68, wherein the chimera is truncated from the N-terminus |
| | by deletion of five amino acids. |
| 74 | The polypeptide of any one of rows 1-68, wherein the chimera is truncated from the N-terminus |
| | by deletion of six amino acids. |
| 75 | The polypeptide of any one of rows 1-68, wherein the chimera is truncated from the N-terminus |
| | by deletion of seven amino acids. |
| 76 | The polypeptide of any one of rows 1-68, wherein the chimera is truncated from the N-terminus |
| | by deletion of eight amino acids. |
| 77 | The polypeptide of any one of rows 1-68, wherein the chimera is truncated from the N-terminus |
| | by deletion of nine amino acids. |
| 78 | The polypeptide of any one of rows 1 and 69-77, wherein the chimera has the sequence of any |
| | one of SEQ ID NOs: 221-293. |
| 79 | The polypeptide of row 78, wherein the chimera has the sequence of SEQ ID NO: 221. |
| 80 | The polypeptide of row 78, wherein the chimera has the sequence of SEQ ID NO: 226. |
| 81 | The polypeptide of row 78, wherein the chimera has the sequence of SEQ ID NO: 227. |
| 82 | The polypeptide of row 78, wherein the chimera has the sequence of SEQ ID NO: 228. |
| 83 | The polypeptide of row 78, wherein the chimera has the sequence of SEQ ID NO: 229. |
| 84 | The polypeptide of row 78, wherein the chimera has the sequence of SEQ ID NO: 230. |
| 85 | The polypeptide of row 78, wherein the chimera has the sequence of SEQ ID NO: 231. |
| 86 | The polypeptide of row 78, wherein the chimera has the sequence of SEQ ID NO: 232. |
| 87 | The polypeptide of row 78, wherein the chimera has the sequence of SEQ ID NO: 236. |
| 88 | The polypeptide of row 78, wherein the chimera has the sequence of SEQ ID NO: 240. |
| 89 | The polypeptide of row 78, wherein the chimera has the sequence of SEQ ID NO: 252. |
| 90 | The polypeptide of row 78, wherein the chimera has the sequence of SEQ ID NO: 253. |
| 91 | The polypeptide of row 78, wherein the chimera has the sequence of SEQ ID NO: 256. |
| 92 | The polypeptide of row 78, wherein the chimera has the sequence of SEQ ID NO: 257. |
| 93 | The polypeptide of row 78, wherein the chimera has the sequence of SEQ ID NO: 259. |
| 94 | The polypeptide of row 78, wherein the chimera has the sequence of SEQ ID NO: 260. |
| 95 | The polypeptide of row 78, wherein the chimera has the sequence of SEQ ID NO: 261. |
| 96 | The polypeptide of row 78, wherein the chimera has the sequence of SEQ ID NO: 262. |
| 97 | The polypeptide of row 78, wherein the chimera has the sequence of SEQ ID NO: 263. |
| 98 | The polypeptide of row 78, wherein the chimera has the sequence of SEQ ID NO: 264. |
| 99 | The polypeptide of row 78, wherein the chimera has the sequence of SEQ ID NO: 265. |
| 100 | The polypeptide of row 78, wherein the chimera has the sequence of SEQ ID NO: 266. |
| 101 | The polypeptide of row 78, wherein the chimera has the sequence of SEQ ID NO: 268. |
| 102 | The polypeptide of row 78, wherein the chimera has the sequence of SEQ ID NO: 269. |
| 103 | The polypeptide of row 78, wherein the chimera has the sequence of SEQ ID NO: 271. |
| 104 | The polypeptide of row 78, wherein the chimera has the sequence of SEQ ID NO: 272. |
| 105 | The polypeptide of row 78, wherein the chimera has the sequence of SEQ ID NO: 273. |
| 106 | The polypeptide of row 78, wherein the chimera has the sequence of SEQ ID NO: 274. |
| 107 | The polypeptide of row 78, wherein the chimera has the sequence of SEQ ID NO: 277. |
| 108 | The polypeptide of row 78, wherein the chimera has the sequence of SEQ ID NO: 281. |
| 109 | The polypeptide of row 78, wherein the chimera has the sequence of SEQ ID NO: 282. |
| 110 | The polypeptide of row 78, wherein the chimera has the sequence of SEQ ID NO: 283. |
| 111 | The polypeptide of row 78, wherein the chimera has the sequence of SEQ ID NO: 286. |
| 112 | The polypeptide of row 78, wherein the chimera has the sequence of SEQ ID NO: 287. |
| 113 | The polypeptide of row 78, wherein the chimera has the sequence of SEQ ID NO: 289. |
| 114 | The polypeptide of row 78, wherein the chimera has the sequence of SEQ ID NO: 293. |
| 115 | The polypeptide of any one of rows 1-114, wherein the polypeptide (e.g., the chimera) further |
| | includes a C-terminal extension of one or more amino acids (e.g., 1, 2, 3, 4, 5, 6, or more |
| | amino acids from wild-type extracellular ActRIIA or ActRIIB). |
| 116 | The polypeptide of row 115, wherein the C-terminal extension is NP. |
| 117 | The polypeptide of row 115, wherein the C-terminal extension is NPVTPK (SEQ ID NO: 154). |
| 118 | The polypeptide of any one of rows 1-117, wherein the polypeptide further includes an Fc domain |
| | monomer fused to the C-terminus of the polypeptide (e.g., the C-terminus of the chimera) by way |
| | of a linker. |
| 119 | The polypeptide of row 118, wherein the Fc domain monomer is an IgG1 Fc domain monomer. |
| 120 | The polypeptide of row 119, wherein the IgG1 Fc domain monomer is a human IgG1 Fc domain |
| | monomer. |
| 121 | The polypeptide of row 118, wherein the Fc domain monomer has the sequence of SEQ ID NO: |
| | 97, SEQ ID NO: 150, or SEQ ID NO: 155. |
| 122 | The polypeptide of row 121, wherein the Fc domain monomer has the sequence of SEQ ID NO: |
| | 150 or SEQ ID NO: 155. |
| 123 | The polypeptide of any one of rows 118-122, wherein the polypeptide has the sequence of any |
| | one of SEQ ID NOs: 217-220 and SEQ ID NOs: 294-373. |
| 124 | The polypeptide of row 123, wherein the polypeptide has the sequence of SEQ ID NO: 217. |
| 125 | The polypeptide of row 123, wherein the polypeptide has the sequence of SEQ ID NO: 218. |
| 126 | The polypeptide of row 123, wherein the polypeptide has the sequence of SEQ ID NO: 219. |
| 127 | The polypeptide of row 123, wherein the polypeptide has the sequence of SEQ ID NO: 220. |
| 128 | The polypeptide of row 123, wherein the polypeptide has the sequence of SEQ ID NO: 294. |
| 129 | The polypeptide of row 123, wherein the polypeptide has the sequence of SEQ ID NO: 299. |
| 130 | The polypeptide of row 123, wherein the polypeptide has the sequence of SEQ ID NO: 300. |
| 131 | The polypeptide of row 123, wherein the polypeptide has the sequence of SEQ ID NO: 301. |
| 132 | The polypeptide of row 123, wherein the polypeptide has the sequence of SEQ ID NO: 302. |
| 133 | The polypeptide of row 123, wherein the polypeptide has the sequence of SEQ ID NO: 303. |
| 134 | The polypeptide of row 123, wherein the polypeptide has the sequence of SEQ ID NO: 304. |
| 135 | The polypeptide of row 123, wherein the polypeptide has the sequence of SEQ ID NO: 305. |
| 136 | The polypeptide of row 123, wherein the polypeptide has the sequence of SEQ ID NO: 309. |
| 137 | The polypeptide of row 123, wherein the polypeptide has the sequence of SEQ ID NO: 310. |
| 138 | The polypeptide of row 123, wherein the polypeptide has the sequence of SEQ ID NO: 323. |
| 139 | The polypeptide of row 123, wherein the polypeptide has the sequence of SEQ ID NO: 324. |
| 140 | The polypeptide of row 123, wherein the polypeptide has the sequence of SEQ ID NO: 325. |
| 141 | The polypeptide of row 123, wherein the polypeptide has the sequence of SEQ ID NO: 326. |
| 142 | The polypeptide of row 123, wherein the polypeptide has the sequence of SEQ ID NO: 327. |
| 143 | The polypeptide of row 123, wherein the polypeptide has the sequence of SEQ ID NO: 328. |
| 144 | The polypeptide of row 123, wherein the polypeptide has the sequence of SEQ ID NO: 331. |
| 145 | The polypeptide of row 123, wherein the polypeptide has the sequence of SEQ ID NO: 332. |
| 146 | The polypeptide of row 123, wherein the polypeptide has the sequence of SEQ ID NO: 335. |
| 147 | The polypeptide of row 123, wherein the polypeptide has the sequence of SEQ ID NO: 336. |
| 148 | The polypeptide of row 123, wherein the polypeptide has the sequence of SEQ ID NO: 338. |
| 149 | The polypeptide of row 123, wherein the polypeptide has the sequence of SEQ ID NO: 339. |
| 150 | The polypeptide of row 123, wherein the polypeptide has the sequence of SEQ ID NO: 340. |
| 151 | The polypeptide of row 123, wherein the polypeptide has the sequence of SEQ ID NO: 341. |
| 152 | The polypeptide of row 123, wherein the polypeptide has the sequence of SEQ ID NO: 342. |
| 153 | The polypeptide of row 123, wherein the polypeptide has the sequence of SEQ ID NO: 343. |
| 154 | The polypeptide of row 123, wherein the polypeptide has the sequence of SEQ ID NO: 344. |
| 155 | The polypeptide of row 123, wherein the polypeptide has the sequence of SEQ ID NO: 345. |
| 156 | The polypeptide of row 123, wherein the polypeptide has the sequence of SEQ ID NO: 346. |
| 157 | The polypeptide of row 123, wherein the polypeptide has the sequence of SEQ ID NO: 347. |
| 158 | The polypeptide of row 123, wherein the polypeptide has the sequence of SEQ ID NO: 348. |
| 159 | The polypeptide of row 123, wherein the polypeptide has the sequence of SEQ ID NO: 350. |
| 160 | The polypeptide of row 123, wherein the polypeptide has the sequence of SEQ ID NO: 353. |
| 161 | The polypeptide of row 123, wherein the polypeptide has the sequence of SEQ ID NO: 356. |
| 162 | The polypeptide of row 123, wherein the polypeptide has the sequence of SEQ ID NO: 358. |
| 163 | The polypeptide of row 123, wherein the polypeptide has the sequence of SEQ ID NO: 361. |
| 164 | The polypeptide of row 123, wherein the polypeptide has the sequence of SEQ ID NO: 363. |
| 165 | The polypeptide of row 123, wherein the polypeptide has the sequence of SEQ ID NO: 367. |
| 166 | The polypeptide of row 123, wherein the polypeptide has the sequence of SEQ ID NO: 369. |
| 167 | The polypeptide of row 123, wherein the polypeptide has the sequence of SEQ ID NO: 373. |
| 168 | The polypeptide of any one of rows 118-167, wherein the polypeptide forms a dimer. |
| 169 | The polypeptide of row 168, wherein the polypeptide forms a homodimer. |
| 170 | The polypeptide of any one of rows 118-169, wherein the linker is an amino acid spacer. |
| 171 | The polypeptide of row 170, wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 172 | The polypeptide of row 170, wherein the amino acid spacer is GGS, GGGS (SEQ ID NO: 99), |
| | GGGGS (SEQ ID NO: 102), GGSG (SEQ ID NO: 105), or SGGG (SEQ ID NO: 107). |
| 173 | The polypeptide of row 170, wherein the amino acid spacer is GA, GS, GG, GGA, GGS, GGGS |
| | (SEQ ID NO: 99), GGGGA (SEQ ID NO: 101), GGGGS (SEQ ID NO: 102), GGGGG (SEQ ID |
| | NO: 103), GGAG (SEQ ID NO: 104), GGSG (SEQ ID NO: 105), AGGG (SEQ ID NO: 106), |
| | SGGG (SEQ ID NO: 107), GAGA (SEQ ID NO: 108), GSGS (SEQ ID NO: 109), GAGAGA (SEQ |
| | ID NO: 110), GSGSGS (SEQ ID NO: 111), GAGAGAGA (SEQ ID NO: 112), GSGSGSGS (SEQ |
| | ID NO: 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), GGSGGSGGSGGS |
| | (SEQ ID NO: 123), GGAGGGAG (SEQ ID NO: 124), GGSGGGSG (SEQ ID NO: 125), |
| | GGAGGGAGGGAG (SEQ ID NO: 126), and GGSGGGSGGGSG (SEQ ID NO: 127), |
| | GGGGAGGGGAGGGGA (SEQ ID NO: 128), GGGGSGGGGSGGGGS (SEQ ID NO: 129), AAAL |
| | (SEQ ID NO: 133), AAAK (SEQ ID NO: 134), AAAR (SEQ ID NO: 135), EGKSSGSGSESKST |
| | (SEQ ID NO: 136), GSAGSAAGSGEF (SEQ ID NO: 137), AEAAAKEAAAKA (SEQ ID NO: 138), |
| | KESGSVSSEQLAQFRSLD (SEQ ID NO: 139), GENLYFQSGG (SEQ ID NO: 140), SACYCELS |
| | (SEQ ID NO: 141), RSIAT (SEQ ID NO: 142), RPACKIPNDLKQKVMNH (SEQ ID NO: 143), |
| | GGSAGGSGSGSSGGSSGASGTGTAGGTGSGSGTGSG (SEQ ID NO: 144), |
| | AAANSSIDLISVPVDSR (SEQ ID NO: 145), |
| | GGSGGGSEGGGSEGGGSEGGGSEGGGSEGGGSGGGS (SEQ ID NO: 146), EAAAK (SEQ ID |
| | NO: 147), or PAPAP(SEQ ID NO: 148). |
| 174 | The polypeptide of any one of rows 1-173, wherein the polypeptide (e.g., an ActRII chimera-Fc |
| | fusion protein) has a serum half-life of at least seven days. |
| 175 | The polypeptide of any one of rows 1-174, wherein the polypeptide has increased binding to one |
| | or more an ActRII ligands (e.g., activin A, activin B, myostatin, and/or GDF-11) compared to |
| | wild-type ActRIIA and/or wild-type ActRIIB (e.g., wild-type extracellular ActRIIA and/or |
| | ActRIIB). |
| 176 | The polypeptide of any one of rows 1-175, wherein the polypeptide has decreased binding to |
| | bone morphogenetic protein 9 (BMP9, e.g., human BMP9) compared to wild-type ActRIIB (e.g., |
| | wild-type extracellular ActRIIB). |
| 177 | The polypeptide of any one of rows 1-176, wherein the polypeptide binds to activin A, activin B, |
| | and/or myostatin and has reduced or weak binding to human BMP9 (e.g., compared to wild-type |
| | extracellular ActRIIB). |
| 178 | The polypeptide of any one of rows 1-177, wherein the polypeptide does not substantially bind to |
| | human BMP9. |
| 179 | The polypeptide of any one of rows 1-178, wherein the polypeptide binds to human activin A with |
| | a K_{D} of 800 pM or less. |
| 180 | The polypeptide of any one of rows 1-179, wherein the polypeptide binds to human activin B with |
| | a K_{D} of 800 pM or less. |
| 181 | The polypeptide of any one of rows 1-180, wherein the polypeptide binds to human GDF-11 with |
| | a K_{D} of 5 pM or higher. |
| 182 | A nucleic acid molecule encoding the polypeptide of any one of rows 1-181. |
| 183 | A vector comprising the nucleic acid molecule of row 182. |
| 184 | A host cell that expresses the polypeptide of any one of rows 1-181, wherein the host cell |
| | comprises the nucleic acid molecule of row 182 or the vector of row 183, wherein the nucleic |
| | acid molecule or vector is expressed in the host cell. |
| 185 | A pharmaceutical composition comprising the polypeptide of any one of rows 1-181, the nucleic |
| | acid molecule of row 182, or the vector of row 183 and one or more pharmaceutically acceptable |
| | carriers or excipients. |
| 186 | The pharmaceutical composition of row 185, wherein the polypeptide, nucleic acid molecule, or |
| | vector is in a therapeutically effective amount. |
| 187 | A construct comprising two identical polypeptides (e.g., a homodimer), each comprising an |
| | extracellular ActRII chimera of any one of rows 1-117 (e.g., an ActRII chimera having a sequence |
| | of any one of SEQ ID NOs: 174-216 and 221-293) fused (e.g., linked using an amino acid spacer) |
| | to the N- or C-terminus of an Fc domain monomer (e.g., the sequence of SEQ ID NO: 97, SEQ ID |
| | NO: 150, or SEQ ID NO: 155). The two Fc domain monomers in the two polypeptides interact to |
| | form an Fc domain in the construct. |
| 188 | A construct comprising two different polypeptides (e.g., a heterodimer), each comprising an |
| | extracellular ActRII chimera of any one of rows 1-117 (e.g., an ActRII chimera having a sequence |
| | of any one of SEQ ID NOs: 174-216 and 221-293) fused (e.g., linked using an amino acid spacer) |
| | to the N- or C-terminus of an Fc domain monomer (e.g., the sequence of SEQ ID NO: 97, SEQ ID |
| | NO: 150, or SEQ ID NO: 155). The two Fc domain monomers in the two polypeptides interact to |
| | form an Fc domain in the construct. |

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Identification of Altered Serum Biomarkers in Human Subjects Treated with ActRIIB 2.12-Fc

Healthy postmenopausal women were enrolled in a randomized, double-blind, placebo-controlled, two-part study to assess the safety, tolerability, and pharmacokinetics of ActRIIB 2.12-Fc (a homodimer of the polypeptide of SEQ ID NO: 172). Inclusion criteria included being between 45 and 70 years of age, serum FSH>40 IU/L, and a BMI>18.5 kg/m² to <32.0 kg/m², and exclusion criteria included a history of or past treatment for osteoporosis and systemic hormone replacement therapy within three months of the study. In addition to safety, tolerability, and pharmacokinetics, serum biomarkers were also assessed. Described below are results from analysis of serum samples from healthy postmenopausal women administered one subcutaneous dose of either ActRIIB 2.12-Fc at a dose of 4.5 mg/kg or placebo.

Blood samples were collected in SST™ II advance (gold cap, BD Cat #367956) vacutainers, mixed by inverting six times, and placed upright at ambient temperature for a minimum of 30 minutes to a maximum of 60 minutes to allow for clot formation. Samples were centrifuged at 20° C. for 10 mins at 1,300-2999 g (RCF). The supernatant serum was aspirated, aliquoted into microtubes, and stored at −80° C. until used for analysis.

Serum biomarkers were assessed in participants who received a placebo (n=5) on day 1 (prior to treatment administration, considered baseline) and day 15, and in participants who received ActRIIB 2.12-Fc at 4.5 mg/kg (n=6) on day 1 (prior to treatment administration, considered baseline), day 7, day 15, day 22, and day 29.

All samples were measured with the SOMASCAN® platform (SOMASCAN® Proteomics) (www.somalogic.com) (SOMALOGIC®, Boulder, CO) containing 7,596 aptamers (6,408 human protein analytes) that provides measurements of the relative binding of the serum sample to each of the aptamers in relative fluorescence units (RFU). Calibration and normalization samples were used following the manufacturer's recommended protocol. Data standardization was performed according to the SOMASCAN® platform data quality-control protocols. To standardize SOMASCAN® assay results, raw SOMASCAN® assay data were first normalized to remove hybridization variation within a run (hybridization normalization), followed by median signal normalization across all samples to remove other assay biases within the run.

### SOMASCAN® Data Analysis

Multiplex assay: Fifty-five samples were submitted to SomaLogic, LLC, to measure 6,408 proteins by the SOMASCAN® assay. For this analysis, SOMAMER® reagents (aptamers) labeled with a photocleavable linker and biotin were immobilized on streptavidin-coated beads. Each serum sample was incubated with reagents, and serum proteins were allowed to bind, forming SOMAMER®-target protein complexes. Unbound proteins were washed away, and bound proteins were photocleaved with UV light, leaving only the reagents representing the once-bound proteins. The reagents were then bound to complementary sequences of DNA hybridization probes on a microarray. Probes were then quantified by fluorescence. The result was measured as relative fluorescent units (RFU) and is directly proportional to the amount of target protein in the original serum sample.

The SOMASCAN® assay results were normalized to correct systematic effects introduced during the DNA hybridization step. A control sequence introduced into the assay before hybridization was used to calculate a scaling factor, and each sample was normalized to this factor. Additionally, median normalization allows for comparing signals across a plate by correcting for introduced variation from the assay or natural variation in samples' total protein concentrations. Finally, the median signal intensity from each subarray was used to calculate a sample-based scaling factor.

### Statistical Analysis

Quality control assessment and exploratory data analysis were performed for all samples in this study. Hybridization control normalization, intraplate median signal normalization, median signal normalization to a human serum reference, and plate scaling and calibration were performed by SomaLogic for this data. All samples passed the SomaLogic initial quality control assessments for normalization and calibration.

The R package “readat” (Cotton et al., 2016, BMC Bioinformatics) was used to read the SOMASCAN® intensity data. Quality control assessment of proteome profiles was performed using four automated outlier tests, in addition to manual inspection of MA and density plots. No samples failed other automated outlier tests.

Baseline normalization was conducted by combining placebo day 1 and day 15 samples, and ActRIIB 2.12-Fc day 1 samples to generate a baseline group. We then compared this baseline group to either ActRIIB 2.12-Fc day 7, day 15, day 22, and day 29 combined. Models were fit in R with the limma package and logFC extracted for the placebo versus drug comparison.

### NT-Pro BNP ELISA

The serum samples were submitted for analysis of NT-pro BNP via ELISA using a custom proprietary assay developed by Rules Based Medicine (RBM) and conducted at RBM 3300 Duval Road, Suite 110, Austin, TX 78759. This assay was run using Luminex xMAP® technology per RBM house protocol.

### Results

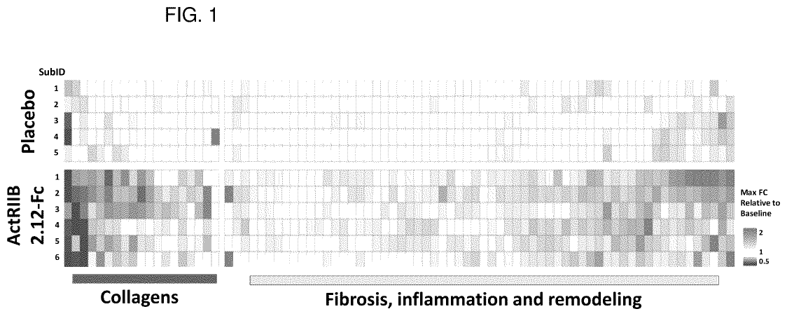

Of the 6,408 proteins in the SOMASCAN® panel, 81 proteins were significantly differentially expressed from baseline in post-menopausal women treated with ActRIIB 2.12-Fc at 4.5 mg/kg versus placebo, with an FDR threshold of 0.01. Of these 81 proteins, 63 were upregulated, and 18 were downregulated vs placebo (FIG. 1).

A clustering analysis was performed on the 81 differentially expressed serum proteins, which found that these proteins have known functions related to inflammation and extracellular matrix remodeling pathways, suggesting that ActRIIB 2.12-Fc has the ability to alter structural remodeling pathways and inflammation, which is consistent with anti-fibrotic activity.

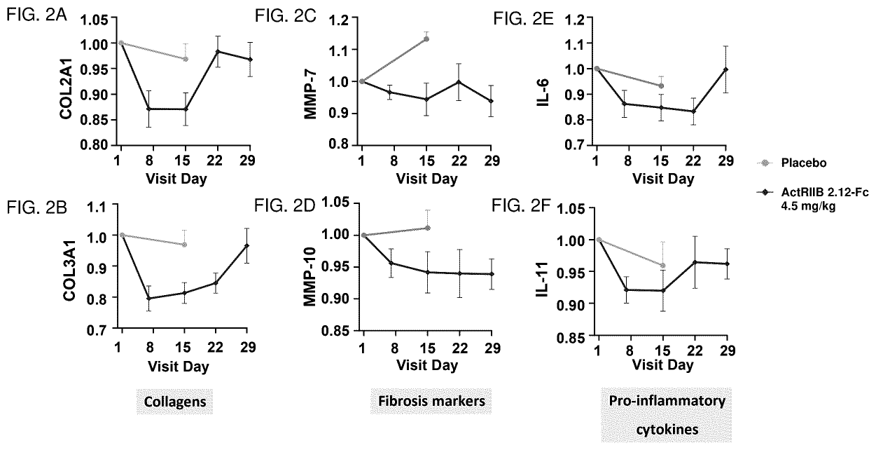

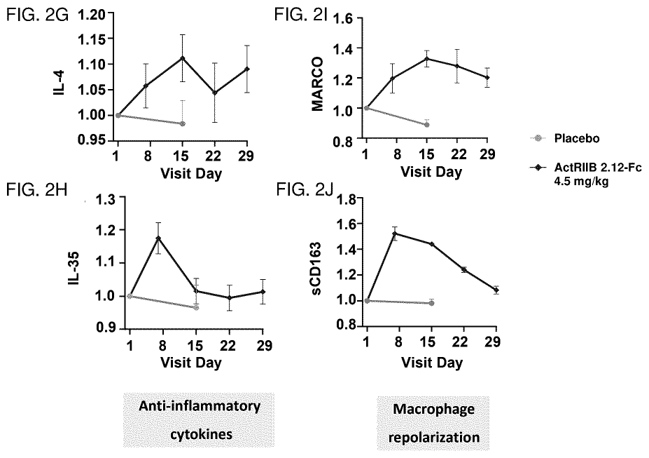

FIGS. 2A-2J show examples of proteins involved in extracellular matrix remodeling and inflammation. Fibrosis markers were decreased, as indicated by changes in matrix metalloproteinases (MMP-7 and MMP-10) and collagen fragments (FIGS. 2A-2D). In addition, a reduction in pro-inflammatory cytokines IL-6 and IL-11 was observed (FIGS. 2E-2F). An increase in anti-inflammatory cytokines (IL-4 and IL-35), and markers of macrophage polarization (MARCO and sCD163) were also observed (FIGS. 2G-2J).

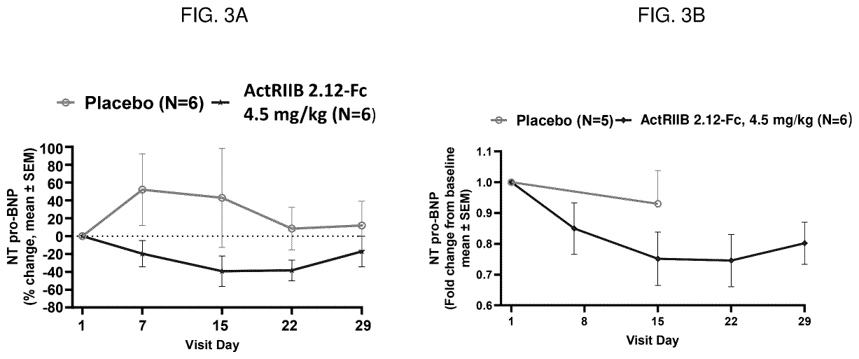

Differentially expressed NT pro-BNP identified by SOMASCAN® (FIG. 3B) was confirmed by measuring the same analyte by ELISA (FIG. 3A). SOMASCAN® data are shown in RFUs and immunoassay data are shown in concentrations. ActRIIB 2.12-Fc administration led to sustained reductions in serum NT pro-BNP, a biomarker of cardiac dysfunction.

### Example 2—ActRIIB 2.12-mFc Attenuated Cardiac Remodeling and Fibrosis in a Transverse Aortic Constriction Model of Heart Failure

Eight-week-old C57BI/6 male mice were subjected to either sham or transverse aortic constriction (TAC) surgery. Sham mice were treated with vehicle (TBS; n=12/grp). TAC mice (n=15/grp) were treated with either vehicle (TAC-vehicle) or ActRIIB 2.12-mFc (a homodimer of SEQ ID NO: 171 linked to a mouse Fc domain monomer by a GGG linker) (10 mg/kg) intraperitoneally (IP) twice weekly starting one (ActRIIB 2.12-mFc Preventative) or 14 (ActRIIB 2.12-mFc Treatment) days after surgery. Eight weeks post-surgery, animals were assessed for associated cardiac pathologies.

In vivo cardiac function was assessed by transthoracic echocardiography (GE NextGen LOGIQ™ e, 22 MHz linear transducer) in lightly anesthetized mice by isoflurane. From left ventricle short axis view, M-mode echocardiogram was acquired to measure interventricular septal thickness at end diastole (IVSd). Early diastolic filling peak velocity (E′) and late filling peak velocity (A′) was measured from the medial or septal wall at the mitral valve level from tissue Doppler image. E and A peak velocity was measured from pulse Doppler image. LV diastolic function was assessed by measuring the E/E′ ratio. Three to five beats were averaged for each mouse study. Studies and analysis were performed by investigators blinded to treatments.

Fibrosis was quantified from fixed lung tissue using computerized planimetry (Image J, NIH). Mice were assessed terminally for markers of tissue remodeling in the left ventricle (LV) by reverse transcription quantitative polymerase chain reaction (RT-qPCR).

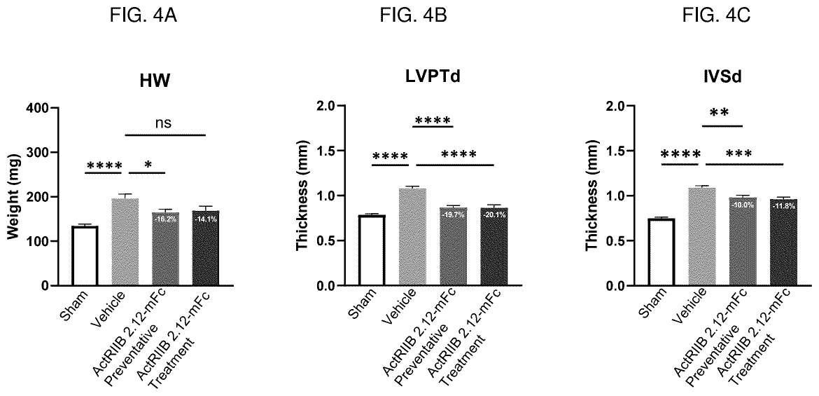

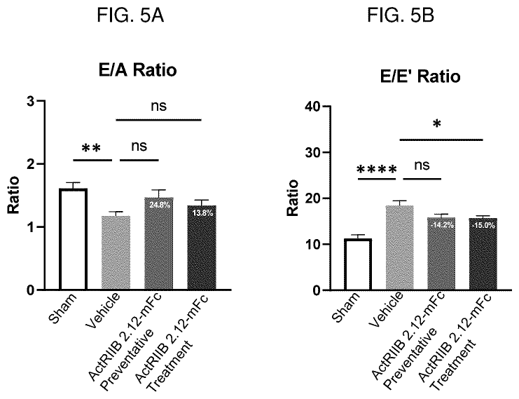

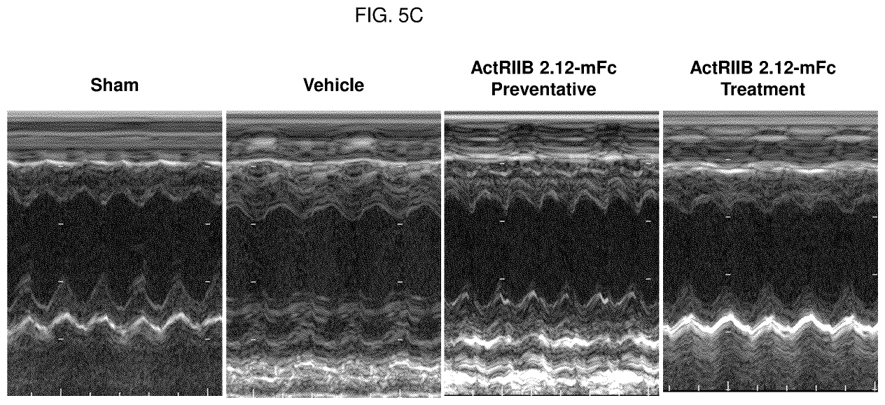

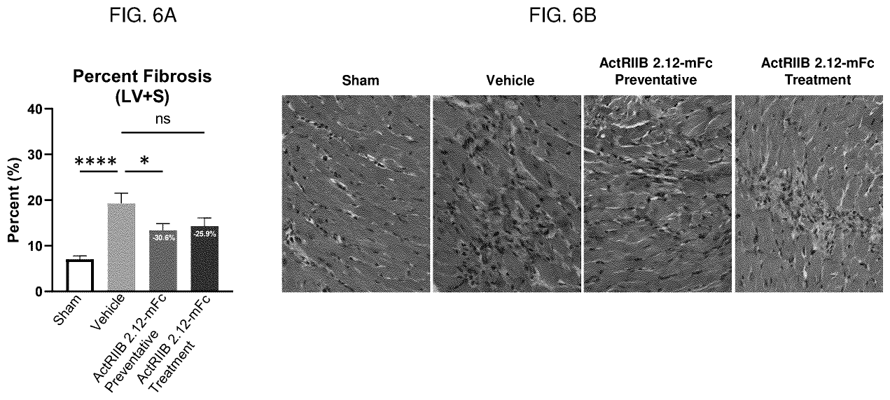

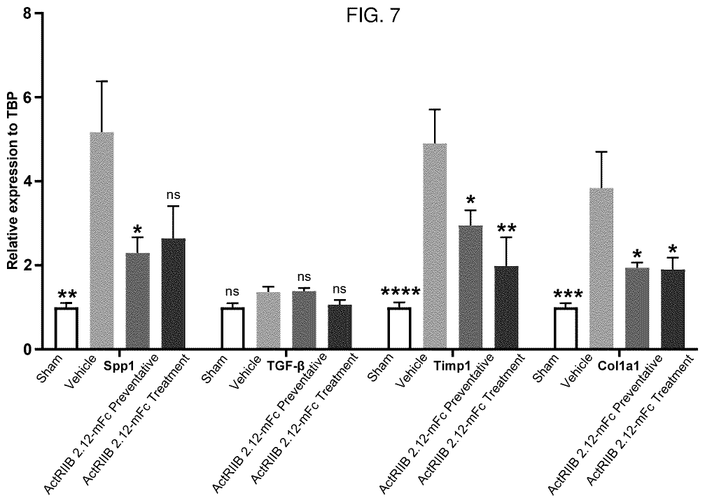

Results are shown in FIGS. 4A-4C, 5A-5C, 6A-6B, and 7. All data represented as mean±SEM. Data analyzed using one-way ANOVA followed by Dunnett post hoc test. *p≤0.05; **p≤0.01; ***p≤0.001; ****p≤0.0001; ns—not significant. Percent change compared to TAC-Vehicle.

### Example 3—ActRIIa/b-mFc Attenuated Cardiac Remodeling in a Transverse Aortic Constriction Model of Heart Failure

Eight-week-old C57BI/6 male mice were subjected to either sham or transverse aortic constriction (TAC) surgery and left for 28 days for pathology to progress. Twenty-eight days after surgery, one group of mice (n=9) was treated with vehicle (TAC-vehicle) and the other (n=11) treated with ActRIIa/b-mFc (a homodimer of SEQ ID NO: 69 linked to a mouse Fc domain monomer by a GGG linker) (20 mg/kg) intraperitoneally (IP) twice weekly. Eight weeks post-surgery, animals were assessed by transthoracic echocardiograph imaging.

In vivo cardiac function was assessed by transthoracic echocardiography (FUJIFILM Visual Sonics Vevo® 3100 with a 24 MHz MX Series transducer) in mice lightly anesthetized with isoflurane. From this, ejection fraction, fractional shortening, left ventricle mass, and left ventricle volume during diastole and during systole were determined.

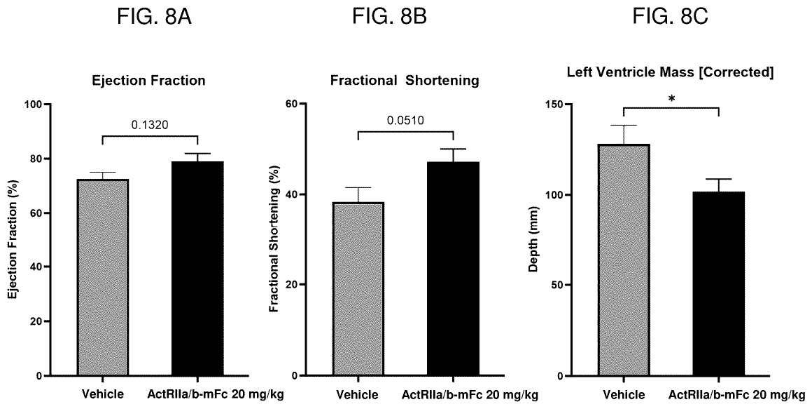

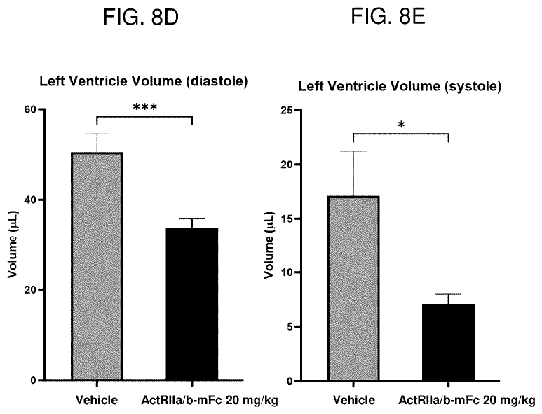

Results are shown in FIGS. 8A-8E. All data represented as mean±SEM. Data analyzed using a student's T Test. *p≤0.05; **p≤0.01; ***p≤0.001; ****p≤0.0001; ns—not significant. Percent change compared to TAC-Vehicle.

### Example 4—Effect of ActRIIA/B-hFc on NT-proBNP in Patients with Very Low, Low, or Intermediate Risk Myelodysplastic Syndromes

Patients with IPSS-R very-low, low, or intermediate risk MDS and anemia were enrolled in a multi-center, open-label, two-part, Phase 2 study. In Part 2, participants received the recommended Part 2 dose (RP2D; 3.75 mg/kg ActRIIA/B-hFc (a homodimer of the polypeptide of SEQ ID NO: 156)) administered subcutaneously once every 4 weeks), deemed the MDS RP2D population, with up-titration allowed per protocol to 5 mg/kg once every 4 weeks. Primary and secondary endpoints assess safety and tolerability, rates of hematologic response (modified IWG 2006 hematologic improvement-erythroid (HI-E) and red blood cell transfusion independence (RBC-TI)), and changes in hemoglobin (Hgb). Exploratory endpoints include evaluation of the following biomarkers:
Iron metabolism: serum ferritin, hepcidin
Erythropoiesis: Hgb and soluble transferrin receptor (sTfR; a marker of erythropoietic activity)
Thrombopoiesis: platelet count
Bone turnover: bone specific alkaline phosphatase (BSAP; a marker of osteoblast activity)
Cardiac function: N-terminal prohormone of brain natriuretic protein (NT-proBNP; a marker of myocardial strain)

### Baseline Characteristics

At the time of the assessment, most participants had multilineage dysplasia (MLD) and were transfusion dependent. Approximately 56% of participants had high transfusion burden (HTB) and nearly a third were heavily transfused (≥6 RBC units/8 weeks). Approximately 20% of participants had intermediate-risk disease based on IPSS-R; recategorization by IPSS-M rendered a higher proportion (nearly 28%) in the corresponding Moderate Low and Moderate High IPSS-M categories, and 6 participants were recategorized as IPSS-M High or Very High risk. Baseline characteristics for the MDS RP2D population are provided in Table 13 below.

TABLE 13
Baseline characteristics in MDS RP2D population

| Parameter | RP2D (N = 79) | |
|---|---|---|
| Median Age, years (range) | 73.4 | (53-89) |
| Sex, n (%) male | 50 | (63.3) |
| RBC transfusion status, units per 8 weeks, n (%) | | |
| Non-transfused (NT), 0 units | 15 | (19.0) |
| Low transfusion burden (LTB), <4 units | 20 | (25.3) |
| ≥2 to <4 units | 17 | (21.5) |
| High transfusion burden (HTB), ≥4 units | 44 | (55.7) |
| ≥6 units | 25 | (31.6) |
| Hemoglobin, g/dL, median (range) | 8.37 | (3.7-10.5) |
| Ring sideroblasts Status, n (%) | | |
| RS+ | 57 | (72.2) |
| Non-RS | 22 | (27.8) |
| IPSS-R risk category, n (%) | | |
| Very low/Low | 62 | (78.5) |
| Intermediate | 16 | (20.3) |
| Missing^{#} | 1 | (1.3) |
| IPSS-M Risk Category^{‡}, n (%) | | |
| Very low/Low | 49 | (62.0) |
| Moderate low | 13 | (16.5) |
| Moderate high | 9 | (11.4) |
| High/Very high | 6 | (7.6) |
| Missing | 2 | (2.5) |
| Dysplasia category, n (%) | | |
| Multi-lineage (MLD) | 50 | (63.3) |
| Single-lineage (SLD) | 7 | (8.9) |
| Unknown/Missing | 22 | (27.8) |
| Prior ESA, n (%) | 21 | (26.6) |
| EPO ≥200 U/L, n (%) | 26 | (32.9) |
| ≥500 U/L, n (%) | 12 | (15.2) |
| Iron chelator therapy | 21 | (26.6) |

ESA = erythroid stimulating agent; EPO = erythropoietin; IPSS-R/M = international prognostic scoring system-revised/molecular; MDS = myelodysplastic syndromes; RBC = red blood cell.
^{#}IPSS-R missing is due to unavailable cytogenetic data as of data cut-off date.
^{‡}IPSS-M derivation used a “best case” approach to handle missing data for prognostic genes, imputing prognostic mutations as “not detected” if no data were available.

### Baseline Biomarkers

Levels of sTfR were generally normal despite anemia. Ferritin levels were generally elevated while hepcidin levels were within the normal range. Median platelet counts and BSAP levels were within the normal range. Several participants had elevated NT-proBNP. Baseline biomarkers are provided in Table 14 below.

TABLE 14
Baseline biomarkers

| | |
|---|---|
| Parameter, median (range) | RP2D (N = 79) |

Hematopoiesis

| | | |
|---|---|---|
| sTfR, mg/L | 1.59 | (0.2-7.1) |
| CHr, pg/cell | 34.70 | (27.4-43.8) |
| MCH, pg/cell | 33.0 | (26-41) |

Iron

| | | |
|---|---|---|
| Hepcidin, ng/mL, N = 49* | 22.31 | (2.8-106.0) |
| Ferritin, ng/mL | 821.99 | (86.3-5829.1) |

Other

| | | |
|---|---|---|
| Platelets, ×10⁹/L | 216.0 | (37-441) |
| BSAP, ug/L | 12.75 | (6.2-60.7) |
| NT-proBNP, pg/mL, N = 15* | 300.85 | 50.0-1118.6) |

BSAP = bone specific alkaline phosphatase; CHr = reticulocyte hemoglobin content (LLN = 24, ULN = 36); MCH = mean corpuscular hemoglobin; NT-proBNP = N terminal-prohormone of brain natriuretic protein; sTfR = soluble transferrin receptor.
Reference ranges (lower limit of normal [LLN], upper limit of normal [ULN]): sTfR: (0.76, 1.76), CHr (24, 36) MCH (26, 34), hepcidin (2.8, 41.8), ferritin (10, 322) platelets (150, 450), BSAP (5.6, 29), NT-proBNP (<450 pg/mL 75 years or older).
*Smaller sample size for hepcidin is because analysis only included baseline and postbaseline data that used the same assay; smaller sample size for NT-proBNP is due to recent addition into protocol leading to limited availability of baseline samples as of data cut-off date

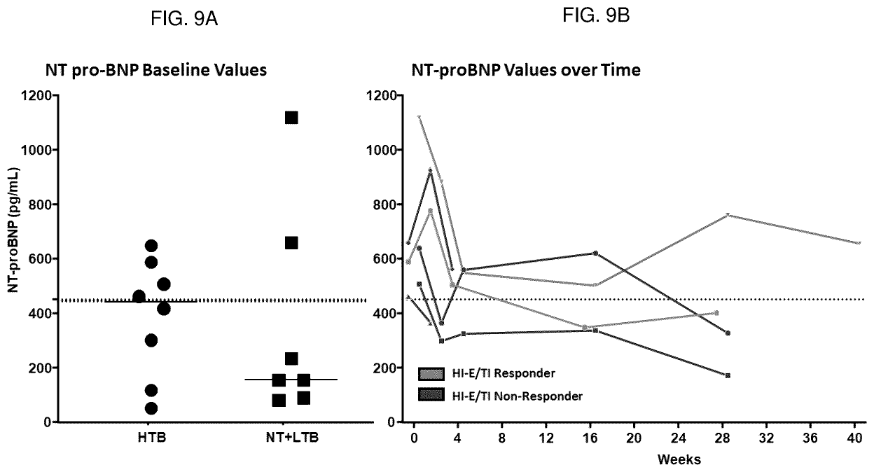

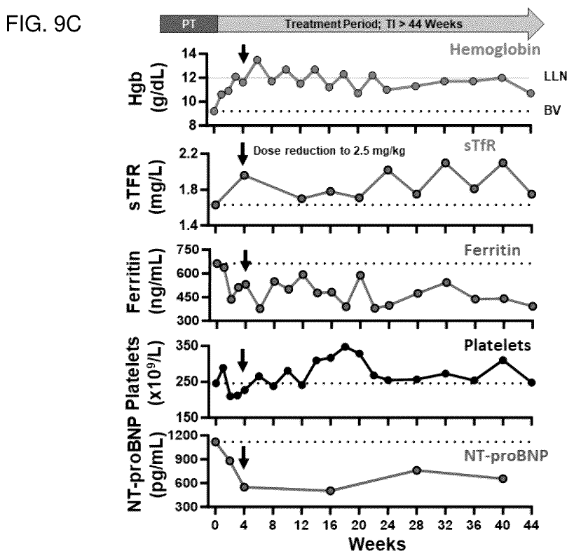

In patients with lower-risk MDS, cardiovascular (CV) events represent a major cause of death possibly due to myocardial stress exacerbated by chronic anemia and cardiac iron overload. NT-proBNP is a marker of myocardial stress, and elevated levels have been associated with increased risk for CV mortality. In a limited data set, six MDS RP2D participants were identified with elevated (≥450 pg/mL) levels of NT-proBNP at baseline (FIG. 9A). While most had HTB, the highest baseline levels were observed among two participants with LTB. Among these six participants with elevated NT-proBNP levels at baseline, decreases were observed with ActRIIA/B-hFc treatment in both hematological responders and non-responders (FIG. 9B). Collectively, these data suggest that ActRIIA/B-hFc may ameliorate cardiac strain both directly (via activin A inhibition) and indirectly (through improving anemia and reducing transfusion burden). A transfusion-dependent (LTB) participant (FIG. 9C; 81-year-old female with MDS-RS-MLD) was observed to experience improvements in several biomarkers relevant to MDS:
Improved erythropoiesis as demonstrated by sustained increases in Hgb with increased sTfR, resulting in TI>44 weeks that was ongoing as of the data cut-off date.
Substantial and sustained decreases in ferritin ≥20% suggesting reduced 10.
Stable to improved platelet counts indicative of improved multilineage hematopoiesis.
Precipitous decrease in NT-proBNP after a single dose of ActRIIA/B-hFc that was generally maintained while on treatment.

In view of these data, ActRIIA/B-hFc may ameliorate myocardial stress in subjects with MDS, potentially both via direct beneficial effects on the myocardium and indirect effects by reducing transfusions and 10, as demonstrated by decreases in NT-proBNP observed in participants with high baseline levels, irrespective of an erythroid response.

### Example 5—Effect of ActRIIA/B-hFc on NT-proBNP in Patients with Very Low, Low, or Intermediate Risk Myelodysplastic Syndromes

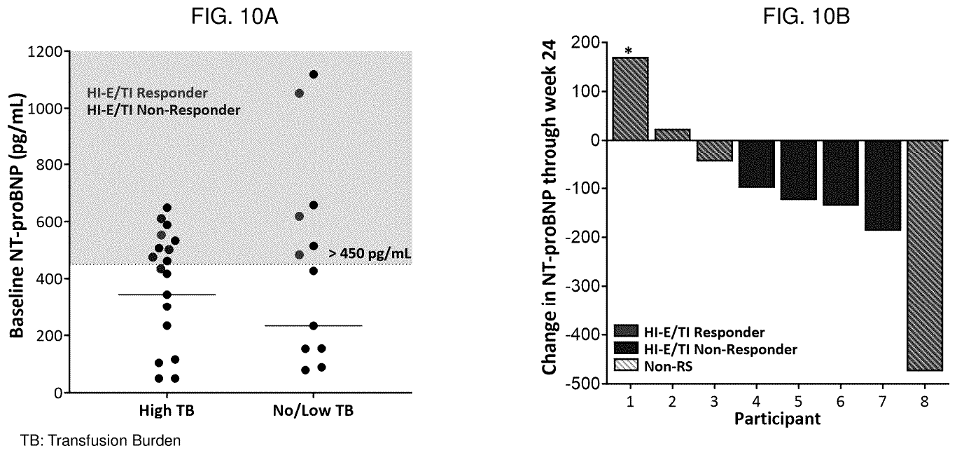

A second assessment of NT-proBNP levels was performed on the subjects from the study described in Example 4 once additional participants had been enrolled and study duration had increased. NT-proBNP, an independent risk factor for heart failure and all-cause mortality, was analyzed in limited serum samples from subjects participating in the trial. Eight trial participants with LR-MDS had baseline NT-proBNP >450 pg/mL, levels which have been associated with increased risk of CV events (FIG. 10A). Three of these subjects had LTB (no transfusions or <4 units transfused per 8 weeks) and were HI-E/TI responders, one subject had HTB (≥4 units transfused per 8 weeks) and was an HI-E/TI responder, and three subjects had HTB and were HI-E/TI non-responders. ActRIIA/B-hFc reduced NT-proBNP serum levels in six out of eight participants with elevated NT-proBNP levels, regardless of erythroid response (FIG. 10B). One patient, denoted with an asterisk in FIG. 10B, had a medical history including atrial fibrillation, ablation atrial fibrillation, chronic heart failure, ischemic heart disease, and thrombocytopenia and adverse events of worsening of ischemic heart disease and worsening of chronic heart failure. Data are presented for participants who received the recommended Part 2 dose of ActRIIA/B-hFc (3.75 mg/kg every 4 weeks) with titration allowed to 5 mg/kg every 4 weeks. That the observed reductions in NT-proBNP after treatment with ActRIIA/B-hFc occurred in participants with LR-MDS regardless of erythropoietic response supports the potential for direct benefit of ActRIIA/B-hFc on the myocardium and cardiac health via activin A inhibition.

### Example 6—Treatment of a Cardiovascular Disease by Administration of an Extracellular ActRII Variant

According to the methods disclosed herein, a physician of skill in the art can treat a subject, such as a human patient, having or at risk of developing a cardiovascular disease (e.g., left-sided heart failure (e.g., heart failure with reduced ejection fraction (HFrEF) or heart failure with preserved ejection fraction (HFpEF), such as chronic HFrEF or chronic HFpEF), right-sided heart failure, backward heart failure, forward heart failure, high-output heart failure, low-output heart failure, compensated heart failure, or decompensated heart failure, cardiomyopathy, a calcification disease, heart valve disease, vasculitis, an arrythmia, ventricular dysfunction, or an aneurysm) so as to prevent the cardiovascular disease, delay the development of the cardiovascular disease, slow the progression of the cardiovascular disease, or reduce cardiac remodeling, fibrosis, or inflammation associated with the cardiovascular disease. To treat the subject, a physician of skill in the art can administer to the subject a composition containing a polypeptide including an extracellular ActRII variant (e.g., a polypeptide containing an extracellular ActRIIA variant having the sequence of any one of SEQ ID NOs: 1-72 (e.g., SEQ ID NOs: 6-72), a polypeptide containing an extracellular ActRIIB variant having the sequence of any one of SEQ ID NOs: 157-171 (e.g., SEQ ID NOs: 158-171), or a polypeptide containing an extracellular ActRII chimera having the sequence of any one of SEQ ID NOs: 174-216 (e.g., SEQ ID NOs: 195-216), such as a polypeptide containing an extracellular ActRIIA variant, an extracellular ActRIIB variant, or an extracellular ActRII chimera linked to an Fc domain monomer (e.g., a polypeptide that is in the form of a homodimer of an extracellular ActRIIA variant, an extracellular ActRIIB variant, or an extracellular ActRII chimera linked to an Fc domain monomer, which forms an Fc domain in the dimer)). The composition containing the extracellular ActRII variant may be administered to the subject, for example, by parenteral injection (e.g., intravenous or subcutaneous injection) to treat the cardiovascular disease. The polypeptide containing an extracellular ActRII variant is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII variant is administered once every sixteen weeks, quarterly, once every twelve weeks, bimonthly, once every eight weeks, once a month, once every four weeks, once every two weeks, or at least once a week or more (e.g., 1, 2, 3, 4, 5, 6, or 7 times a week or more). The extracellular ActRII variant is administered in an amount sufficient to reduce cardiac remodeling, fibrosis, or inflammation associated with the cardiovascular disease, prevent the disease, delay the development of the disease, or slow progression of the disease.

Following administration of the composition to a patient, a practitioner of skill in the art can monitor the patient's improvement in response to the therapy by a variety of methods. For example, a physician can monitor the patient's cardiac function and structure using a variety of tests, such as a blood test, chest x-ray, electrocardiogram, echocardiogram, ejection fraction, exercise test or stress test, cardiac CT scan, cardiac MRI, coronary angiogram, and/or myocardial biopsy. A finding that the patient's cardiac function is improved or stabilized or that the disease is progressing more slowly following administration of the composition compared to test results prior to administration of the composition indicates that the patient is responding favorably to the treatment. Subsequent doses can be determined and administered as needed.

### Example 7—Treatment of a HFrEF or HFpEF by Administration of an Extracellular ActRIIB Variant

According to the methods disclosed herein, a physician of skill in the art can treat a subject, such as a human patient, having HFrEF or HFpEF (e.g., chronic HFpEF or chronic HFrEF with elevated NT-proBNP (>500 pg/mL)) so as to slow or inhibit the progression of HFrEF or HFpEF (e.g., progression of NYHA Functional Class symptoms, such as from NYHA Functional Class II to NYHA Functional Class III, or from NYHA Functional Class Ill to NYHA Functional Class IV), reverse HFrEF or HFpEF (e.g., reverse NYHA Functional Class symptoms, such as from NYHA Functional Class IV to NYHA Functional Class III, from NYHA Functional Class Ill to NYHA Functional Class II, or from NYHA Functional Class II to NYHA Functional Class I), reduce NT-proBNP levels, improve CPET parameters (e.g., respiratory gas exchange, heart rate, blood pressure, or other physiological variables assessed by cardiopulmonary exercise test, such as a test using a cycle ergometer or treadmill), improve ECHO parameters (e.g., LVEF or other cardiac function variables measured by echocardiogram), reduce cardiac remodeling, reduce fibrosis, or reduce inflammation. The subject may have HFpEF with defined ≥Grade 2 diastolic dysfunction and/or stable NYHA Functional Class II to IV symptoms. The subject may also be taking a HF background therapy. To treat the subject, a physician of skill in the art can administer to the subject a composition containing a polypeptide including an extracellular ActRIIB variant (e.g., a polypeptide containing an extracellular ActRIIB variant having the sequence of any one of SEQ ID NOs: 157-171 (e.g., SEQ ID NOs: 158-171), such as a polypeptide of SEQ ID NO: 172, e.g., a homodimer of the polypeptide of SEQ ID NO: 172). The composition containing the extracellular ActRIIB variant may be administered to the subject, for example, by parenteral injection (e.g., intravenous or subcutaneous injection) to treat the HFrEF or HFpEF. The polypeptide containing an extracellular ActRIIB variant is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRIIB variant is administered once every sixteen weeks, quarterly, once every twelve weeks, bimonthly, once every eight weeks, once a month, once every four weeks, once every two weeks, or at least once a week or more (e.g., 1, 2, 3, 4, 5, 6, or 7 times a week or more). In some embodiments, the polypeptide including an extracellular ActRIIB variant (e.g., a polypeptide containing an extracellular ActRIIB variant having the sequence of SEQ ID NO: 171, such as the polypeptide of SEQ ID NO: 172, e.g., a homodimer of the polypeptide of SEQ ID NO: 172) is administered by subcutaneous injection at 4.5 mg/kg once every 28 days (i.e., once every four weeks). The extracellular ActRIIB variant is administered in an amount sufficient to reduce cardiac remodeling, fibrosis, or inflammation associated with HFrEF or HFpEF, reduce NT-proBNP levels, improve CPET parameters, improve ECHO parameters, slow or inhibit progression of HFrEF or HFpEF, or reverse HFrEF or HFpEF.

Following administration of the composition to a patient, a practitioner of skill in the art can monitor the patient's improvement in response to the therapy by a variety of methods. For example, a physician can monitor the patient's cardiac function and structure using a variety of tests, such as a blood test, blood pressure test, chest x-ray, electrocardiogram, echocardiogram, exercise test or stress test, cardiac CT scan, cardiac MRI, coronary angiogram, and/or myocardial biopsy. A finding that the patient's cardiac function is improved or stabilized or that the disease is progressing more slowly following administration of the composition compared to test results prior to administration of the composition indicates that the patient is responding favorably to the treatment. Subsequent doses can be determined and administered as needed.

### Example 8—Reduction of Cardiovascular Risk in a Subject Having a Myelodysplastic Syndrome by Administration of an Extracellular ActRII Variant

According to the methods disclosed herein, a physician of skill in the art can treat a subject, such as a human patient, having a myelodysplastic syndrome (e.g., very low-, low-, or intermediate-risk MDS as determined by IPSS-R, such as a subject having very low-, low-, or intermediate-risk MDS and NT-proBNP≥450 pg/mL) so as to reduce cardiovascular risk (e.g., reduce the risk of developing a cardiovascular disease, reduce the risk or occurrence of cardiovascular events, reduce or inhibit progression of a cardiovascular disease, delay the development or onset of a cardiovascular disease, reduce cardiac strain, improve cardiac function, or prevent or reduce the risk of cardiovascular disease-related mortality). To treat the subject, a physician of skill in the art can administer to the subject a composition containing a polypeptide including an extracellular ActRII variant (e.g., a polypeptide containing an extracellular ActRIIA variant having the sequence of any one of SEQ ID NOs: 1-72 (e.g., SEQ ID NOs: 6-72), a polypeptide containing an extracellular ActRIIB variant having the sequence of any one of SEQ ID NOs: 157-171 (e.g., SEQ ID NOs: 158-171), or a polypeptide containing an extracellular ActRII chimera having the sequence of any one of SEQ ID NOs: 174-216 (e.g., SEQ ID NOs: 195-216), such as a polypeptide containing an extracellular ActRIIa variant, an extracellular ActRIIB variant, or an extracellular ActRII chimera linked to an Fc domain monomer (e.g., a polypeptide that is in the form of a homodimer of an extracellular ActRIIA variant, an extracellular ActRIIB variant, or an extracellular ActRII chimera linked to an Fc domain monomer, which forms an Fc domain in the dimer)). The composition containing the extracellular ActRII variant may be administered to the subject, for example, by parenteral injection (e.g., intravenous or subcutaneous injection). The polypeptide containing an extracellular ActRII variant is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII variant is administered once every sixteen weeks, quarterly, once every twelve weeks, bimonthly, once every eight weeks, once a month, once every four weeks, once every two weeks, or at least once a week or more (e.g., 1, 2, 3, 4, 5, 6, or 7 times a week or more). In some embodiments, the polypeptide including an extracellular ActRII variant is a polypeptide including an extracellular ActRIIA variant (e.g., a polypeptide containing an extracellular ActRIIA variant having the sequence of SEQ ID NO: 69, such as the polypeptide of SEQ ID NO: 156, e.g., a homodimer of the polypeptide of SEQ ID NO: 156) and is administered by subcutaneous injection at 0.75 mg/kg to 5 mg/kg (e.g., 3.75 mg/kg) once every 28 days (i.e., once every four weeks). The extracellular ActRII variant is administered in an amount sufficient to reduce NT-proBNP levels, improve cardiac function, reduce the risk of developing a cardiovascular disease, reduce the risk or occurrence of cardiovascular events, slow or inhibit progression of a cardiovascular disease, delay the development or onset of a cardiovascular disease, reduce cardiac strain, and/or prevent or reduce the risk of cardiovascular disease-related mortality.

Following administration of the composition to a patient, a practitioner of skill in the art can monitor the patient's improvement in response to the therapy by a variety of methods. For example, a physician can monitor the patient's cardiac function and structure using a variety of tests, such as a blood test, blood pressure test, chest x-ray, electrocardiogram, echocardiogram, exercise test or stress test, cardiac CT scan, cardiac MRI, coronary angiogram, and/or myocardial biopsy. A finding that the patient's cardiac function is improved or stabilized or that biomarkers such as NT-proBNP are decreased following administration of the composition compared to test results prior to administration of the composition indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent, or patent application was specifically and individually indicated to be incorporated by reference in its entirety.

Other embodiments are within the following claims.

1. A method of treating a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, hypertriglyceridemia, or ventricular dysfunction, the method comprising administering to the subject a therapeutically effective amount of:
a) a polypeptide comprising an extracellular activin receptor type IIB (ActRIIB) variant, the variant having one or more amino acid substitutions relative to the sequence of GRGEAETRECIYYNANWELERTNQSGLERCEGEQDKRLHCYASWRNSSGTIELVKKGCWLDDFNCYDRQE CVATEENPQVYFCCCEGNFCNERFTHLPEAGGPEVTYEPPPTAPT (SEQ ID NO: 74), wherein the variant comprises one or more amino acid substitutions that impart reduced BMP9 binding relative to wild type extracellular ActRIIB and one or more additional amino acid substitutions, wherein the substitutions that reduce BMP9 binding comprise one or more of:
i) amino acid substitution E75K;
ii) amino acid substitutions Q69T and E70D; or
iii) amino acid substitutions Q69D and E70T,
optionally wherein the variant is truncated from the N-terminus by deletion of 1, 2, 3, 4, 5, 6, or 7 amino acids; or
b) a polypeptide comprising an extracellular activin receptor type II (ActRII) chimera, the chimera having a sequence of any one of
(SEQ ID NO: 174)
GAILGRAETRECIYYNANWELERTNQSGLERCEGEQX₁KRRHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 175)
GAILGRAETRECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 176)
GAILGRAETRECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 177)
GAILGRAETRECIYYNANWELERTNQSGLERCEGEQX₁KRLHCYASWRNSSGSIEIVKQGCWLD
DX₂X₃CYDRTDCVX₄X₅X₆X₇X₈PX₉VYFCCCEGNMCNEKFSYFPEMEVTQPTS,
(SEQ ID NO: 178)
GAILGRAETRECIYYNANWELERTNQSGLERCEGEQX₁KRLHCYASWRNSSGTIEIVKQGCWLD
DX₂X₃CYDRTDCVX₄X₅X₆X₇X₈PX₉VYFCCCEGNMCNEKFSYFPEMEVTQPTS,
(SEQ ID NO: 179)
GAILGRAETRECIYYNANWELERTNQSGLERCEGEQX₁KRLHCYASWRNSSGTIELVKKGCWLD
DX₂X₃CYDRTDCVX₄X₅X₆X₇X₈PX₉VYFCCCEGNMCNEKFSYFPEMEVTQPTS,
(SEQ ID NO: 180)
GAILGRAETRECIYYNANWELERTNQSGLERCEGEQX₁KRLHCYASWRNSSGTIELVKKGCWLD
DX₂X₃CYDRQECVX₄X₅X₆X₇X₈PX₉VYFCCCEGNMCNEKFSYFPEMEVTQPTS,
(SEQ ID NO: 181)
GRGEAETRECIYYNANWELERTNQSGLERCEGEQX₁KRRHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 182)
GRGEAETRECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 183)
GRGEAETRECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 184)
GRGEAETRECIYYNANWELERTNQSGLERCEGEQX₁KRLHCYASWRNSSGSIEIVKQGCWLDD
X₂X₃CYDRTDCVX₄X₅X₆X₇X₈PX₉VYFCCCEGNMCNEKFSYFPEMEVTQPTS,
(SEQ ID NO: 185)
GRGEAETRECIYYNANWELERTNQSGLERCEGEQX₁KRLHCYASWRNSSGTIEIVKQGCWLDD
X₂X₃CYDRTDCVX₄X₅X₆X₇X₈PX₉VYFCCCEGNMCNEKFSYFPEMEVTQPTS,
(SEQ ID NO: 186)
GRGEAETRECIYYNANWELERTNQSGLERCEGEQX₁KRLHCYASWRNSSGTIELVKKGCWLDD
X₂X₃CYDRTDCVX₄X₅X₆X₇X₈PX₉VYFCCCEGNMCNEKFSYFPEMEVTQPTS,
(SEQ ID NO: 187)
GRGEAETRECIYYNANWELERTNQSGLERCEGEQX₁KRLHCYASWRNSSGTIELVKKGCWLDD
X₂X₃CYDRQECVX₄X₅X₆X₇X₈PX₉VYFCCCEGNMCNEKFSYFPEMEVTQPTS,
(SEQ ID NO: 188)
GAILGRSETQECIYYNANWELERTNQSGLERCEGEQX₁KRRHCFATWKNISGSIEIVKQGCWLD
DX₂X₃CYDRTDCVX₄X₅X₆X₇X₈PX₉VYFCCCEGNMCNEKFSYFPEMEVTQPTS,
(SEQ ID NO: 189)
GAILGRSETQECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 190)
GAILGRSETQECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 191)
GAILGRSETQECIYYNANWELERTNQSGLERCEGEQX₁KRLHCYASWRNSSGSIEIVKQGCWLD
DX₂X₃CYDRTDCVX₄X₅X₆X₇X₈PX₉VYFCCCEGNMCNEKFSYFPEMEVTQPTS,
(SEQ ID NO: 192)
GAILGRSETQECIYYNANWELERTNQSGLERCEGEQX₁KRLHCYASWRNSSGTIEIVKQGCWLD
DX₂X₃CYDRTDCVX₄X₅X₆X₇X₈PX₉VYFCCCEGNMCNEKFSYFPEMEVTQPTS,
(SEQ ID NO: 193)
GAILGRSETQECIYYNANWELERTNQSGLERCEGEQX₁KRLHCYASWRNSSGTIELVKKGCWLD
DX₂X₃CYDRTDCVX₄X₅X₆X₇X₈PX₉VYFCCCEGNMCNEKFSYFPEMEVTQPTS,
and
(SEQ ID NO: 194)
GAILGRSETQECIYYNANWELERTNQSGLERCEGEQX₁KRLHCYASWRNSSGTIELVKKGCWLD
DX₂X₃CYDRQECVX₄X₅X₆X₇X₈PX₉VYFCCCEGNMCNEKFSYFPEMEVTQPTS,
wherein X₁ is D or R, X₂ is I, F, E, D, Y, S, N, Q, or T, X₃ is N or T, X₄ is A or E, X₅ is T or K, X₆ is E or K, X₇ is E or D, X₈ is N or S, and X₉ is Q, E, K, R, D, or N, optionally wherein the chimera is truncated from the N-terminus by deletion of 1, 2, 3, 4, 5, 6, 7, 8, or 9 amino acids, wherein the chimera retains the two amino acids before the first cysteine.

2-8. (canceled)

9. A method of reducing inflammation, reducing cardiac remodeling, reducing fibrosis, or improving cardiac or ventricular function in a subject having or at risk of developing a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia, the method comprising administering to the subject a therapeutically effective amount of:
a) a polypeptide comprising an extracellular ActRIIB variant, the variant having one or more amino acid substitutions relative to the sequence of GRGEAETRECIYYNANWELERTNQSGLERCEGEQDKRLHCYASWRNSSGTIELVKKGCWLDDFNCYDRQE CVATEENPQVYFCCCEGNFCNERFTHLPEAGGPEVTYEPPPTAPT (SEQ ID NO: 74), wherein the variant comprises one or more amino acid substitutions that impart reduced BMP9 binding relative to wild type extracellular ActRIIB and one or more additional amino acid substitutions, wherein the substitutions that reduce BMP9 binding comprise one or more of:
i) amino acid substitution E75K;
ii) amino acid substitutions Q69T and E70D; or
iii) amino acid substitutions Q69D and E70T,
optionally wherein the variant is truncated from the N-terminus by deletion of 1, 2, 3, 4, 5, 6, or 7 amino acids; or
b) a polypeptide comprising an extracellular ActRII chimera, the chimera having a sequence of any one of
(SEQ ID NO: 174)
GAILGRAETRECIYYNANWELERTNQSGLERCEGEQX₁KRRHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 175)
GAILGRAETRECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 176)
GAILGRAETRECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 177)
GAILGRAETRECIYYNANWELERTNQSGLERCEGEQX₁KRLHCYASWRNSSGSIEIVKQGCWLD
DX₂X₃CYDRTDCVX₄X₅X₆X₇X₈PX₉VYFCCCEGNMCNEKFSYFPEMEVTQPTS,
(SEQ ID NO: 178)
GAILGRAETRECIYYNANWELERTNQSGLERCEGEQX₁KRLHCYASWRNSSGTIEIVKQGCWLD
DX₂X₃CYDRTDCVX₄X₅X₆X₇X₈PX₉VYFCCCEGNMCNEKFSYFPEMEVTQPTS,
(SEQ ID NO: 179)
GAILGRAETRECIYYNANWELERTNQSGLERCEGEQX₁KRLHCYASWRNSSGTIELVKKGCWLD
DX₂X₃CYDRTDCVX₄X₅X₆X₇X₈PX₉VYFCCCEGNMCNEKFSYFPEMEVTQPTS,
(SEQ ID NO: 180)
GAILGRAETRECIYYNANWELERTNQSGLERCEGEQX₁KRLHCYASWRNSSGTIELVKKGCWLD
DX₂X₃CYDRQECVX₄X₅X₆X₇X₈PX₉VYFCCCEGNMCNEKFSYFPEMEVTQPTS,
(SEQ ID NO: 181)
GRGEAETRECIYYNANWELERTNQSGLERCEGEQX₁KRRHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 182)
GRGEAETRECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 183)
GRGEAETRECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 184)
GRGEAETRECIYYNANWELERTNQSGLERCEGEQX₁KRLHCYASWRNSSGSIEIVKQGCWLDD
X₂X₃CYDRTDCVX₄X₅X₆X₇X₈PX₉VYFCCCEGNMCNEKFSYFPEMEVTQPTS,
(SEQ ID NO: 185)
GRGEAETRECIYYNANWELERTNQSGLERCEGEQX₁KRLHCYASWRNSSGTIEIVKQGCWLDD
X₂X₃CYDRTDCVX₄X₅X₆X₇X₈PX₉VYFCCCEGNMCNEKFSYFPEMEVTQPTS,
(SEQ ID NO: 186)
GRGEAETRECIYYNANWELERTNQSGLERCEGEQX₁KRLHCYASWRNSSGTIELVKKGCWLDD
X₂X₃CYDRTDCVX₄X₅X₆X₇X₈PX₉VYFCCCEGNMCNEKFSYFPEMEVTQPTS,
(SEQ ID NO: 187)
GRGEAETRECIYYNANWELERTNQSGLERCEGEQX₁KRLHCYASWRNSSGTIELVKKGCWLDD
X₂X₃CYDRQECVX₄X₅X₆X₇X₈PX₉VYFCCCEGNMCNEKFSYFPEMEVTQPTS,
(SEQ ID NO: 188)
GAILGRSETQECIYYNANWELERTNQSGLERCEGEQX₁KRRHCFATWKNISGSIEIVKQGCWLD
DX₂X₃CYDRTDCVX₄X₅X₆X₇X₈PX₉VYFCCCEGNMCNEKFSYFPEMEVTQPTS,
(SEQ ID NO: 189)
GAILGRSETQECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 190)
GAILGRSETQECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 191)
GAILGRSETQECIYYNANWELERTNQSGLERCEGEQX₁KRLHCYASWRNSSGSIEIVKQGCWLD
DX₂X₃CYDRTDCVX₄X₅X₆X₇X₈PX₉VYFCCCEGNMCNEKFSYFPEMEVTQPTS,
(SEQ ID NO: 192)
GAILGRSETQECIYYNANWELERTNQSGLERCEGEQX₁KRLHCYASWRNSSGTIEIVKQGCWLD
DX₂X₃CYDRTDCVX₄X₅X₆X₇X₈PX₉VYFCCCEGNMCNEKFSYFPEMEVTQPTS,
(SEQ ID NO: 193)
GAILGRSETQECIYYNANWELERTNQSGLERCEGEQX₁KRLHCYASWRNSSGTIELVKKGCWLD
DX₂X₃CYDRTDCVX₄X₅X₆X₇X₈PX₉VYFCCCEGNMCNEKFSYFPEMEVTQPTS,
and
(SEQ ID NO: 194)
GAILGRSETQECIYYNANWELERTNQSGLERCEGEQX₁KRLHCYASWRNSSGTIELVKKGCWLD
DX₂X₃CYDRQECVX₄X₅X₆X₇X₈PX₉VYFCCCEGNMCNEKFSYFPEMEVTQPTS,
wherein X₁ is D or R, X₂ is I, F, E, D, Y, S, N, Q, or T, X₃ is N or T, X₄ is A or E, X₅ is T or K, X₆ is E or K, X₇ is E or D, X₈ is N or S, and X₉ is Q, E, K, R, D, or N, optionally wherein the chimera is truncated from the N-terminus by deletion of 1, 2, 3, 4, 5, 6, 7, 8, or 9 amino acids, wherein the chimera retains the two amino acids before the first cysteine.

10-12. (canceled)

13. A method of slowing or inhibiting progression of a calcification disease, cardiomyopathy, right-sided heart failure, left-sided heart failure, backward heart failure, forward heart failure, high-output heart failure, low-output heart failure, compensated heart failure, decompensated heart failure, a heart valve disease, an arrhythmia, ventricular dysfunction, hyperlipidemia, hyperlipoproteinemia, vasculitis, deep vein thrombosis, sitosterolemia, cerebrotendinous xanthomatosis, familial hypobetalipoproteinemia, aortic aneurysm, pseudoaneurysm, intramural hematoma, xanthoma, xanthelasma, hepatosteatosis, mixed dyslipidemia, or hypertriglyceridemia in a subject in need thereof or preventing or reducing aneurysm formation in a subject in need thereof, the method comprising administering to the subject a therapeutically effective amount of:
a) a polypeptide comprising an extracellular ActRIIB variant, the variant having one or more amino acid substitutions relative to the sequence of GRGEAETRECIYYNANWELERTNQSGLERCEGEQDKRLHCYASWRNSSGTIELVKKGCWLDDFNCYDRQE CVATEENPQVYFCCCEGNFCNERFTHLPEAGGPEVTYEPPPTAPT (SEQ ID NO: 74), wherein the variant comprises one or more amino acid substitutions that impart reduced BMP9 binding relative to wild type extracellular ActRIIB and one or more additional amino acid substitutions, wherein the substitutions that reduce BMP9 binding comprise one or more of:
i) amino acid substitution E75K;
ii) amino acid substitutions Q69T and E70D; or
iii) amino acid substitutions Q69D and E70T,
optionally wherein the variant is truncated from the N-terminus by deletion of 1, 2, 3, 4, 5, 6, or 7 amino acids; or
b) a polypeptide comprising an extracellular ActRII chimera, the chimera having a sequence of any one of
(SEQ ID NO: 174)
GAILGRAETRECIYYNANWELERTNQSGLERCEGEQX₁KRRHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 175)
GAILGRAETRECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 176)
GAILGRAETRECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 177)
GAILGRAETRECIYYNANWELERTNQSGLERCEGEQX₁KRLHCYASWRNSSGSIEIVKQGCWLD
DX₂X₃CYDRTDCVX₄X₅X₆X₇X₈PX₉VYFCCCEGNMCNEKFSYFPEMEVTQPTS,
(SEQ ID NO: 178)
GAILGRAETRECIYYNANWELERTNQSGLERCEGEQX₁KRLHCYASWRNSSGTIEIVKQGCWLD
DX₂X₃CYDRTDCVX₄X₅X₆X₇X₈PX₉VYFCCCEGNMCNEKFSYFPEMEVTQPTS,
(SEQ ID NO: 179)
GAILGRAETRECIYYNANWELERTNQSGLERCEGEQX₁KRLHCYASWRNSSGTIELVKKGCWLD
DX₂X₃CYDRTDCVX₄X₅X₆X₇X₈PX₉VYFCCCEGNMCNEKFSYFPEMEVTQPTS,
(SEQ ID NO: 180)
GAILGRAETRECIYYNANWELERTNQSGLERCEGEQX₁KRLHCYASWRNSSGTIELVKKGCWLD
DX₂X₃CYDRQECVX₄X₅X₆X₇X₈PX₉VYFCCCEGNMCNEKFSYFPEMEVTQPTS,
(SEQ ID NO: 181)
GRGEAETRECIYYNANWELERTNQSGLERCEGEQX₁KRRHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 182)
GRGEAETRECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 183)
GRGEAETRECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 184)
GRGEAETRECIYYNANWELERTNQSGLERCEGEQX₁KRLHCYASWRNSSGSIEIVKQGCWLDD
X₂X₃CYDRTDCVX₄X₅X₆X₇X₈PX₉VYFCCCEGNMCNEKFSYFPEMEVTQPTS,
(SEQ ID NO: 185)
GRGEAETRECIYYNANWELERTNQSGLERCEGEQX₁KRLHCYASWRNSSGTIEIVKQGCWLDD
X₂X₃CYDRTDCVX₄X₅X₆X₇X₈PX₉VYFCCCEGNMCNEKFSYFPEMEVTQPTS,
(SEQ ID NO: 186)
GRGEAETRECIYYNANWELERTNQSGLERCEGEQX₁KRLHCYASWRNSSGTIELVKKGCWLDD
X₂X₃CYDRTDCVX₄X₅X₆X₇X₈PX₉VYFCCCEGNMCNEKFSYFPEMEVTQPTS,
(SEQ ID NO: 187)
GRGEAETRECIYYNANWELERTNQSGLERCEGEQX₁KRLHCYASWRNSSGTIELVKKGCWLDD
X₂X₃CYDRQECVX₄X₅X₆X₇X₈PX₉VYFCCCEGNMCNEKFSYFPEMEVTQPTS,
(SEQ ID NO: 188)
GAILGRSETQECIYYNANWELERTNQSGLERCEGEQX₁KRRHCFATWKNISGSIEIVKQGCWLD
DX₂X₃CYDRTDCVX₄X₅X₆X₇X₈PX₉VYFCCCEGNMCNEKFSYFPEMEVTQPTS,
(SEQ ID NO: 189)
GAILGRSETQECIYYNANWELERTNQSGLERCEGEQX₁KRLHCFATWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 190)
GAILGRSETQECIYYNANWELERTNQSGLERCEGEQX₁KRLHCYASWKNISGSIEIVKQGCWLDD
X₂X₃CYDRTDCVX₄X₅X₆X₇X₈PX₉VYFCCCEGNMCNEKFSYFPEMEVTQPTS,
(SEQ ID NO: 191)
GAILGRSETQECIYYNANWELERTNQSGLERCEGEQX₁KRLHCYASWRNSSGSIEIVKQGCWLD
DX₂X₃CYDRTDCVX₄X₅X₆X₇X₈PX₉VYFCCCEGNMCNEKFSYFPEMEVTQPTS,
(SEQ ID NO: 192)
GAILGRSETQECIYYNANWELERTNQSGLERCEGEQX₁KRLHCYASWRNSSGTIEIVKQGCWLD
DX₂X₃CYDRTDCVX₄X₅X₆X₇X₈PX₉VYFCCCEGNMCNEKFSYFPEMEVTQPTS,
(SEQ ID NO: 193)
GAILGRSETQECIYYNANWELERTNQSGLERCEGEQX₁KRLHCYASWRNSSGTIELVKKGCWLD
DX₂X₃CYDRTDCVX₄X₅X₆X₇X₈PX₉VYFCCCEGNMCNEKFSYFPEMEVTQPTS,
and
(SEQ ID NO: 194)
GAILGRSETQECIYYNANWELERTNQSGLERCEGEQX₁KRLHCYASWRNSSGTIELVKKGCWLD
DX₂X₃CYDRQECVX₄X₅X₆X₇X₈PX₉VYFCCCEGNMCNEKFSYFPEMEVTQPTS,
wherein X₁ is D or R, X₂ is I, F, E, D, Y, S, N, Q, or T, X₃ is N or T, X₄ is A or E, X₅ is T or K, X₆ is E or K, X₇ is E or D, X₈ is N or S, and X₉ is Q, E, K, R, D, or N, optionally wherein the chimera is truncated from the N-terminus by deletion of 1, 2, 3, 4, 5, 6, 7, 8, or 9 amino acids, wherein the chimera retains the two amino acids before the first cysteine.

14. (canceled)

15. The method of claim 1, wherein:
a) the calcification disease is Monckeberg's vascular calcification disease, vascular calcification, or valvular calcification;
b) the cardiomyopathy is dilated cardiomyopathy, hypertrophic cardiomyopathy, restrictive cardiomyopathy, arrhythmogenic right ventricular dysplasia, unclassified cardiomyopathy, pediatric cardiomyopathy, or peripartum cardiomyopathy;
c) the left-sided heart failure is heart failure with reduced ejection fraction (HFrEF) or heart failure with preserved ejection fraction (HFpEF);
d) the heart failure is chronic;
e) the heart failure is acute;
f) the heart valve disease is aortic stenosis, mitral valve insufficiency, mitral valve prolapse, or rheumatic heart disease;
g) the arrythmia is atrial flutter or atrial fibrillation, a ventricular arrythmia, cardiac arrhythmia associated with myocardial ischemia or myocardial infarction, or arrhythmia associated with cerebral hemorrhage;
h) the hyperlipidemia or hyperlipoproteinemia is congenital hyperlipidemia or hyperlipoproteinemia;
i) the hyperlipidemia or hyperlipoproteinemia is acquired hyperlipidemia or hyperlipoproteinemia; or
j) the ventricular dysfunction is left ventricular dysfunction.

16-19. (canceled)

20. The method of claim 1, wherein the subject:
a has New York Heart Association (NYHA) Functional Class II to IV symptoms;
b) has elevated N-terminal pro b-type natriuretic peptide (NT-proBNP); or
c) is on a Heart Failure (HF) background therapy.

21-23. (canceled)

24. The method of claim 15, wherein the congenital hyperlipidemia or hyperlipoproteinemia is familial combined hyperlipidemia (FCHL), primary hyperlipoproteinemia, hyperapobetalipoproteinemia, Frederickson type III hyperlipidemia (familial dysbetalipoprotenemia), Frederickson type IV hyperlipidemia (familial hypertriglyceridemia), Frederickson type V hyperlipidemia (endogenous hypertriglyceridemia), or small dense LDL syndrome (LDL phenotype B).

25-27. (canceled)

28. The method of claim 1, wherein the method further comprises administering a HF background therapy.

29. (canceled)

30. The method of claim 1, wherein the method:
a) reduces fibrosis, reduces inflammation, reduces cardiac remodeling, improves cardiac function, or improves ventricular function;
b) slows or inhibits progression of the disease or condition;
c) delays development of or prevents the disease or condition;
d) reduces, delays, or eliminates the need for coronary revascularization, heart valve repair or replacement, implantation of a cardioverter-defibrillator, cardiac resynchronization therapy, a ventricular assist device, or a heart transplant;
e) reduces the risk of cardiovascular death;
f) reverses progression of the disease or condition;
g) decreases NT-proBNP levels;
h) improves cardiopulmonary exercise testing (CPET) parameters; or
i) improves echocardiographic (ECHO) parameters.

31-41. (canceled)

42. The method of claim 9, wherein:
a) the calcification disease is Monckeberg's vascular calcification disease, vascular calcification, or valvular calcification;
b) the cardiomyopathy is dilated cardiomyopathy, hypertrophic cardiomyopathy, restrictive cardiomyopathy, arrhythmogenic right ventricular dysplasia, unclassified cardiomyopathy, pediatric cardiomyopathy, or peripartum cardiomyopathy;
c) the left-sided heart failure is HFrEF or HFpEF;
d) the heart failure is chronic;
e) the heart failure is acute;
f) the heart valve disease is aortic stenosis, mitral valve insufficiency, mitral valve prolapse, or rheumatic heart disease;
g) the arrythmia is atrial flutter or atrial fibrillation, a ventricular arrythmia, cardiac arrhythmia associated with myocardial ischemia or myocardial infarction, or arrhythmia associated with cerebral hemorrhage;
h) the hyperlipidemia or hyperlipoproteinemia is congenital hyperlipidemia or hyperlipoproteinemia;
i) the hyperlipidemia or hyperlipoproteinemia is acquired hyperlipidemia or hyperlipoproteinemia; or
j) the ventricular dysfunction is left ventricular dysfunction.

43. The method of claim 42, wherein the congenital hyperlipidemia or hyperlipoproteinemia is FCHL, primary hyperlipoproteinemia, hyperapobetalipoproteinemia, Frederickson type III hyperlipidemia (familial dysbetalipoprotenemia), Frederickson type IV hyperlipidemia (familial hypertriglyceridemia), Frederickson type V hyperlipidemia (endogenous hypertriglyceridemia), or small dense LDL syndrome (LDL phenotype B).

44. The method of claim 9, wherein the subject:
a) has NYHA Functional Class II to IV symptoms;
b) has elevated NT-proBNP; or
c) is on a HF background therapy.

45. The method of claim 9, wherein the method further comprises administering a HF background therapy.

46. The method of claim 9, wherein the method:
a) reduces fibrosis, reduces inflammation, reduces cardiac remodeling, improves cardiac function, or improves ventricular function;
b) slows or inhibits progression of the disease or condition;
c) delays development of or prevents the disease or condition;
d) reduces, delays, or eliminates the need for coronary revascularization, heart valve repair or replacement, implantation of a cardioverter-defibrillator, cardiac resynchronization therapy, a ventricular assist device, or a heart transplant;
e) reduces the risk of cardiovascular death;
f) reverses progression of the disease or condition;
g) decreases NT-proBNP levels;
h) improves CPET parameters; or
i) improves ECHO parameters.

47. The method of claim 13, wherein:
a) the calcification disease is Monckeberg's vascular calcification disease, vascular calcification, or valvular calcification;
b) the cardiomyopathy is dilated cardiomyopathy, hypertrophic cardiomyopathy, restrictive cardiomyopathy, arrhythmogenic right ventricular dysplasia, unclassified cardiomyopathy, pediatric cardiomyopathy, or peripartum cardiomyopathy;
c) the left-sided heart failure is HFrEF or HFpEF;
d) the heart failure is chronic;
e) the heart failure is acute;
f) the heart valve disease is aortic stenosis, mitral valve insufficiency, mitral valve prolapse, or rheumatic heart disease;
g) the arrythmia is atrial flutter or atrial fibrillation, a ventricular arrythmia, cardiac arrhythmia associated with myocardial ischemia or myocardial infarction, or arrhythmia associated with cerebral hemorrhage;
h) the hyperlipidemia or hyperlipoproteinemia is congenital hyperlipidemia or hyperlipoproteinemia;
i) the hyperlipidemia or hyperlipoproteinemia is acquired hyperlipidemia or hyperlipoproteinemia; or
j) the ventricular dysfunction is left ventricular dysfunction.

48. The method of claim 47, wherein the congenital hyperlipidemia or hyperlipoproteinemia is FCHL, primary hyperlipoproteinemia, hyperapobetalipoproteinemia, Frederickson type III hyperlipidemia (familial dysbetalipoprotenemia), Frederickson type IV hyperlipidemia (familial hypertriglyceridemia), Frederickson type V hyperlipidemia (endogenous hypertriglyceridemia), or small dense LDL syndrome (LDL phenotype B).

49. The method of claim 13, wherein the subject:
a) has NYHA Functional Class II to IV symptoms;
b) has elevated NT-proBNP; or
c) is on a HF background therapy.

50. The method of claim 13, wherein the method further comprises administering a HF background therapy.

51. The method of claim 13, wherein the method:
a) reduces fibrosis, reduces inflammation, reduces cardiac remodeling, improves cardiac function, or improves ventricular function;
b) slows or inhibits progression of the disease or condition;
c) delays development of or prevents the disease or condition;
d) reduces, delays, or eliminates the need for coronary revascularization, heart valve repair or replacement, implantation of a cardioverter-defibrillator, cardiac resynchronization therapy, a ventricular assist device, or a heart transplant;
e) reduces the risk of cardiovascular death;
f) reverses progression of the disease or condition;
g) decreases NT-proBNP levels;
h) improves CPET parameters; or
i) improves ECHO parameters.
